(19) United States
(12) Patent Application Publication
(10) Pub. No.: US 2022/0298507 A1
(43) Pub. Date: Sep. 22, 2022

(54) COMPOSITIONS AND METHODS FOR RNA INTERFERENCE

(71) Applicants: Chan Zuckerberg Biohub, Inc., San Francisco, CA (US); The Board of Trustees of the Leland Stanford Junior University, Stanford, CA (US); University of Massachusetts, Boston, MA (US)

(72) Inventors: William J. Greenleaf, Stanford, CA (US); Phillip D. Zamore, Stanford (US); Winston R. Becker, Stanford, CA (US); Benjamin Ober-Reynolds, Stanford, CA (US); Karina Jouravleva, Boston, MA (US); Samson M. Jolly, Boston, MA (US)

(21) Appl. No.: 17/616,750

(22) PCT Filed: Jun. 10, 2020
(86) PCT No.: PCT/US2020/036919

§ 371 Date: Dec. 6, 2021

Related U.S. Application Data
(60) Provisional application No. 62/859,995, filed on Jun. 11, 2019.

(51) Int. Cl. C12N 15/113

(52) U.S. Cl. CPC C12N 15/113; C12N 2310/113; C12N 2310/141; C12N 2310/344; C12N 2310/533; C12N 2320/10; C12N 2330/31

(57) ABSTRACT

The disclosure provides inhibitory RNA polynucleotides that have partial complementarity to a target gene. The inhibitory RNA polynucleotides have at least one mismatched nucleotide and can be designed to increase or decrease the cleavage rate when loaded onto the RNA-induced silencing complex (RISC).

### CROSS-REFERENCES TO RELATED APPLICATIONS

This application claims priority to U.S. Provisional Application No. 62/859,995, filed Jun. 11, 2019, the disclosure of which is hereby incorporated by reference in its entirety for all purposes.

### STATEMENT AS TO RIGHTS TO INVENTIONS MADE UNDER FEDERALLY SPONSORED RESEARCH AND DEVELOPMENT

This invention was made with government support under grant no. GM062862 awarded by the National Institutes of Health. The Government has certain rights in the invention.

### BACKGROUND

Therapeutics using RNA interference hold great potential in treating various diseases, especially diseases caused by a mutated or aberrant gene. Methods of designing RNA interference molecules are useful to generate effective therapeutics.

### SUMMARY

In one aspect, the disclosure features an inhibitory RNA polynucleotide comprising between 15 and 30 nucleotides, wherein the inhibitory RNA polynucleotide is partially complementary to an equal length portion of a target gene, wherein the inhibitory RNA polynucleotide comprises at least one mismatched nucleotide at position 5, 7, 8, 12, 16, 17, 18, 19, 20, or 21, and wherein the inhibitory RNA polynucleotide guides an RNA-induced silencing complex (RISC) to cleave the target gene.

In some embodiments, the inhibitory RNA polynucleotide comprises at least one mismatched nucleotide at position 12, 16, 17, 18, 19, 20, or 21. In certain embodiments, the inhibitory RNA polynucleotide comprises one mismatched nucleotide at position 12. In certain embodiments, the inhibitory RNA polynucleotide comprises one mismatched nucleotide at position 18. In further embodiments, the inhibitory RNA polynucleotide comprises at least two mismatched nucleotides at positions selected from positions 5, 7, 8, 12, 15, 16, 17, 18, 19, 20, and 21.

In some embodiments, the inhibitory RNA polynucleotide comprises two mismatched nucleotides, in which a first mismatched nucleotide is at position 12 and a second mismatched nucleotide is at position 5, 7, 8, 15, 16, 17, 18, 19, 20, or 21. In some embodiments, the inhibitory RNA polynucleotide comprises two mismatched nucleotides, in which a first mismatched nucleotide is at position 18 and a second mismatched nucleotide is at position 5, 7, 8, 12, 15, 16, 17, 19, 20, or 21. In particular embodiments, the inhibitory RNA polynucleotide comprises two mismatched nucleotides, in which a first mismatched nucleotide is at position 12 and a second mismatched nucleotide is at position 18.

In some embodiments, the inhibitory RNA polynucleotide comprises at least two mismatched nucleotides at positions selected from positions 15, 16, 17, 18, 19, 20, and 21. In further embodiments, the inhibitory RNA polynucleotide comprises mismatched nucleotides at positions 15, 16, 17, 18, 19, 20, and 21 (e.g., positions 17, 18, 19, 20, and 21).

In some embodiments of this aspect of the disclosure, the inhibitory RNA polynucleotide guides the RISC to cleave the target gene at a faster cleavage rate than the corresponding cleavage rate of RISC when RISC is guided by a corresponding inhibitory RNA polynucleotide having complete complementarity to the equal length portion of the target gene.

In some embodiments, the inhibitory RNA polynucleotide is single-stranded. In other embodiments, the inhibitory RNA polynucleotide is double-stranded.

In another aspect, the disclosure features a pharmaceutical composition comprising an inhibitory RNA polynucleotide described herein and a pharmaceutically acceptable carrier. In some embodiments, the inhibitory RNA polynucleotide is encapsulated in a nanoparticle, such as a liposome. In certain embodiments, the liposome is a polyethylene glycol (PEG) liposome.

In another aspect, the disclosure features a method of increasing the cleavage rate of an RNA-induced silencing complex (RISC) in cleaving a target gene, comprising introducing at least one mismatched nucleotide to an inhibitory RNA polynucleotide comprising between 15 and 30 nucleotides, wherein the inhibitory RNA polynucleotide is partially complementary to an equal length portion of a target gene, wherein the inhibitory RNA polynucleotide comprises at least one mismatched nucleotide at position 5, 7, 8, 12, 16, 17, 18, 19, 20, or 21, and wherein the RISC is guided by the inhibitory RNA polynucleotide to bind and cleave the target gene at a faster cleavage rate than the corresponding cleavage rate of RISC when RISC is guided by a corresponding inhibitory RNA polynucleotide having complete complementarity to the equal length portion of the target gene.

In some embodiments of the method, the inhibitory RNA polynucleotide comprises at least two mismatched nucleotides at positions selected from positions 5, 7, 8, 12, 15, 16, 17, 18, 19, 20, and 21.

In another aspect, the disclosure features a method of decreasing the cleavage rate of an RNA-induced silencing complex (RISC) in cleaving a target gene, comprising introducing at least two mismatched nucleotides to an inhibitory RNA polynucleotide comprising between 15 and 30 nucleotides, wherein the inhibitory RNA polynucleotide is partially complementary to an equal length portion of a target gene, wherein the inhibitory RNA polynucleotide comprises at least two mismatched nucleotides at positions selected from positions 9, 10, 11, and 13, and wherein the RISC is guided by the inhibitory RNA polynucleotide to bind and cleave the target gene at a slower cleavage rate than the corresponding cleavage rate of RISC when RISC is guided by a corresponding inhibitory RNA polynucleotide having complete complementarity to the equal length portion of the target gene.

In yet another aspect, the disclosure features a method of decreasing the expression level of a target gene in a cell, comprising contacting the cell with an inhibitory RNA polynucleotide comprising between 15 and 30 nucleotides, wherein the inhibitory RNA polynucleotide is partially complementary to an equal length portion of a target gene, wherein the inhibitory RNA polynucleotide comprises at least one mismatched nucleotide at position 5, 7, 8, 12, 16, 17, 18, 19, 20, or 21, and wherein the inhibitory RNA polynucleotide guides an RNA-induced silencing complex (RISC) to cleave the target gene.

In a further aspect, the disclosure features a method of treating a disease in a subject in need thereof, comprising administered to the subject an inhibitory RNA polynucleotide, wherein the inhibitory RNA polynucleotide is partially complementary to an equal length portion of a target gene associated with the disease and comprises at least one mismatched nucleotide at position 5, 7, 8, 12, 16, 17, 18, 19, 20, or 21, and wherein the inhibitory RNA polynucleotide decreases the expression level of the target gene. In some embodiments, the inhibitory RNA polynucleotide comprises at least two mismatched nucleotides at positions selected from positions 5, 7, 8, 9, 10, 12, 13, 15, 16, 17, 18, 19, 20, and 21.

In a further aspect, the disclosure features a method of synthesizing an inhibitory RNA polynucleotide with an increased cleavage rate for a target gene, comprising: (a) providing a sequence of the target gene; (b) selecting a portion of the sequence of the target gene where the inhibitory RNA polynucleotide binds; (c) selecting at least one position from positions 5, 7, 8, 12, 16, 17, 18, 19, 20, and 21 of the inhibitory RNA polynucleotide to introduce a mismatched nucleotide at the position; and (c) introducing the mismatched nucleotide at the selected position of the inhibitory RNA polynucleotide during synthesis of the inhibitory RNA polynucleotide, wherein the inhibitory RNA polynucleotide is partially complementary to an equal length portion of the target gene, and wherein an RNA-induced silencing complex (RISC) is guided by the inhibitory RNA polynucleotide to bind and cleave the target gene at a faster cleavage rate than the corresponding cleavage rate of RISC when RISC is guided by a corresponding inhibitory RNA polynucleotide having complete complementarity to the equal length portion of the target gene.

### BRIEF DESCRIPTION OF THE DRAWINGS

FIGS. 1A-1E: High-throughput Characterization of RISC Binding to in situ Transcribed RNA. FIGS. 1A and 1B: Schematic of RISC binding to in situ transcribed RNA targets tethered to DNA clusters within a sequenced flow cell and associated representative experimental images. FIG. 1A shows hybridization of a fluorescent DNA oligonucleotide to tethered RNA molecules. FIG. 1B shows fluorescently labeled RISC binding to RNA targets at different time points for two RISC concentrations. FIG. 1C: Summary of targets profiled within the let-7a target library. Total number of targets of each class are indicated by the sum of the light blue, dark blue, and gray bars. The number of targets with an affinity <10 pM are shown in dark blue, the number of targets with affinities ranging between 10 pM and 4 nM are shown in light blue, and targets with affinity greater than 4 nM are shown in gray. The number of variants for which association was measured for at least three concentrations is shown in orange. FIG. 1D: A representative set of fit curves for multiple RISC association experiments for a single target sequence. Y-axis indicates the normalized fluorescence. Error bars correspond to the 95% confidence interval on the median fluorescence at each time point. The smaller plot to the right shows the linear relationship between RISC concentration and observed binding rate, from which the association rate was determined. The colors of the curves and points in the plots represents the concentrations that the association data was collected at, with lighter blue indicating a higher concentration. In the schematic the guide is in gray and the target in blue. FIG. 1E: Representative binding isotherms fit to the normalized fluorescence values. Schematic to the right shows the RISC targets plotted in corresponding colors, which have different degrees of complementarity to the guide (in gray).

FIGS. 2A and 2B: AGO2 Library Design and Construction. FIG. 2A: Schematic of designed targets included in Ago libraries. FIG. 2B: Construct used in array experiments. Transcribed region is indicated above the schematic.

FIGS. 3A-3G: Sequence Determinants of AGO2 Association Kinetics. FIG. 3A: Association rates for miR 21 (upper left) and let-7a (lower right) loaded RISC binding to single and double mismatched targets. Axes are labeled with the 3′ end of the target (5′ end of the guide) starting at 1. Colors are centered on the control association rate (gray) with blue representing faster and red representing slower. Gray crosses represent missing data. FIG. 3B: Association rate for tandem double mismatches mapped onto the AGO2 crystal structure (PDB ID: 4W5N). FIG. 3C: Association rates for miR 21 (upper left) and let-7a (lower right) targets containing different length stretches of mismatches in which the target nucleotides were substituted with their complementary nucleotide. Examples are shown for mismatch stretches 2-4 and 5-9 at the top of the panel. For the 2-4 mismatches, the corresponding targets in the heat map are located at the intersection of 2 on the ‘beginning complement mismatch’ axis and 4 on the ‘ending complement mismatch’ axis. Colors are scaled as in panel A. FIG. 3D: Association rates for tandem triple mismatches of miR 21 targets. Each boxplot includes the 27 triple substitutions for the three target bases indicated on the x-axis. The dotted line represents the association rate to a fully complementary target. FIG. 3E: Effects of flanking structure on association rates. Schematics of the library constructs are shown at the right of the panel. The library elements include perfect complement miR 21 targets with increasingly long hairpins bound to either the seed (blue) or non-seed (orange) end of the target sequence. For each length of complementarity to the target sequence, there are up to five corresponding stem loops of different lengths prior to complementarity to the sequence. The plot shows the relationship between the number of complementary bases to one end of the target sequence and the resulting association rate. The dotted line represents the association rate to a fully complementary target. FIG. 3F: Association rates for miR 21 targets containing 1-3 insertions of each base. The dotted line represents the association rate to a fully complementary target. FIG. 3G: Association kinetics for miR 21 loaded RISC to targets containing single and double deletions. The dotted line represents the association rate to a fully complementary target.

FIGS. 4A-4E: Factors Contributing to AGO2 Association Kinetics. FIG. 4A: Association rates for let-7 loaded RISC binding to targets with consecutive triple mismatches. FIG. 4B: Internal structure for double mismatched target sequences of miR 21 and let-7 as predicted by RNAfold. FIG. 4C: Predicted secondary structures for miR 21 target containing t1G and t12G substitutions. The ensemble free energy predicted by RNAfold is shown below each structure. FIG. 4D: Association kinetics for let-7 loaded RISC to targets containing single and double target deletions. FIG. 4E: Association rates for let-7 targets containing 1-5 insertions of each base.

FIGS. 5A-5E: Target Sequence Contributions to AGO2 Binding Energies. FIG. 5A: Binding energies for miR 21 (upper left) and let-7a (lower right) loaded RISC binding to single and double mismatched targets. Axes are labeled with the 3′ end of the target (5′ end of the guide) starting at 1. White boxes represent missing data. FIG. 5B: Binding energies for miR 21 (upper left) and let-7a (lower right) targets containing different length stretches of mismatches. All mismatches shown were generated by substituting the target bases with their complementary bases (e.g., A to U). FIG. 5C: Binding affinities for targets containing progressively more complementarity to RISC. FIG. 5D: Effect of tandem triple substitutions in the target sequence on miR 21 (top) and let-7a (bottom) binding affinity. Dashed lines indicate the limits of detection and the numbers above and below the line indicate the number of targets in each group that fell beyond those limits. FIG. 5E: Binding affinities for RISC loaded with miR 21 (top) or let-7a (bottom) to targets with 1-7 nucleotides insertions. Dashed lines indicate the limits of detection and points below the line all bound with higher affinity than the detection limit.

FIGS. 6A-6K: Binding Affinity of AGO2 to Predicted and Designed Targets. FIGS. 6A and 6B: RISC binding affinity to miR 21 (FIG. 6A) and let 7a (FIG. 6B) targets containing two by two substitutions. A schematic of the targets is shown at the top of the panel. Transition (A↔G and C↔U) substitutions are above the diagonal and complement (C↔G and A↔U) substitutions are shown below. FIGS. 6C and 6D: RISC binding affinity to miR 21 (FIG. 6C) and let 7a (FIG. 6D) targets containing three by three substitutions. A schematic of the targets is shown at the top of the panel. FIGS. 6E and 6F: Affinities measured for let-7a (FIG. 6E) and miR 21 (FIG. 6F) loaded RISC binding to all predicted targets grouped by the site type. Dashed lines represent the minimum binding affinity that we could resolve experimentally. FIGS. 6G and 6H: Affinities measured for let 7a (FIG. 6G) and miR 21 (FIG. 6H) predicted targets filtered to keep those that are predicted to form less internal structure. FIG. 6I: Relationship between measured target affinity and RNAfold predicted internal structure. FIG. 6J: Relationship between binding affinity and mean (±SEM) change in target abundance following transfection of a let-7a decoy for canonical seed types. 12fc: log2 fold change. FIG. 6K: Highest affinity noncanonical targets measured for let-7a RISC, related to FIGS. 6E and 6G.

FIGS. 7A-7D: AGO2 Cleave 'n-Seq (CNS) Enables High-throughput Measurement of Single Turnover Cleavage Kinetics. FIG. 7A: Method to determine single turnover cleavage rates for AGO2 targets. A dsDNA library was transcribed into RNA that was subsequently incubated with a 10-fold excess of AGO2 for a range of times. The reactions were quenched at −80° C. and the protein was denatured at 95° C. The resulting pools of uncut RNA were reverse transcribed and barcoded for sequencing. FIG. 7B: Cleavage rates for miR 21 (upper left) and let-7a (lower right) targets with single and double substitutions. Deep red represents targets for which no detectable cleavage was observed. Targets colored in blue were cleaved faster than the fully complementary target. FIG. 7C: Cleavage rates of miR 21 (upper left) and let-7a (lower right) targets containing different length stretches of mismatches. All mismatched shown are generated by substituting the target bases with their complementary bases (e.g., A to U). FIG. 7D: Cleavage rates for miR 21 (top) and let 7a (bottom) targets containing three consecutive substitutions. The black dotted line represents the cleavage rate of the fully complementary RNA target, whereas the gray dotted line indicates the cleavage rate detection limit. The numbers at the bottom of the plot represent the number of targets in each group for which no detectable cleavage was observed.

FIGS. 8A-8I: AGO2 Cleavage Kinetics. FIG. 8A: Fraction of uncleaved RNA for 3 target sequences as a function of time obtained from the RISC-Cleave 'n Seq experiments. The lines represent the fit to a single exponential. FIG. 8B: Simulations of cleavage rates for different association rates. FIG. 8C: Simulated relationship between fit cleavage rate and true cleavage rate for different association rates. FIG. 8D: Cleavage rates measured for perfectly complementary targets with different 5 nucleotide flanking sequences. FIG. 8E: Cleavage rates of target sequences containing double deletions for miR 21 (top left) and let 7a (lower right). FIGS. 8F and 8G: Cleavage rates of miR 21 (FIG. 8F) and let 7a (FIG. 8G) targets containing single and consecutive deletions. FIGS. 8H and 8I: Cleavage rates of miR 21 (FIG. 8H) and let 7a (FIG. 8I) targets containing multiple nucleotides inserted into the target sequence.

FIGS. 9A-9D: Insertions and Deletions Have Similar Effects on RISC Binding. FIG. 9A: Association rates for miR 21 single insertions (blue dots) and single deletions (white dots). Insertions and deletions that correspond to multiple target positions, such as insertion of the same base either before or after a base, or deletion of a repeated base, are plotted in all possible target positions. The fully complementary sequence is shown on top of each plot, and its association rate is indicated by the dotted line. Gray line, all single deletions; blue line, mean of the single insertions. FIG. 9B: Same as (FIG. 9A), but for let 7a. FIG. 9C: Binding affinity for miR 21 single insertions and single deletions. Points below the solid line were too high affinity to be accurately measured (K_{D}<10 pM). FIG. 9D: Same as (FIG. 9C), but for let 7a.

FIGS. 10A-10C: Target Insertions and Deletions Result in Out of Phase Trends for Cleavage Rates. FIG. 10A: Cleavage rates for miR 21 single insertions (blue dots) and single deletions (white dots). Insertions and deletions that correspond to multiple target positions, such as insertion of the same base either before or after a base, or deletion of a repeated base, are plotted in all possible target positions. The perfectly complementary sequence is shown on top of each plot, and the cleavage rate of the fully complementary target is indicated by the dotted line. Targets for which no cleavage was detected are plotted below the solid black line. The gray line traces all single deletions, while the blue line traces the mean of the single insertions. FIG. 10B: Same as (FIG. 10A), but for let-7a. FIG. 10C: let-7a cleavage rates were mapped onto the RNA components of the AGO2 crystal structure (PDB ID: 4W5O). Target insertions were mapped onto the 9mer RNA target such that the mean of all insertions between t1 and t2 are mapped onto t1. Single deletion cleavage rates were mapped onto the guide strand of the structure. Cleavage rates near the wild-type rate are colored white, while immeasurably slow cleavage rates are colored deep red. The first frame shows both the guide and target strands as they enter the central cleft of the protein, while the second frame shows only the guide strand. The third frame shows the guide strand as it exits the central cleft of the protein.

FIGS. 11A-11H: Predictive Models for AGO2 Binding Affinity and Cleavage Kinetics. FIG. 11A: Schematic of alignment of guide and target sequences to identify bound orientation. FIGS. 11B and 11C: Comparison of binding affinity predicted by let 7a (FIG. 11B) and miR 21 (FIG. 11C) specific models to observed binding affinities. FIGS. 11D and 11E: Comparison of cleavage rates predicted by let 7a (FIG. 11D) and miR 21 (FIG. 11E) specific models to observed cleavage rates. The color of the points represents the density of points at that position, with yellow being the densest and purple being the least dense. FIG. 11F: Comparison of cleavage rates predicted by a general cleavage model to observed cleavage rates. FIG. 11G: Parameters obtained from fitting miR 21 cleavage model. FIG. 11H: Parameters obtained by fitting a general cleavage model.

FIGS. 12A-12G: Models of AGO2 Binding Affinity and Cleavage Kinetics. FIG. 12A: Overview of dynamic programming alignment algorithm used to align sequences. FIG. 12B: Predicted double substitution cleavage rates from single substitution cleavage rates. FIGS. 12C-12E: Performance of cleavage model when trained and tested on a random split of the data for let 7a cleavage model (FIG. 12C), miR 21 cleavage model (FIG. 12D), and general cleavage model (FIG. 12E). More complicated models performed only marginally better (FIGS. 12F and 12G).

FIGS. 13A-14H: Additional in Cell Target Knockdown Analysis. FIG. 13A: CRISPR Cas9 editing design to generate the miR 21 knockout cell line. Guide RNAs were designed to cut both sides of the primary miR 21 hairpin. Successful editing was confirmed by PCR of the edited region and confirming loss of the 72nt hairpin sequence, as well as by a TaqMan assay specific for mature miR 21. FIG. 13B: Kinetic model of in cell RISC activity. C is the dissociation rate scaling factor. FIGS. 13C-13E: Additional demonstrations of kinetic biochemical model of miR 21 knockdown. The miR 21 transfection concentration is indicated above each panel. Individual targets are colored according to their measured cleavage rates. The dotted lines each have a slope of −1 and an intercept of 0. FIG. 13F: Knockdown for miR 21 targets containing 1-3 insertions of each base. FIG. 13G: Knockdown of all miR 21 double mismatched targets. Color bar is centered on the knockdown of a perfectly complementary target in the poly(A) sequence context. FIG. 13H: Knockdown of perfectly complementary targets in varying 5′ and 3′ sequence contexts. Color bar as in (FIG. 13G), with the poly(A) context perfectly complementary sequence as a reference.

FIGS. 14A-14F: Binding Affinity and Cleavage Rate Affect Knockdown in Cells. FIG. 14A: Scheme used to measure change in abundance of miR 21 targets in HEK-293 cells. After 48 h, RNA was isolated from cells, and uncleaved target RNA was sequenced. FIG. 14B: Comparison of normalized counts obtained from replicate miR 21 siRNA transfection experiments at the same concentration. FIG. 14C: Biochemical model for predicting siRNA knockdown from measured kₒₙ and k_{cleave}, and predicted k_{off} of each target. Sample shown is from the highest miR 21 transfection concentration (100 nM). Individual targets are colored by measured cleavage rate. Red dot, perfectly complementary target. Dotted line has slope=−1 and intercept=0. FIG. 14D: Change in abundance of targets bearing single mismatches at each miR 21 siRNA concentration transfected. FIG. 14E: siRNA-directed reduction in abundance of miR 21 targets with single insertions (blue dots) or deletions (white dots). Insertions and deletions that correspond to multiple target positions are plotted in all possible target positions. Dotted line, target fully complementary to the siRNA. Gray line, all single deletions; blue line, mean of the single insertions. FIG. 14F: siRNA-directed reduction in abundance for all tandem, doubly mismatched targets. Dotted line, target fully complementary to the siRNA.

### DETAILED DESCRIPTION OF THE EMBODIMENTS

### I. Introduction

Argonaute proteins loaded with microRNAs (miRNAs) or small interfering RNAs (siRNAs) form the RNA-Induced Silencing Complex (RISC), which represses target RNA expression. Predicting the biological targets, specificity, and efficiency of both miRNAs and siRNAs has been hamstrung by an incomplete understanding of the sequence determinants of RISC binding and cleavage. As described herein, high-throughput methods were applied to measure the association kinetics, equilibrium binding energies, and single-turnover cleavage rates of RISC. The experimental data uncover specific guide:target nucleotide mismatches that enhance the rate of target cleavage, suggesting unique siRNA design strategies. Using these data, quantitative models for RISC binding and target cleavage were derived. The in vitro measurements and models predict target gene knock-down in an engineered cellular system.

### II. Definitions

As used herein, the term “inhibitory RNA polynucleotide” refers to a small non-coding RNA molecule that functions in target gene silencing (e.g., RNA silencing).

As used herein, the term “mismatched nucleotide” refers to a nucleotide at a specific position in the inhibitory RNA polynucleotide that does not engage in Watson-Crick base pairing with a nucleotide at the corresponding position in the target gene when the inhibitory RNA polynucleotide hybridizes to an equal length portion of the target gene.

As used herein, the term “complementary” or “complementarity” refers to the capacity for base pairing via Watson-Crick hydrogen bonding interactions between nucleobases, nucleosides, or nucleotides of an inhibitory RNA polynucleotide to the nucleobases, nucleosides, or nucleotides at the corresponding positions of a target gene. In some embodiments, the inhibitory RNA polynucleotide can have complete complementarity to an equal length portion of the target gene, which means that all of the nucleotides in the inhibitory RNA polynucleotide are complementary to the nucleotides at the corresponding positions of the target gene. In some embodiments, the inhibitory RNA polynucleotide can have partial complementarity to an equal length portion of the target gene, which means that at least one of the nucleotides in the inhibitory RNA polynucleotide does not form Watson-Crick hydrogen bonding with the nucleotide at the corresponding position of the target gene.

As used herein, the term “pharmaceutical composition” refers to a medicinal or pharmaceutical formulation that contains an active ingredient as well as one or more excipients and diluents to enable the active ingredient suitable for the method of administration. The pharmaceutical composition of the present disclosure includes pharmaceutically acceptable components that are compatible with the inhibitory RNA polynucleotide. The pharmaceutical composition may be in aqueous form for intravenous or subcutaneous administration or in tablet or capsule form for oral administration.

As used herein, the term “pharmaceutically acceptable carrier” refers to an excipient or diluent in a pharmaceutical composition. The pharmaceutically acceptable carrier should be compatible with the other ingredients of the formulation and not deleterious to the recipient. In the present disclosure, the pharmaceutically acceptable carrier should provide adequate pharmaceutical stability to the inhibitory RNA polynucleotide. The nature of the carrier differs with the mode of administration. For example, for intravenous administration, an aqueous solution carrier is generally used; for oral administration, a solid carrier is preferred.

As used herein, the term “therapeutically effective amount” refers to an amount, e.g., pharmaceutical dose, effective in inducing a desired biological effect in a subject or patient or in treating a patient having a disease. It is also to be understood herein that a “therapeutically effective amount” may be interpreted as an amount giving a desired therapeutic effect, either taken in one dose or in any dosage or route, taken alone or in combination with other therapeutic agents. A therapeutically effective amount may be an amount that treats, prevents, alleviates, abates, or reduces the severity of symptoms of diseases and disorders.

As used herein, the term “treating” refers to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. Therapeutic benefit can also mean to effect a cure of one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. In particular embodiments, beneficial results that may be obtained from the methods for treating a disease that is caused, related to, or aggravated by a target gene by administering to the subject an inhibitory RNA polynucleotide described herein that has partial complementarity to the target gene.

As used herein, the terms “subject,” “individual,” and “patient” are used interchangeably herein to refer to a vertebrate, preferably a mammal, more preferably a human. Mammals include, but are not limited to, murines, rats, simians, humans, farm animals, sport animals, and pets.

### III. Inhibitory RNA Polynucleotide and Methods of the Disclosure

The disclosure provides inhibitory RNA polynucleotides comprising between 15 and 30 nucleotides that are designed to be partially complementary to an equal length portion of a target gene. As described herein, the inventors applied high-throughput methods to measure the association kinetics, equilibrium binding energies, and single-turnover cleavage rates of the RNA-Induced Silencing Complex (RISC) to find that RISC readily tolerates specific nucleotide mismatches between the inhibitory RNA polynucleotide and its target gene. In some embodiments, the nucleotide mismatches enhance the rate of target gene cleavage. In some embodiments, the nucleotide mismatches decrease the rate of target gene cleavage. The compositions and methods disclosed herein provide useful strategies for designing inhibitory RNA polynucleotides.

The disclosure provides an inhibitory RNA polynucleotide comprising between 15 and 30 (e.g., between 15 and 28, between 15 and 26, between 15 and 24, between 15 and 22, between 15 and 20, between 15 and 18, between 15 and 16, between 16 and 30, between 18 and 30, between 20 and 30, between 22 and 30, between 24 and 30, between 26 and 30, between 28 and 30, 15, 16, 17, 18, 19, 20, 21, 22, 23, 24, 25, 26, 27, 28, 29, or 30) nucleotides in which there is at least one mismatched nucleotide at position 5, 7, 8, 12, 16, 17, 18, 19, 20, or 21, and the inhibitory RNA polynucleotide guides an RNA-induced silencing complex (RISC) to cleave the target gene. A mismatched nucleotide in an inhibitory RNA polynucleotide described herein is a nucleotide at a specific position in the inhibitory RNA polynucleotide that does not engage in Watson-Crick base pairing with a nucleotide at the corresponding position in the target gene when the inhibitory RNA polynucleotide hybridizes to an equal length portion of the target gene. The mismatched nucleotide does not prevent the binding between the inhibitory RNA polynucleotide and its target gene. In some embodiments, the inhibitory RNA polynucleotide has one mismatched nucleotide at position 5. In some embodiments, the inhibitory RNA polynucleotide has one mismatched nucleotide at position 7. In some embodiments, the inhibitory RNA polynucleotide has one mismatched nucleotide at position 8. In some embodiments, the inhibitory RNA polynucleotide has one mismatched nucleotide at position 12. In some embodiments, the inhibitory RNA polynucleotide has one mismatched nucleotide at position 16. In some embodiments, the inhibitory RNA polynucleotide has one mismatched nucleotide at position 17. In some embodiments, the inhibitory RNA polynucleotide has one mismatched nucleotide at position 18. In some embodiments, the inhibitory RNA polynucleotide has one mismatched nucleotide at position 19. In some embodiments, the inhibitory RNA polynucleotide has one mismatched nucleotide at position 20. In some embodiments, the inhibitory RNA polynucleotide has one mismatched nucleotide at position 21.

The inhibitory RNA polynucleotide described herein can have at least two mismatched nucleotides at positions selected from positions 5, 7, 8, 12, 15, 16, 17, 18, 19, 20, and 21 (e.g., positions 15, 16, 17, 18, 19, 20, and 21). In certain embodiments, the inhibitory RNA polynucleotide has two mismatched nucleotides in which a first mismatched nucleotide is at position 12 and a second mismatched nucleotide is at position 5, 7, 8, 15, 16, 17, 18, 19, 20, or 21. In particular embodiments, the inhibitory RNA polynucleotide has a first mismatched nucleotide at position 12 and a second mismatched nucleotide at position 5. In particular embodiments, the inhibitory RNA polynucleotide has a first mismatched nucleotide at position 12 and a second mismatched nucleotide at position 7. In particular embodiments, the inhibitory RNA polynucleotide has a first mismatched nucleotide at position 12 and a second mismatched nucleotide at position 8. In particular embodiments, the inhibitory RNA polynucleotide has a first mismatched nucleotide at position 12 and a second mismatched nucleotide at position 15. In particular embodiments, the inhibitory RNA polynucleotide has a first mismatched nucleotide at position 12 and a second mismatched nucleotide at position 16. In particular embodiments, the inhibitory RNA polynucleotide has a first mismatched nucleotide at position 12 and a second mismatched nucleotide at position 17. In particular embodiments, the inhibitory RNA polynucleotide has a first mismatched nucleotide at position 12 and a second mismatched nucleotide at position 18. In particular embodiments, the inhibitory RNA polynucleotide has a first mismatched nucleotide at position 12 and a second mismatched nucleotide at position 19. In particular embodiments, the inhibitory RNA polynucleotide has a first mismatched nucleotide at position 12 and a second mismatched nucleotide at position 20. In particular embodiments, the inhibitory RNA polynucleotide has a first mismatched nucleotide at position 12 and a second mismatched nucleotide at position 21.

In certain embodiments, the inhibitory RNA polynucleotide has two mismatched nucleotides in which a first mismatched nucleotide is at position 18 and a second mismatched nucleotide is at position 5, 7, 8, 12, 15, 16, 17, 19, 20, or 21. In particular embodiments, the inhibitory RNA polynucleotide has a first mismatched nucleotide at position 18 and a second mismatched nucleotide at position 5. In particular embodiments, the inhibitory RNA polynucleotide has a first mismatched nucleotide at position 18 and a second mismatched nucleotide at position 7. In particular embodiments, the inhibitory RNA polynucleotide has a first mismatched nucleotide at position 18 and a second mismatched nucleotide at position 8. In particular embodiments, the inhibitory RNA polynucleotide has a first mismatched nucleotide at position 18 and a second mismatched nucleotide at position 12. In particular embodiments, the inhibitory RNA polynucleotide has a first mismatched nucleotide at position 18 and a second mismatched nucleotide at position 15. In particular embodiments, the inhibitory RNA polynucleotide has a first mismatched nucleotide at position 18 and a second mismatched nucleotide at position 16. In particular embodiments, the inhibitory RNA polynucleotide has a first mismatched nucleotide at position 18 and a second mismatched nucleotide at position 17. In particular embodiments, the inhibitory RNA polynucleotide has a first mismatched nucleotide at position 18 and a second mismatched nucleotide at position 19. In particular embodiments, the inhibitory RNA polynucleotide has a first mismatched nucleotide at position 18 and a second mismatched nucleotide at position 20. In particular embodiments, the inhibitory RNA polynucleotide has a first mismatched nucleotide at position 18 and a second mismatched nucleotide at position 21.

In some embodiments, the inhibitory RNA polynucleotide has mismatched nucleotides at positions 15, 16, 17, 18, 19, 20, and 21 (e.g., positions 17, 18, 19, 20, and 21).

As disclosed herein, the inhibitory RNA polynucleotides having one or more mismatched nucleotides (i.e., partial complementarity) can guide the RISC to cleave the target gene at a faster cleavage rate than the corresponding cleavage rate of RISC when RISC is guided by a corresponding inhibitory RNA polynucleotide having complete complementarity to the equal length portion of the target gene. In certain embodiments, the target gene cleavage rate of RISC when loaded with an inhibitory RNA polynucleotide described herein is at least 5% (e.g., at least 6%, 7%, 8%, 9%, 10%, 15%, 20%, 25%, 30%, 35%, 40%, 45%, or 50%) faster than the cleavage rate of RISC when loaded with an inhibitory RNA polynucleotide having complete complementarity to the target gene.

As described further herein, an inhibitory RNA polynucleotide can be in the form of an miRNA, siRNA, shRNA, or aiRNA. The inhibitory RNA polynucleotide can also be a single-stranded polynucleotide or a double-stranded polynucleotide.

An inhibitory RNA polynucleotide described herein can be used in methods of increasing or decreasing the cleavage rate of an RNA-induced silencing complex (RISC) in cleaving a target gene. To increase the cleavage rate of RISC, at least one mismatched nucleotide (e.g., at least one mismatched nucleotide at position 5, 7, 8, 12, 16, 17, 18, 19, 20, or 21) can be introduced to an inhibitory RNA polynucleotide comprising between 15 and 30 nucleotides. In certain embodiments, to increase the cleavage rate of RISC, at least two mismatched nucleotides at positions selected from positions 5, 7, 8, 12, 15, 16, 17, 18, 19, 20, and 21 can be introduced to an inhibitory RNA polynucleotide comprising between 15 and 30 nucleotides. To synthesize an inhibitory RNA polynucleotide with an increased cleavage rate for a target gene, the method includes: (a) providing a sequence of the target gene; (b) selecting a portion of the sequence of the target gene where the inhibitory RNA polynucleotide binds; (c) selecting at least one position from positions 5, 7, 8, 12, 16, 17, 18, 19, 20, and 21 of the inhibitory RNA polynucleotide to introduce a mismatched nucleotide at the position; and (c) introducing the mismatched nucleotide at the selected position of the inhibitory RNA polynucleotide during synthesis of the inhibitory RNA polynucleotide, in which the inhibitory RNA polynucleotide is partially complementary to an equal length portion of the target gene, and in which an RISC is guided by the inhibitory RNA polynucleotide to bind and cleave the target gene at a faster cleavage rate than the corresponding cleavage rate of RISC when RISC is guided by a corresponding inhibitory RNA polynucleotide having complete complementarity to the equal length portion of the target gene.

In some embodiments, mismatched nucleotides at different positions in an inhibitory RNA polynucleotide can be introduced to decrease the cleavage rate of the inhibitory RNA polynucleotide. In particular embodiments, the disclosure provides a method of decreasing the cleavage rate of an RNA-induced silencing complex (RISC) in cleaving a target gene, comprising introducing at least two mismatched nucleotides at positions selected from positions 9, 10, 11, and 13 to an inhibitory RNA polynucleotide comprising between 15 and 30 nucleotides. In this embodiment, the inhibitory RNA polynucleotide comprising at least two mismatched polynucleotides can decrease the cleavage rate of RISC (e.g., decrease the cleavage rate of RISC by at least 5% (e.g., at least 6%, 7%, 8%, 9%, 10%, 15%, 20%, 25%, 30%, 35%, 40%, 45%, or 50%)) relative to the cleavage or RISC when loaded with a corresponding inhibitory RNA polynucleotide having complete complementarity to the equal length portion of the target gene.

Further, the inhibitory RNA polynucleotides described herein that have partial complementarity to the target gene can be used to decrease the expression level of the target gene in a cell. Specifically, the inhibitory RNA polynucleotides can be used to treat a disease in a subject in need thereof, such as a disease that is caused, related to, or aggravated by the target gene.

### IV. Nucleic Acid Therapeutics

The inhibitory RNA polynucleotides described herein can be used to treat a disease, e.g., a genetic disease, where the genetic sequence of a particular gene is known to cause the disease. An inhibitory RNA polynucleotide described herein can be synthesized to target the disease-causing gene to inactivate it and/or to lower its expression level. An inhibitory RNA polynucleotide described herein can contain naturally-occurring bases, non-naturally-occurring bases, sugars, and backbone linkages. The inhibitory RNA polynucleotide can be of various lengths, e.g., between 15 and 30 nucleotides (e.g., 15, 16, 17, 18, 19, 20, 21, 22, 23, 24, 25, 26, 27, 28, 29, or 30 nucleotides). In further embodiments, the inhibitory RNA polynucleotide can be single-stranded or double-stranded. The inhibitory RNA polynucleotide can specifically hybridize to or is complementary (e.g., partially complementary) to a target gene, such that stable and specific binding occurs between the inhibitory RNA polynucleotide and the target gene. The binding of the inhibitory RNA polynucleotide to the target gene can interfere with the normal function of the target gene to cause a loss of utility or expression therefrom, and there is a sufficient degree of complementarity between the inhibitory RNA polynucleotide and the target gene to avoid non-specific binding of the inhibitory RNA polynucleotide to non-target sequences.

miRNA

Furthermore, the inhibitory RNA polynucleotides described herein can be a microRNA, which is a single-stranded RNA molecule of about 21-23 nucleotides (e.g., 21, 22, or 23 nucleotides) in length. miRNAs are encoded by genes from whose DNA they are transcribed, but miRNAs are not translated into protein (non-coding RNA); instead, each primary transcript (a pri-miRNA) is processed into a short stem-loop structure called a pre-miRNA and finally into a functional mature miRNA. Mature miRNA molecules are either partially or completely complementary to one or more messenger RNA (mRNA) molecules.

miRNAs are first transcribed as primary transcripts or pri-miRNA with a cap and poly-A tail and processed to short, nucleotide stem-loop structures known as pre-miRNA in the cell nucleus by a protein complex known as the Microprocessor complex, consisting of the nuclease Drosha and the double-stranded RNA binding protein Pasha (Denli et al., Nature, 432:231-235,2004). These pre-miRNAs are then processed to mature miRNAs in the cytoplasm by interaction with the endonuclease Dicer, which also initiates the formation of the RNA-induced silencing complex (RISC) (Bernstein et al., Nature, 409:363-366, 2001. Either the sense strand or antisense strand of DNA can function as templates to give rise to miRNA. When Dicer cleaves the pre-miRNA stem-loop, two complementary short RNA molecules are formed, but only one is integrated into the RISC complex. This strand is known as the guide strand and is selected by the argonaute protein, which is the catalytically active RNase in the RISC complex, on the basis of the stability of the 5′ end (Preall et al., Curr. Biol., 16:530-535, 2006). The remaining strand, known as the anti-guide or passenger strand, is degraded as a RISC complex substrate). After integration into the active RISC complex, miRNAs base pair with their complementary mRNA molecules and induce target mRNA degradation and/or translational silencing.

Mammalian miRNA molecules are usually complementary to a site in the 3′ UTR of the target mRNA sequence. In some embodiments, the annealing of the miRNA to the target mRNA inhibits protein translation by blocking the protein translation machinery. In some embodiments, the annealing of the miRNA to the target mRNA facilitates the cleavage and degradation of the target mRNA through a process similar to RNA interference (RNAi).

siRNA

The inhibitory RNA polynucleotides described herein can be a small interfering RNA (siRNA), which refers to a double-stranded RNA with the two complementary strands each having between 15 and 20 nucleotides (e.g., 15, 16, 17, 18, 19, or 20 nucleotides). In some embodiments, the two strands of an siRNA molecule can each have a 3′-end overhang of two or three nucleotides. In an siRNA molecule, one strand (e.g., the antisense strand) is guiding and complementary (e.g., partially complementary) to the target gene.

Suitable siRNA sequences can be identified using methods known in the art. For example, prediction algorithms that predict potential siRNA-targets based upon complementary DNA sequences in the target genes are available in the art. TargetScanHuman, for example, is a comprehensive web resource for inhibitory RNA-target predictions, and uses an algorithm that incorporates current biological knowledge of inhibitory RNA-target rules including seed-match model, evolutionary conservation, and free binding energy (Li and Zhang, Wiley Interdiscip Rev RNA 6:435-452, 2015 and Agarwal et al., Elife 4, 2015). The target sites predicted by TargetScanHuman are scored for likelihood of mRNA down-regulation using context scores (CS), a regression model that is trained on sequence and contextual features of the predicted inhibitory RNA::mRNA duplex. In large-scale evaluations, TargetScanHuman has been competitive with other target prediction methods in identifying target genes and predicting the extent of their down-regulation at the mRNA or protein levels. In some embodiments, to further enhance silencing efficiency of the siRNA sequences, potential siRNA sequences may be analyzed to identify sites that do not contain regions of homology to other coding sequences, e.g., in the target cell or organism.

Once a potential siRNA sequence has been identified, a complementary sequence (i.e., an antisense strand sequence) can be designed. A potential siRNA sequence can also be analyzed using a variety of criteria known in the art. For example. to enhance their silencing efficiency, the siRNA sequences may be analyzed by a rational design algorithm to identify sequences that have one or more of the following features: (1) G/C content of about 25% to about 60% G/C; (2) at least 3 A/Us at positions 15-19 of the sense strand; (3) no internal repeats; (4) an A at position 19 of the sense strand; (5) an A at position 3 of the sense strand; (6) a U at position 10 of the sense strand; (7) no G/C at position 19 of the sense strand; and (8) no G at position 13 of the sense strand. The siRNA design tools that incorporate algorithms that assign suitable values of each of these features and are useful for selection of the siRNA are available in the art. One of skill in the art will appreciate that sequences with one or more of the foregoing characteristics may be selected for farther analysis and testing as potential siRNA sequences.

In some embodiments, potential siRNA sequences may be further analyzed based on siRNA duplex asymmetry as described in, e.g., Khvorova et al., Cell 115:209-216, 2003 and Schwarz et al., Cell 115:199-208, 2003. In other embodiments, potential siRNA sequences may be further analyzed based on secondary structure at the target site as described in, e.g., Luo et al., Biophys. Res. Commun. 318:303-310, 2004. For example, secondary structure at the target site can be modeled using available techniques in the art, e.g., Mfold algorithm to select siRNA sequences which favor accessibility at the target site where less secondary structure in the form of base-pairing and stem-loops is present.

shRNA

The inhibitory RNA polynucleotides described herein can also be a small hairpin RNA or short hairpin RNA (shRNA), which is a short RNA sequence that makes a tight hairpin turn that can be used to silence gene expression via RNA interference. The shRNA hairpin structure is cleaved by the cellular machinery into siRNA, which is then bound to the RNA-induced silencing complex (RISC). In some embodiments, shRNAs can be between 15 to 60 nucleotides (e.g., 15, 20, 25, 30, 35, 40, 45, 50, 55, or 60 nucleotides) in length. Non-limiting examples of shRNA include a double-stranded polynucleotide molecule assembled from a single-stranded molecule, in which the sense and antisense regions are linked by a nucleic acid-based or non-nucleic acid-based linker; and a double-stranded polynucleotide molecule with a hairpin secondary structure having self-complementary sense and antisense regions.

aiRNA

Furthermore, similar to siRNA, asymmetrical interfering RNA (aiRNA) can recruit the RNA-induced silencing complex (RISC) and lead to effective silencing of genes in mammalian cells by mediating sequence-specific cleavage of the target sequence between nucleotide 10 and 11 relative to the 5′ end nucleotide. Typically, an aiRNA molecule comprises a short RNA duplex having a sense strand and an antisense strand, wherein the duplex contains overhangs at the 3′ and 5′ ends of the antisense strand. The aiRNA is generally asymmetric because the sense strand is shorter on both ends when compared to the complementary antisense strand.

In some aspects, aiRNA molecules may be designed, synthesized, and annealed under conditions similar to those used for siRNA molecules. As a non-limiting example, aiRNA sequences may be selected and generated using the methods described above for selecting siRNA sequences. In another example, aiRNA duplexes of various lengths (e.g., about 10-25, 12-20, 12-19, 12-18, 13-17, or 14-17 base pairs, more typically 12, 13, 14, 15, 16, 17, 18, 19, or 20 base pairs) may be designed with overhangs at the 3′ and 5′ ends of the antisense strand to target an mRNA of interest. In certain instances, the sense strand of the aiRNA molecule is about 10-25, 12-20, 12-19, 12-18, 13-17, or 14-17 nucleotides in length, more typically 12, 13, 14, 15, 16, 17, 18, 19, or 20 nucleotides in length. In certain other instances, the antisense strand of the aiRNA molecule is about 15-30 (e.g., 15, 16, 17, 18, 19, 20, 21, 22, 23, 24, 25, 26, 27, 28, 29, or 30 nucleotides) nucleotides in length. In some embodiments, the 5′ antisense overhang contains one, two, three, four, or more non-targeting nucleotides (e.g., AA, UU, dTdT, etc.). In other embodiments, the 3′ antisense overhang contains one, two, three, four, or more non-targeting nucleotides (e.g., AA, UU, dTdT, etc.).

In any of the RNA therapeutics described above, an inhibitory RNA polynucleotide of the disclosure can further include modifications that improve the pharmacokinetics of the polynucleotide, i.e., modification to increase half-life. Possible modifications include, but are not limited to, modifications on one or more sugar residues, modifications on one or more internucleoside linkages, and modifications on one or more nucleobases. Modified sugar residues can include, e.g., a pentofuranosyl sugar, a locked sugar, and an unlocked sugar. Modified internucleoside linkages can include, e.g., a phosphorothioate linkage, a phosphorodithioate linkage, and a thiophosphoramidate linkage. Modified nucleobases can include, e.g., hypoxanthine, xanthine, 7-methylguanine, 5,6-dihydrouracil, 5-methylcytosine, and 5-hydroxymethylcytosine.

### V. Pharmaceutical Compositions, Preparation, and Delivery

The disclosure features pharmaceutical compositions that include an inhibitory RNA polynucleotide described herein and a pharmaceutically acceptable carrier. In some embodiments of the pharmaceutical composition, the inhibitory RNA polynucleotide is encapsulated in a nanoparticle (e.g., a liposome (e.g., a polyethylene glycol (PEG) liposome)). In some embodiments, the pharmaceutical composition including the inhibitory RNA polynucleotide may be formulated for intravenous delivery using a nanoparticle (e.g., a PEG liposome). Further, the disclosure also provides kits containing an inhibitory RNA polynucleotide described herein and a nanoparticle (e.g., a PEG liposome). The inhibitory RNA polynucleotide and the nanoparticle may be provided in separate containers or compartments. The inhibitory RNA polynucleotide may be packaged into the nanoparticle (e.g., a PEG liposome) prior to administration (e.g., intravenous administration).

Nanoparticles used to package and deliver an inhibitory RNA polynucleotide as described herein may be lipid-based nanoparticles or polymer-based nanoparticles. Lipid-based nanoparticles are constructed using lipid components and include a vesicle wall containing a single- or double-lipid layer that surrounds a cavity. Examples of lipid-based nanoparticles include, but are not limited to, e.g., liposomes, exosomes, and micelles. Polymer-based nanoparticles are constructed mainly using amphiphilic molecules and amphiphilic polymers, e.g., dodecyltrimethylammonium bromide, sodium dodecylsulfate, betaine, alkyl glycoside, pentaethyllene glycol monododecyl ether, phosphatidylcholine, sodium polyacrylate, poly-N-isopropylacrylamide, poloxamer, and cellulose. Polymer-based nanoparticles may be constructed using one or more types of these amphiphilic molecules and amphiphilic polymers. In addition to the inhibitory RNA polynucleotide, the pharmaceutical compositions may contain one or more pharmaceutically acceptable carriers or excipients, which can be formulated by methods known to those skilled in the art. In some embodiments, a pharmaceutical composition of the present disclosure includes an inhibitory RNA polynucleotide in a therapeutically effective amount. In certain embodiments, the therapeutically effective amount of the inhibitory RNA polynucleotide is sufficient to treat the disease and/or sufficient to decrease the expression level of a target gene in the disease. Determination of a therapeutically effective amount is within the capability of those skilled in the art.

Liposome Delivery

In some embodiments, an inhibitory RNA polynucleotide described herein may be loaded or packaged in liposomes (e.g., polyethylene glycol 2000 (PEG)-liposomes) for intravenous delivery. The PEG-liposome based drug delivery system has been approved by FDA for human use, and has several advantages: (1) it is biodegradable and does not cause toxicity or inflammatory response, (2) the conjugated complexes are stable in serum, and can improve the in vivo half-life of the inhibitory RNA polynucleotide and enhance the entry of the inhibitory RNA polynucleotide into cells, and (3) it can produce a transient elevation of the inhibitory RNA polynucleotide after administration. In some embodiments, it is also essential to modulate the inhibitory RNA polynucleotide transiently to avoid the potential side effects caused by long-term overexpression.

In some embodiments, suitable liposomes may be formed from standard vesicle-forming lipids, which generally include neutral or negatively charged phospholipids and a sterol, such as cholesterol. Embodiments of the disclosure features the package and delivery of the miR or mimic thereof in surface-modified liposomes containing PEG lipids (PEG-modified liposomes). These formulations increase the circulation and accumulation of the miR-containing liposome in target tissues. The long-circulating liposomes are protected from nuclease degradation and enhance the pharmacokinetics and pharmacodynamics of the miR or mimic thereof.

The selection of lipids is generally guided by consideration of factors such as desired liposome size and half-life of liposome in the bloodstream. Further considered are liposomes modified so as to avoid clearance by the mononuclear macrophages and reticuloendothelial systems, for example, having opsonization-inhibition moieties bound to the surface of the liposome structures. Opsonization-inhibition moieties are large hydrophilic polymers bound to the liposome membrane, for example, polyethylene glycol or polypropylene glycol and derivatives thereof, e.g., methoxy derivatives or stearates, or also synthetic polymers such as polyacrylamide or polyvinyl-pyrrolidone, linear, branched, or dendrimeric polyamidoamines, polyacrylic acids, polyalcohols, e.g., polyvinyl alcohols and polyxylitol, and gangliosides. In some embodiments, opsonization-inhibition moieties may be polyethylene glycol or polypropylene glycol and derivatives thereof giving rise to “pegylated liposomes,” resulting in stable nucleic acid-lipid particles.

Amphoteric liposomes are another class of liposomes that may be used to delivery the miR or mimic thereof. Amphoteric liposomes are pH dependent charge-transitioning particles that can provide for the delivery of a nucleic acid payload to cells either by local or systemic administration. Amphoteric liposomes can be designed to release their nucleic acid payload within the target cell where the nucleic acid can then engage a number of biological pathways, and thereby exert a therapeutic effect.

Exosome Delivery

In some embodiments, the inhibitory RNA polynucleotide may be loaded or packaged in exosomes that specifically target a cell type, tissue, or organ to be treated. Exosomes are small membrane-bound vesicles of endocytic origin that are released into the extracellular environment following fusion of mutivesicular bodies with the plasma membrane. Exosome production has been described for many immune cells including B cells, T cells, and dendritic cells. Techniques used to load a therapeutic compound (i.e., an miR or mimic thereof) into exosomes are known in the art and described in, e.g., U.S. Patent Publication Nos. US 20130053426 and US 20140348904, and International Patent Publication No. WO 2015002956, which are incorporated herein by reference. In some embodiments, therapeutic compounds may be loaded into exosomes by electroporation or the use of a transfection reagent (i.e., cationic liposomes).

In some embodiments, an exosome-producing cell can be engineered to produce the exosome and load it with the therapeutic compound. For example, exosomes may be loaded by transforming or transfecting an exosome-producing host cell with a genetic construct that expresses the therapeutic compound, such that the therapeutic compound is taken up into the exosomes as the exosomes are produced by the host cell. In some embodiments, an exosome-targeted protein in the exosome-producing cell may bind (i.e., non-covalently) to the therapeutic compound. Various targeting moieties may be introduced into exosomes, so that the exosomes can be targeted to a selected cell type, tissue, or organ. Targeting moieties may bind to cell-surface receptors or other cell-surface proteins or peptides that are specific to the targeted cell type, tissue, or organ. In some embodiments, exosomes have a targeting moiety expressed on their surface. In some embodiments, the targeting moiety expressed on the surface of exosomes is fused to an exosomal transmembrane protein. Techniques of introducing targeting moieties to exosomes are known in the art and described in, e.g., U.S. Patent Publication Nos. US 20130053426 and US 20140348904, and International Patent Publication No. WO 2015002956, which are incorporated herein by reference.

Preparation

The inhibitory RNA polynucleotide described herein may be mixed with pharmaceutically acceptable active and/or inert substances for the preparation of pharmaceutical compositions. Compositions and methods for the formulation of pharmaceutical compositions are dependent upon a number of criteria, including, but not limited to, route of administration, extent of disease, or dose to be administered. An inhibitory RNA polynucleotide used in methods of the disclosure may be utilized in pharmaceutical compositions by combining the miR or mimic thereof with a suitable pharmaceutically acceptable diluent or carrier. A pharmaceutically acceptable diluent includes phosphate-buffered saline (PBS). PBS is a diluent suitable for use in compositions to be delivered parenterally (e.g., intravenously).

In some embodiments, a pharmaceutical composition is prepared for administration by injection (e.g., intravenous, subcutaneous, intramuscular, etc.). In some embodiments, a pharmaceutical composition includes a carrier and is formulated in aqueous solution, such as water or physiologically compatible buffers such as PBS, Hank's solution, Ringer's solution, or physiological saline buffer. Examples of solvents suitable for use in pharmaceutical compositions for injection include, but are not limited to, lipophilic solvents and fatty oils, such as sesame oil, and synthetic fatty acid esters, such as ethyl oleate or triglycerides. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. In some embodiments, carriers and excipients are selected from water, salt solutions, alcohol, polyethylene glycols, gelatin, lactose, amylase, magnesium stearate, talc, silicic acid, viscous paraffin, hydroxymethylcellulosem, and polyvinylpyrrolidone.

In some embodiments, a pharmaceutical composition of the present disclosure is prepared using known techniques, including, but not limited to mixing, dissolving, granulating, dragee-making, levigating, emulsifying, encapsulating, entrapping, and tabletting processes. In some embodiments, a pharmaceutical composition of the present disclosure is a liquid (e.g., a suspension, elixir and/or solution). In some embodiments, a liquid pharmaceutical composition is prepared using ingredients known in the art, including, but not limited to, water, glycols, oils, alcohols, flavoring agents, preservatives, and coloring agents. In some embodiments, a pharmaceutical composition of the present disclosure is a solid (e.g., a powder, tablet, and/or capsule). In some embodiments, a solid pharmaceutical composition is prepared using ingredients known in the art, including, but not limited to, starches, sugars, diluents, granulating agents, lubricants, binders, and disintegrating agents.

In some embodiments, the inhibitory RNA polynucleotide may be reconstituted with a suitable diluent, e.g., sterile water for injection. The reconstituted product may be administered as an intravenous infusion after dilution into saline. In some embodiments, the pH of the pharmaceutical composition may be adjusted to pH 7.0-9.0 with acid or base during preparation.

In some embodiments, a pharmaceutical composition is prepared for gene therapy. In some embodiments, the pharmaceutical composition for gene therapy is in an acceptable diluent, or includes a slow release matrix in which the gene delivery vehicle is imbedded. Vectors that may be used as in vivo gene delivery vehicle include, but are not limited to, retroviral vectors, adenoviral vectors, poxviral vectors (e.g., vaccinia viral vectors, such as Modified Vaccinia Ankara), adeno-associated viral vectors, and alphaviral vectors.

### VI. Routes, Dosage, and Administration

Pharmaceutical compositions including an inhibitory RNA polynucleotide described herein may be formulated for parenteral administration, e.g., intravenous administration, subcutaneous administration, intramuscular administration, intraarterial administration, intrathecal administration, or intraperitoneal administration. In particular embodiments, the pharmaceutical composition may be formulated for intravenous administration. For injectable formulations, various effective pharmaceutical carriers are known in the art, see, e.g., ASHP Handbook on Injectable Drugs, Trissel, 18th ed. (2014). Other administration routes include, but are not limited to, oral, rectal, transmucosal, intestinal, enteral, topical, suppository, through inhalation, intranasal, and intraocular administration.

In some embodiments, administration may include a single dose or multiple doses. In some embodiments, pharmaceutical compositions for injection are presented in unit dosage form, e.g., in ampoules or in multi-dose containers. In particular embodiments, pharmaceutical compositions containing liposomes (e.g., PEG liposomes) packaged with the inhibitory RNA polynucleotide may be intravenously administered to the subject in a single dose or multiple doses.

In some embodiments, a pharmaceutical composition described herein is administered in the form of a dosage unit (e.g., bolus). In some embodiments, a pharmaceutical compositions includes an inhibitory RNA polynucleotide in a dose selected from 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 270 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, and 800 mg. In some embodiments, a pharmaceutical composition described herein includes a dose of an inhibitory RNA polynucleotide selected from 25 mg, 50 mg, 75 mg, 100 mg, 150 mg, 200 mg, 250 mg, 300 mg, 350 mg, 400 mg, 500 mg, 600 mg, 700 mg, and 800 mg. In some embodiments, a pharmaceutical composition includes a dose of the inhibitory RNA polynucleotide selected from 50 mg, 100 mg, 150 mg, 200 mg, 250 mg, 300 mg, and 400 mg. In some embodiments, a pharmaceutical composition includes an inhibitory RNA polynucleotide in a dose ranging from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 100, 150, 200, 250, 300, 350, 400, 450, or 500 mg/kg) and, in a more specific embodiment, about 0.1 to about 50 mg/kg and, in a more specific embodiment, about 1 to about 5 mg/kg.

The pharmaceutical compositions are administered in a manner compatible with the dosage formulation and in such amount as is therapeutically effective to result in an improvement or remediation of the symptoms. In some embodiments, the dose is administered at intervals ranging from more than once per day, once per day, once per week, twice per week, three times per week, four times per week, five times per week, six times per week, once per month to once per three months, for as long as needed to sustain the desired effect. The timing between administrations may decrease as the medical condition improves or increase as the health of the patient declines. The dosage may be adapted by the physician in accordance with conventional factors such as the extent of the disease and different parameters of the subject.

### EXAMPLES

### Example 1

### High-Throughput Measurement of RISC Binding

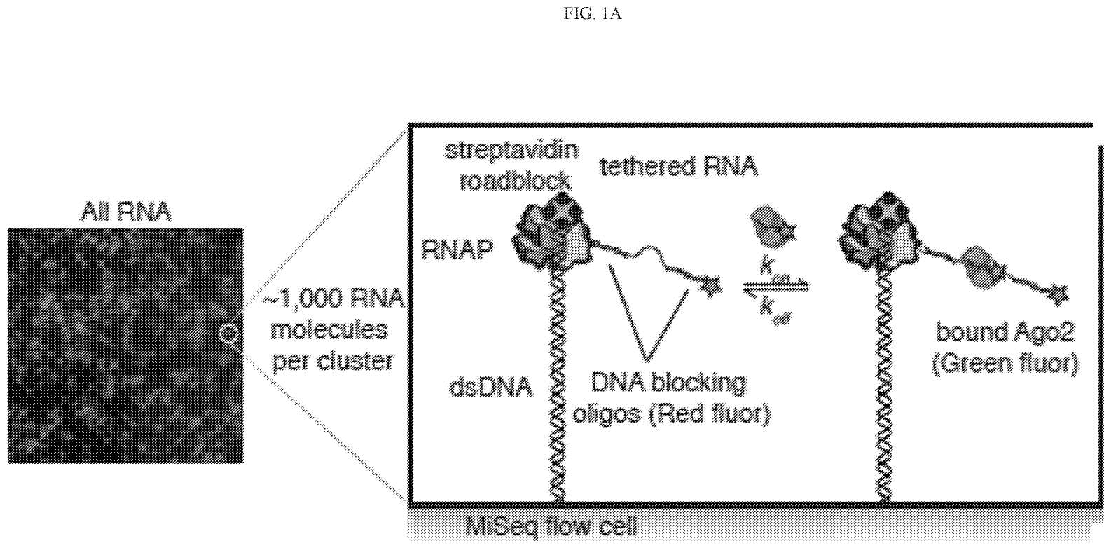

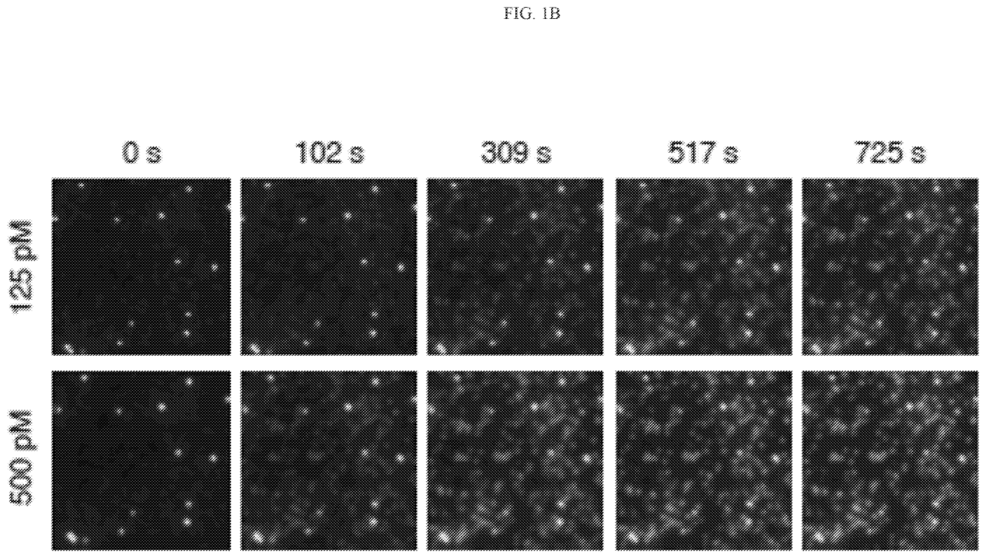

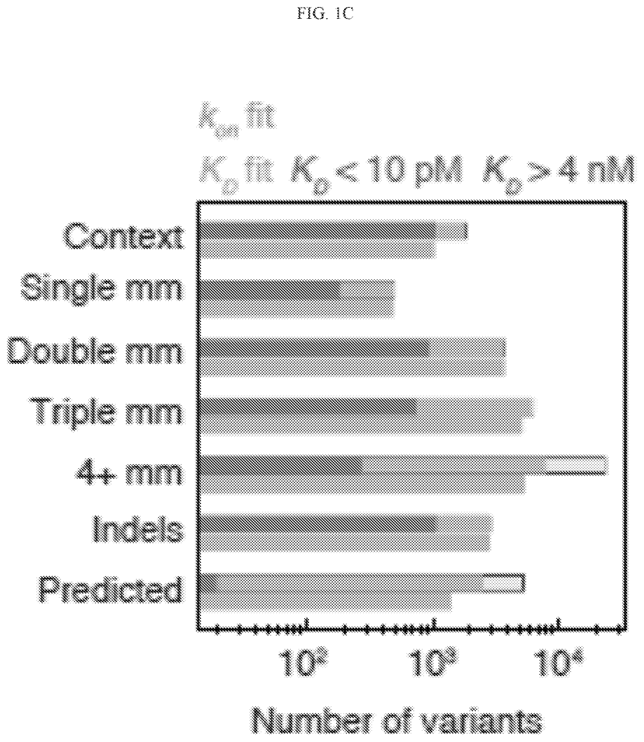

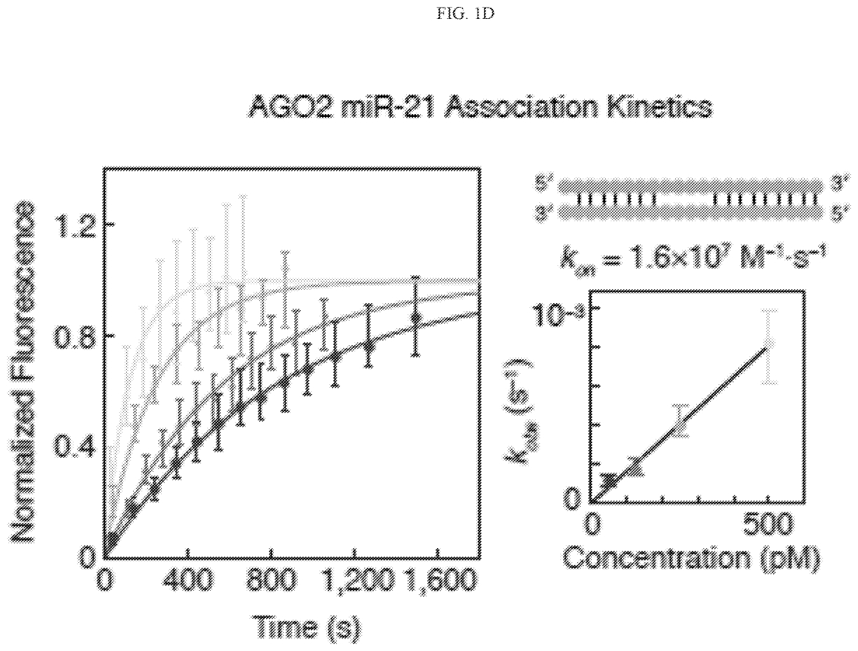

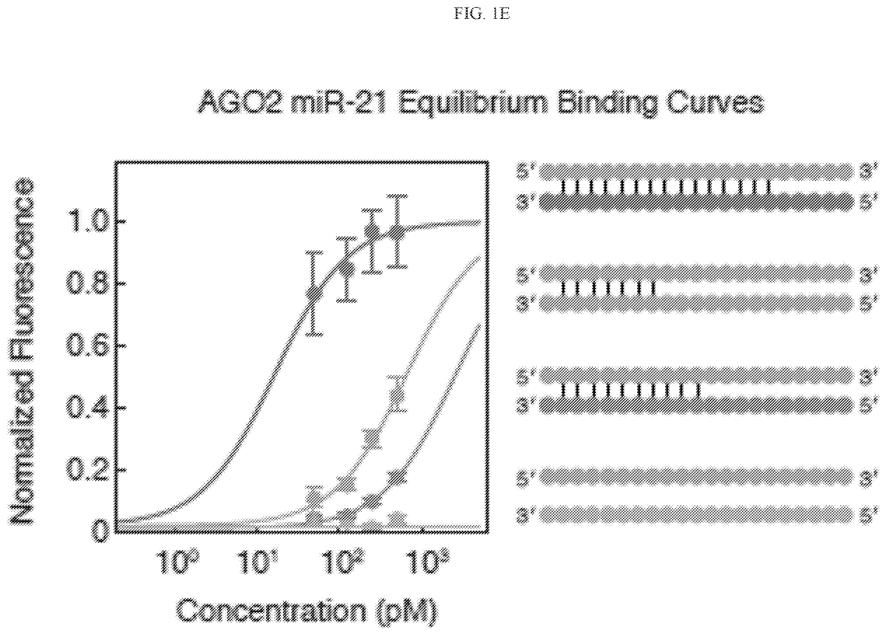

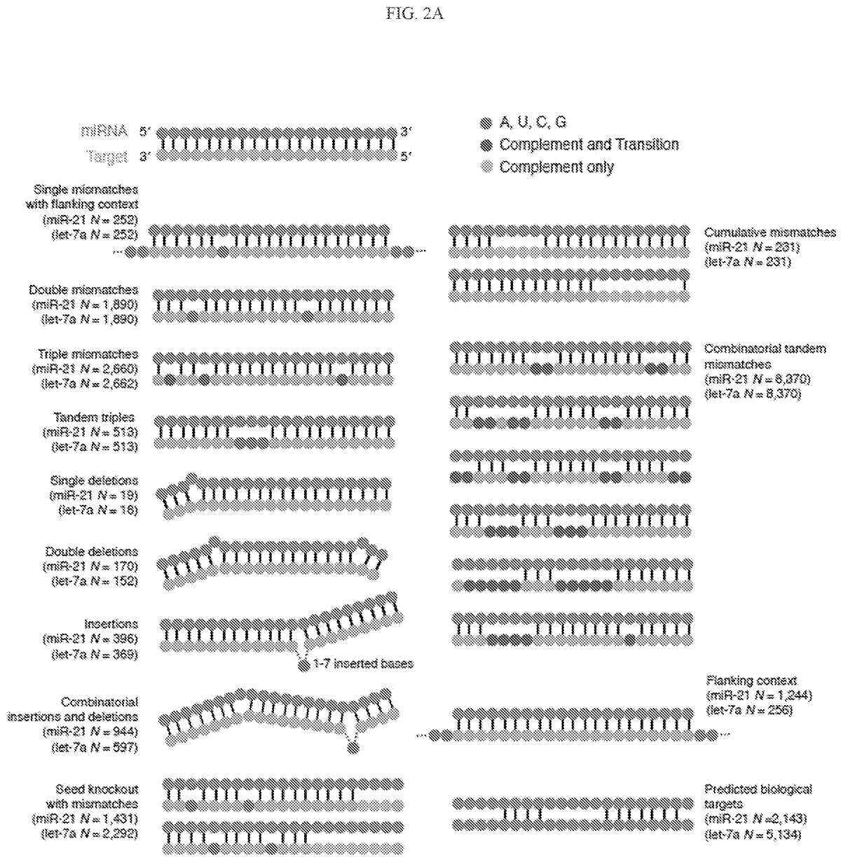

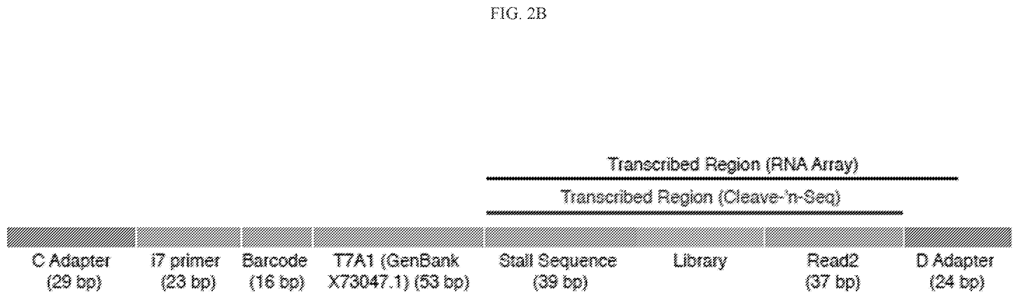

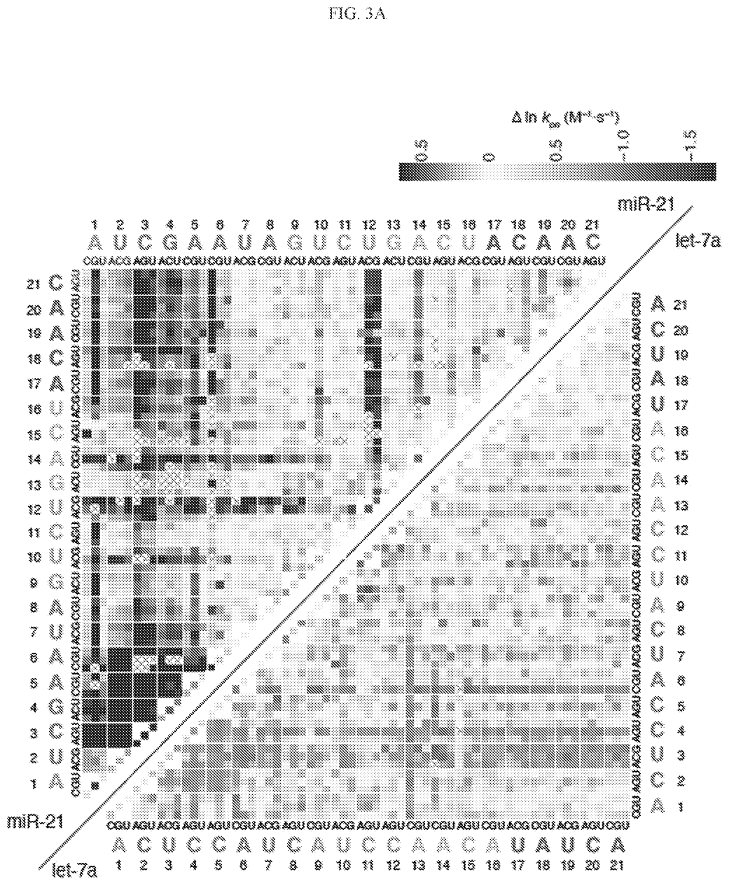

To define the sequence determinants of RISC binding, libraries comprising ˜20,000 distinct RNA targets per guide were designed. The libraries include all singly and doubly mismatched targets; a subset of triple and higher-order (≥4) mismatched targets; all single and double deletions; homopolymer insertions up to 7 nucleotides long; as well as targets predicted by TargetScan, Diana-microT, miRanda-mirSVR, and PicTar2, and targets identified by CLASH (crosslinking, ligation, and sequencing of hybrids; FIG. 1C and FIG. 2A) (Krek et al., 2005; Betel et al., 2010; Reczko et al., 2012; Helwak et al., 2013; Khorshid et al., 2013; Agarwal et al., 2015). Target libraries were synthesized as DNA oligonucleotides, sequenced on an Illumina MiSeq, and transcribed in situ to generate clusters of RNA tethered to DNA templates of known sequence (FIGS. 1A and 1B) (Buenrostro et al., 2014; She et al., 2017; Denny et al., 2018). To eliminate potential secondary structures and cryptic binding sites, DNA oligonucleotides were annealed to the RNA flanking the target sequence. Binding kinetics were measured using catalytically inactive D669A mutant AGO2 and cleavage was measured using wild-type AGO2. Mouse AGO2 was loaded with 3′ Alexa555-labeled let-7a, a well-studied seed-driven miRNA, or miR-21, a miRNA that requires 3′ supplemental pairing for its highest affinity binding (Salomon et al., 2015). RISC was continuously flowed into the MiSeq chip at multiple concentrations enabling simultaneous determination of the association rate (kₒₙ; FIG. 1D) and equilibrium dissociation constant (K_{D}; FIG. 1E) for tens of thousands of target RNAs.

### Example 2

### RISC Association Proceeds via Binding of the Seed Region

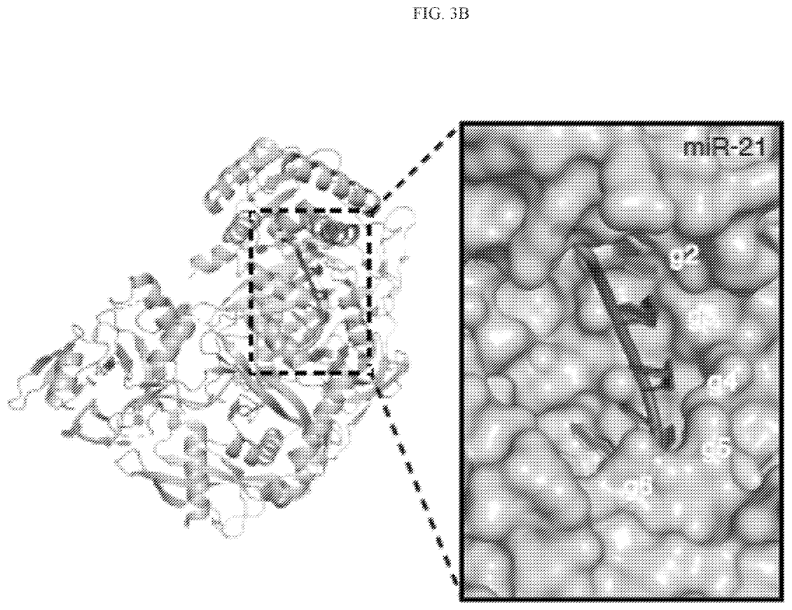

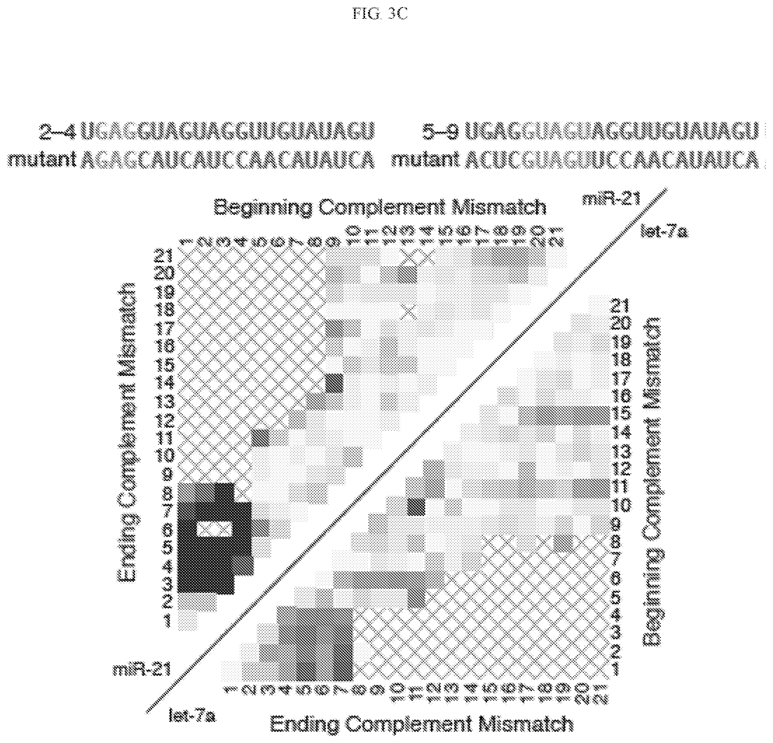

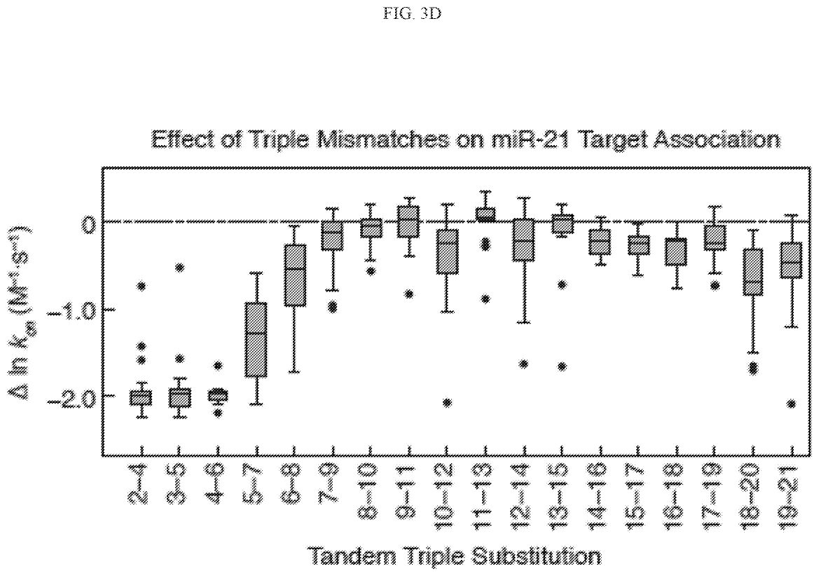

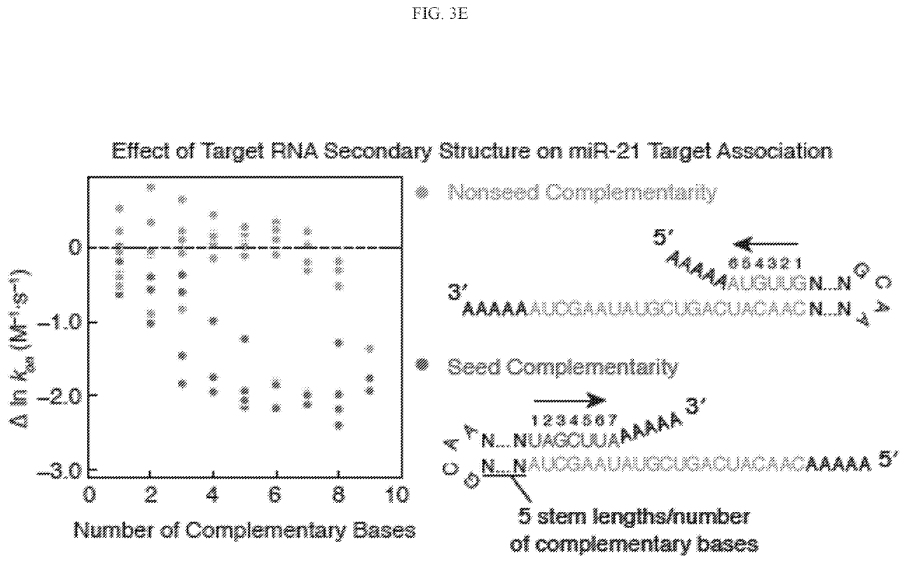

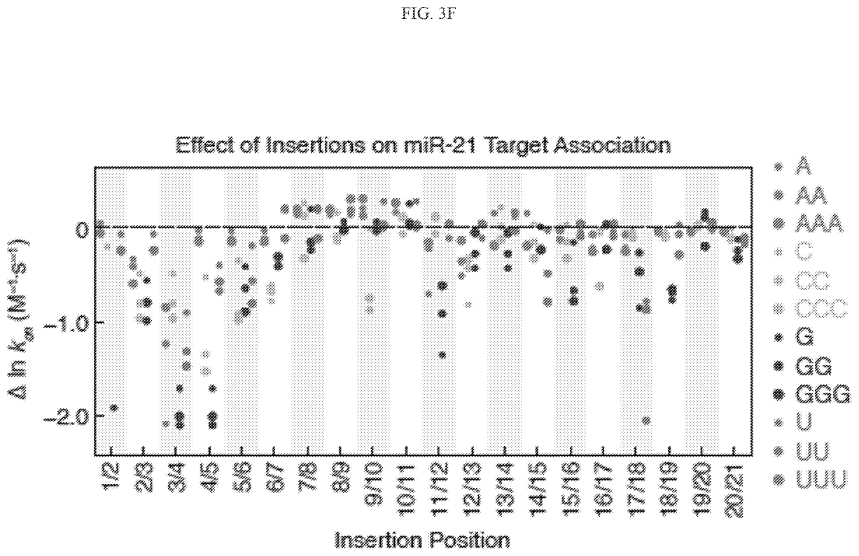

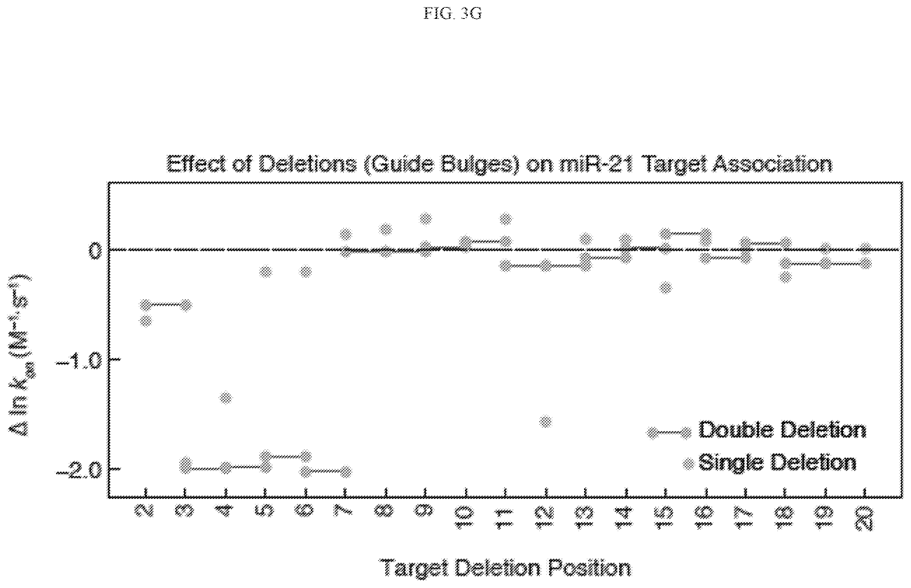

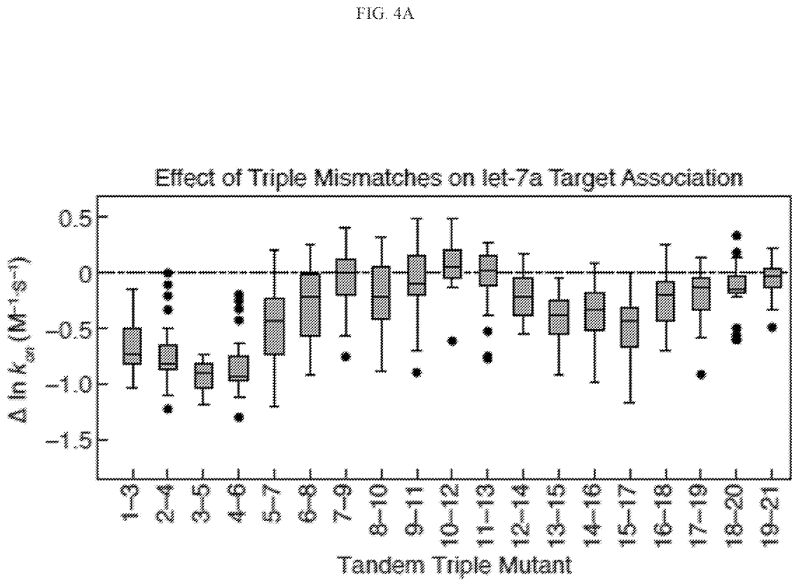

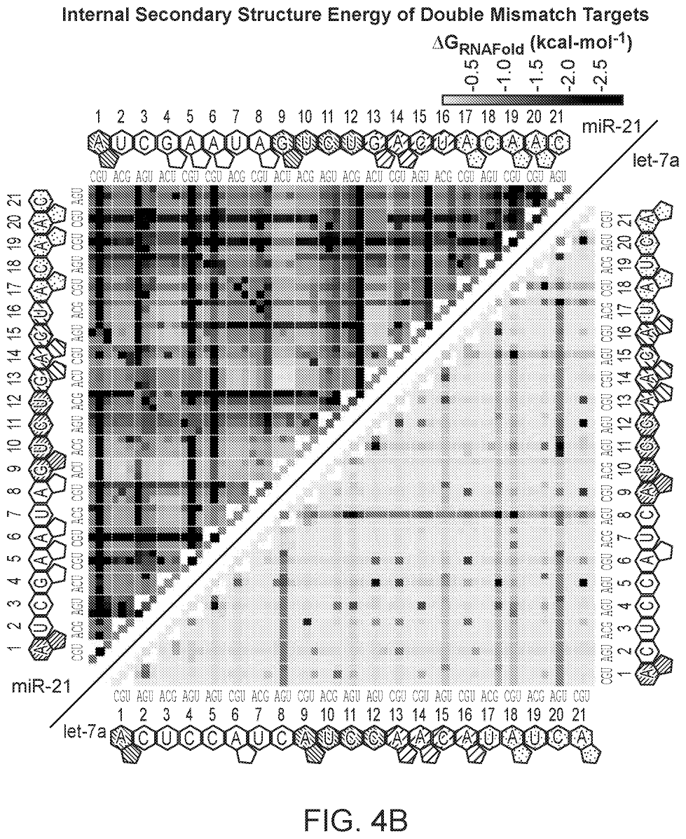

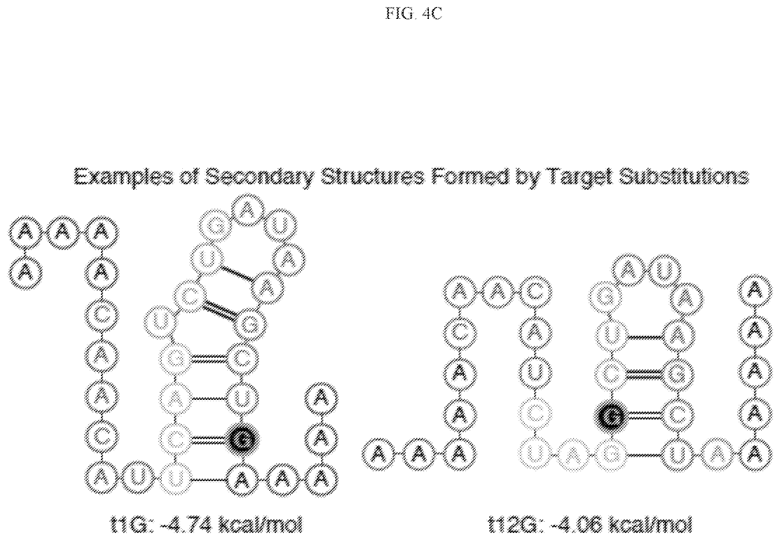

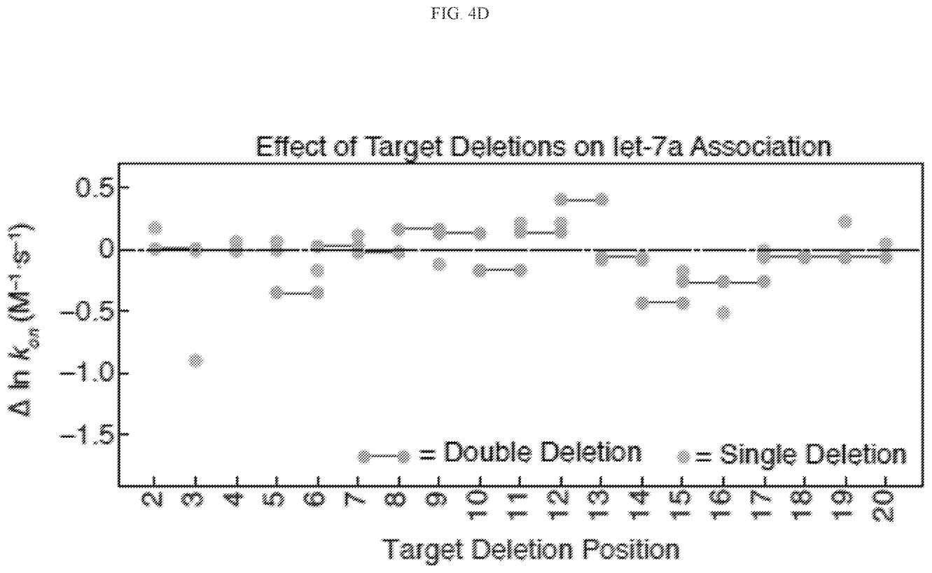

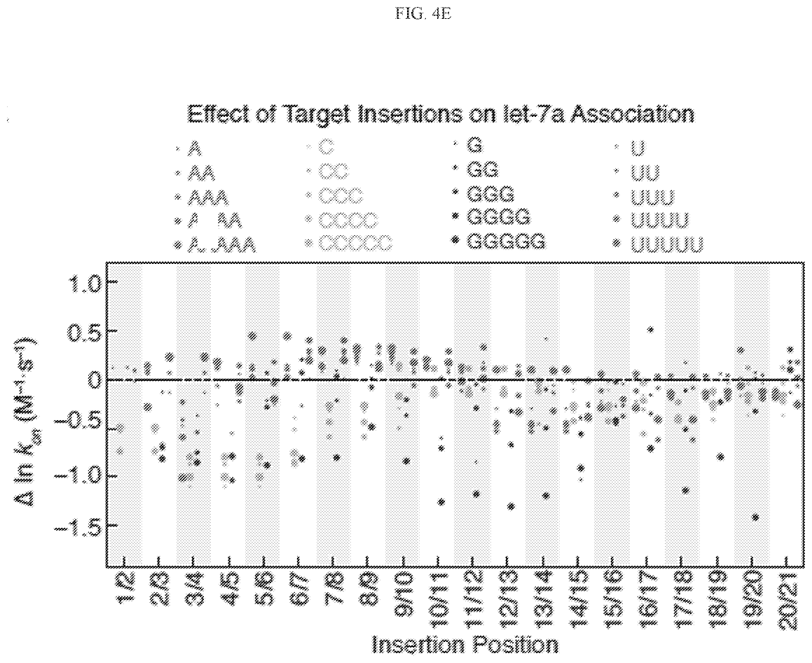

Argonaute facilitates target finding by accelerating the association rate to near diffusion limits (Wee et al., 2012; Salomon et al., 2015). Consistent with previous results, it was found that guide:target complementarity within the seed determines the rate at which RISC finds its target (FIGS. 3A-3D and FIG. 4A) (Wee et al., 2012; Salomon et al., 2015). For both miR-21 and let-7a, mismatches with seed nucleotides g2-g5 most slowed association of RISC with target RNA; these nucleotides are preorganized into a helical geometry and accessible for the initial target search (FIG. 3B) (Schirle et al., 2014; Salomon et al., 2015). Mismatches at target position t3 most impaired RISC association for both miR-21 (6.3-7.4 fold reduction for single mismatches) and let-7a (1.4-2.3 fold reduction for single mismatches; FIG. 3A). For both miRNA guides, the absence of complementarity to seed position g7 only modestly slowed RISC association, while mismatches at position g8 had no detectable effect on the RISC association rate.

Mismatches outside the seed generally slowed the association rate of RISC with target <2 fold (FIGS. 3A-3D and FIG. 4A). The target libraries for let-7a and miR-21 contained RNA with stretches of mismatches starting at every target position and ending at every other position: provided the target was fully complementary to the let-7a or miR-21 seed, target association rate was unaffected by even 13 contiguous mismatches (FIG. 3C). Among the 6,714 let-7a and 5,308 miR-21 targets containing at least a 6mer seed for which association was measured at three or more concentrations, non-seed mismatches primarily slowed RISC association by sequestering the target site in a stable secondary structure. RNAfold (Lorenz et al., 2011) was used to predict the structure of every target sequence in each library. Internal secondary structures more stable than −1.5 kcal·mol⁻¹ explained 67% (407 targets) and 63% (1,281 targets) of the >2 fold effects of mismatches outside of seed positions g2-g7 for let-7a and miR-21, respectively (FIG. 4B). For example, a target fully complementary to miR-21 but bearing cytosine (3.7 fold decrease) or guanine (3.1 fold decrease) instead of uracil at t12 slowed target finding as much as a single mismatch to the seed. Both of these target sequence changes stabilize a secondary structure that sequesters the seed-complementary region of the target in a hairpin (FIG. 4C). Another common cause of apparent secondary structure involves guanine substitutions at t1. The first base of a guide, g1, cannot pair with target base t1, because g1 is anchored in the phosphate-binding pocket of AGO2 (Ma et al., 2005; Parker et al., 2005; Frank et al., 2010). Nonetheless, a t1G slowed miR-21 RISC target finding ˜5 fold; t1G stabilizes an RNA hairpin (ΔG_{RNAfold, t}1G=−4.74 kcal·mol⁻¹) that occludes the target seed (FIG. 4C). Furthermore, the effects of double mismatches involving this position 1 substitution largely mirrored the stability of the predicted internal structure caused by this mismatch (FIG. 2A and FIG. 4B). For let-7a, the target sequences generally were predicted to form less internal structure. The library also included targets with progressively longer hairpins at each end of the target sequence, enabling systematic investigation of secondary structures effects at different positions. In contrast to seed occluding structures, secondary structures that sequestered non-seed pairing regions of the target had no effect on RISC association (FIG. 3E). These secondary structure tests reveal that target occlusion via RNA structure can significantly modulate RISC association kinetics, but only when the structure involves the seed sequence.

### Example 3

### Insertions and Deletions Minimally Affect RISC Association Rates

The effects of target insertions on RISC association have not been systematically studied. Except for insertion or deletions in the seed-pairing region, association rate of RISC to targets was largely unaffected by 1-7 nt insertions or 1-2 nt deletions in the target sequence (FIG. 3F and FIG. 4E). Insertions between positions 3 and 4 had the largest effects (up to 8 fold) on RISC association rates, likely because they preclude formation of a three- or four-nucleotide helix capable of nucleating binding (Schirle et al., 2014). Deletion of target nucleotides is predicted to require a bulge in the guide strand to accommodate flanking base pairs. For targets fully complementary to let-7a, only deletion of nucleotide t3 slowed target finding by more than 2 fold (FIG. 4D). In contrast, deletion of target nucleotide t3, t4, or t12 from fully complementary miR-21 targets, all reduced target association by >2 fold (FIG. 3G). Removing two consecutive nucleotides from fully complementary targets decreased miR-21 RISC association rate for deletions within the seed (>6 fold reduction), but deletions outside the seed-pairing target region did not alter RISC association kinetics (FIG. 3G). In general, insertions were better tolerated than deletions at the same target position. For example, for miR-21, inserting three adenosine, cytosine, or uridine nucleotides between t3 and t4 yielded faster association rates than deleting nucleotide t3.

Together, these data suggest that target bulges in the seed-pairing region, which are predicted to face the solvent, are more readily accommodated than unpaired seed nucleotides in the guide, which face the protein.

### Example 4

### Seed Complementarity is the Most Important Determinant of Binding Affinity, but Additional 3′ Supplemental Pairing is Needed for High-Affinity Binding by Some miRNAs

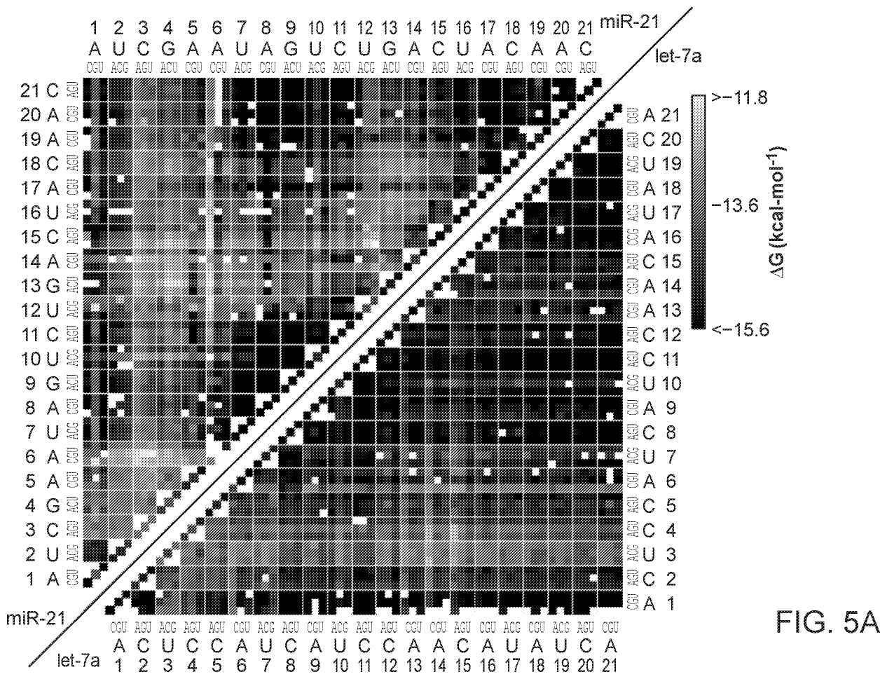

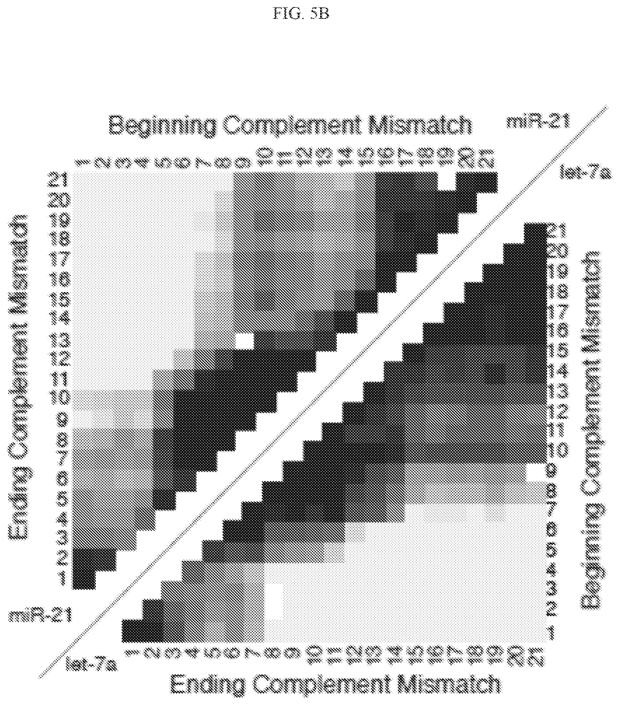

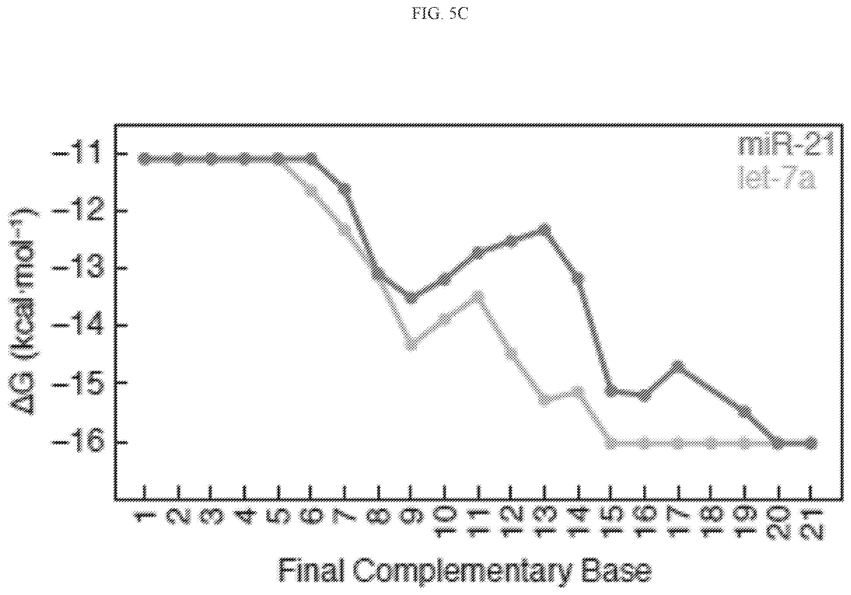

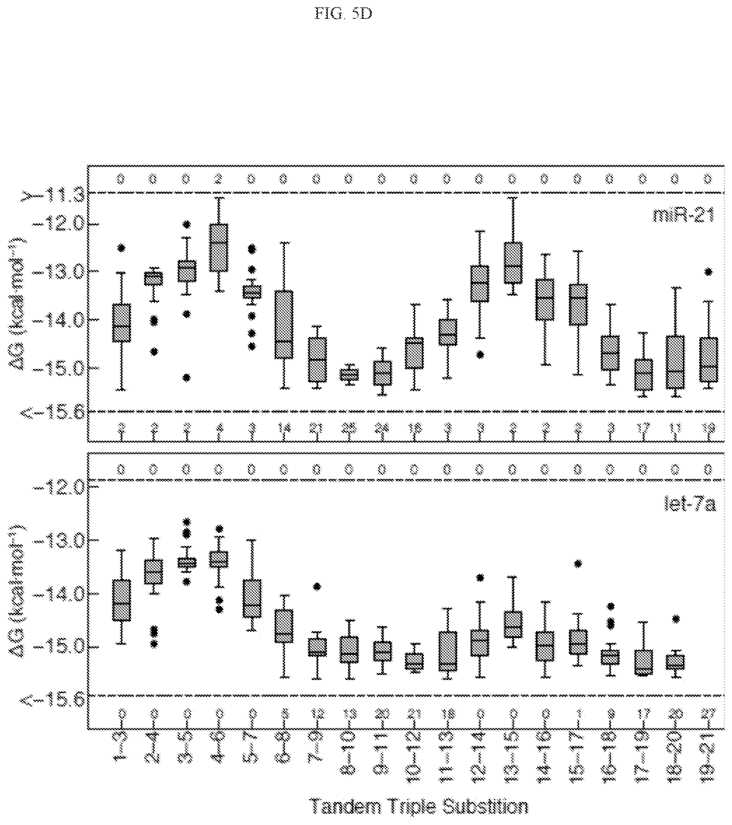

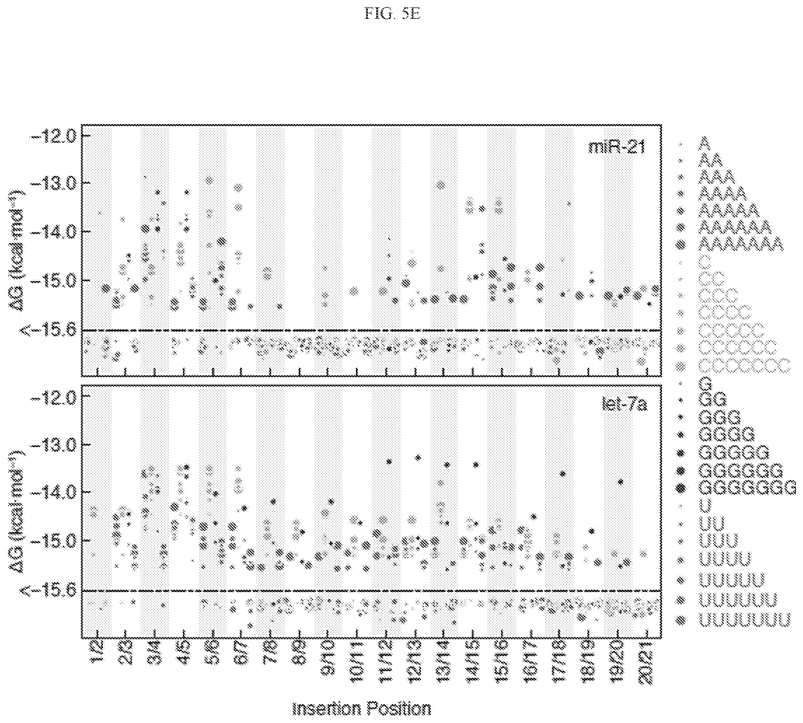

Repression of mRNA expression by miRNAs requires that RISC remain bound to an mRNA long enough to recruit deadenylases and other nucleases that degrade the target mRNA. To identify which target bases determine how long RISC remains bound to its target RNA, RISC equilibrium binding affinity to mismatched targets was measured. For let-7a, mismatches at positions 3 and 4 led to the largest changes in binding affinity. In contrast, for miR-21, mismatches throughout the seed or in the 3′ supplemental region had surprisingly similar effects on RISC binding affinity (FIGS. 5A and 5D). When comparing the overall affinities for each RISC, it was found that let-7a targets containing mismatches at the same positions as miR-21 targets were often bound with higher affinity (FIGS. 5A, 5B, and 5D). Both the reliance of miR-21 on supplemental pairing and the overall lower affinities of miR-21 RISC for its targets are likely consequences of the substantially lower GC content in the miR-21 seed sequence.

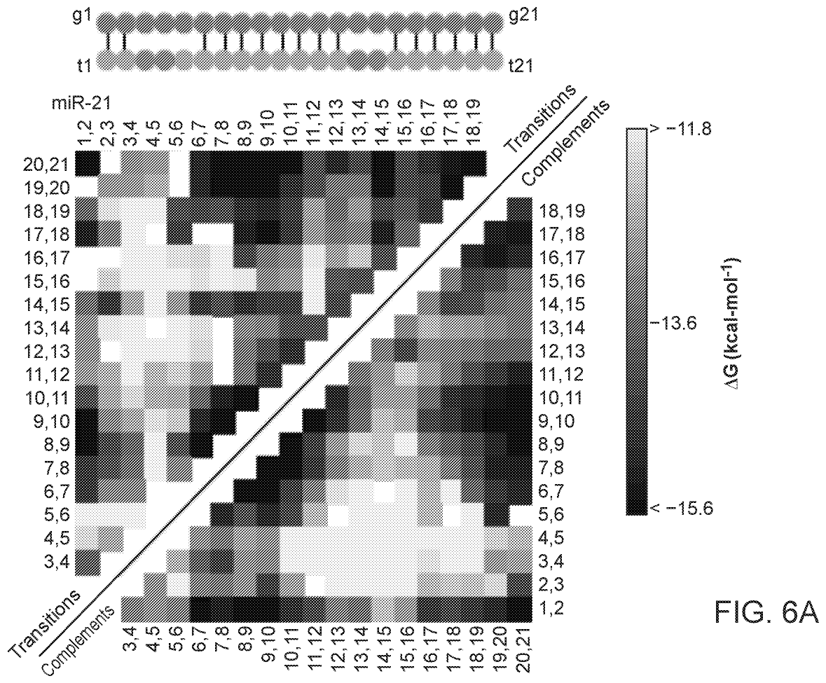

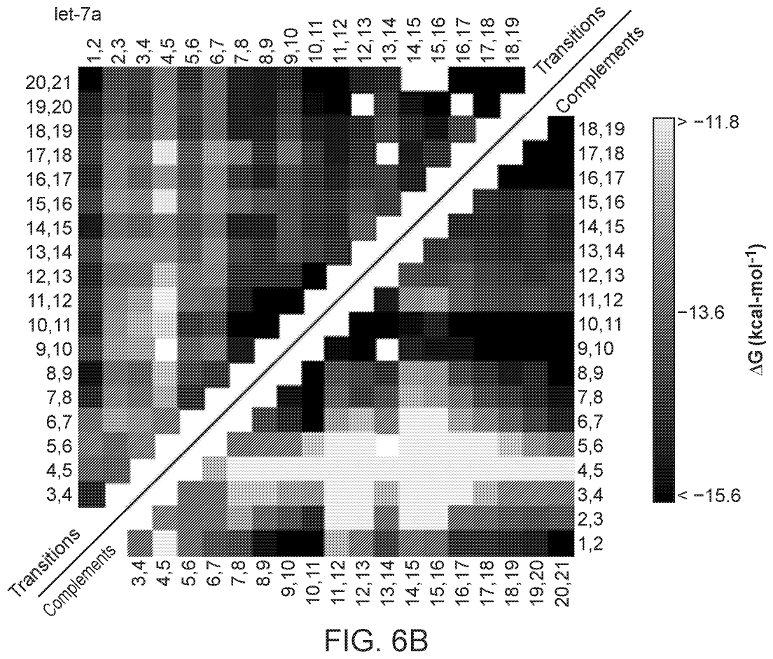

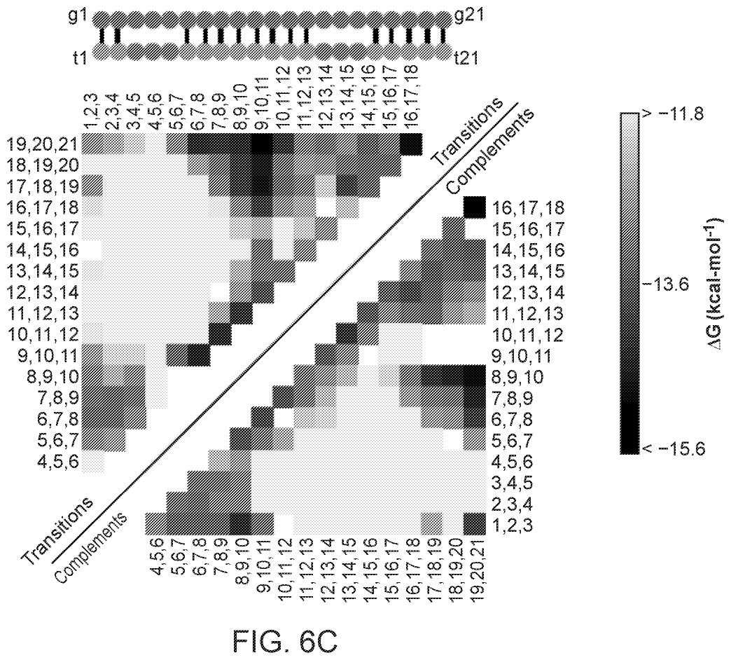

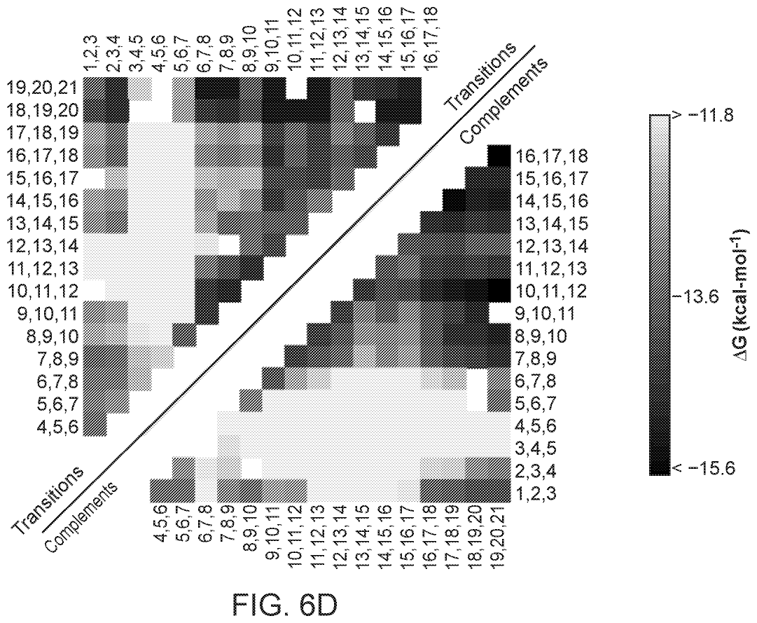

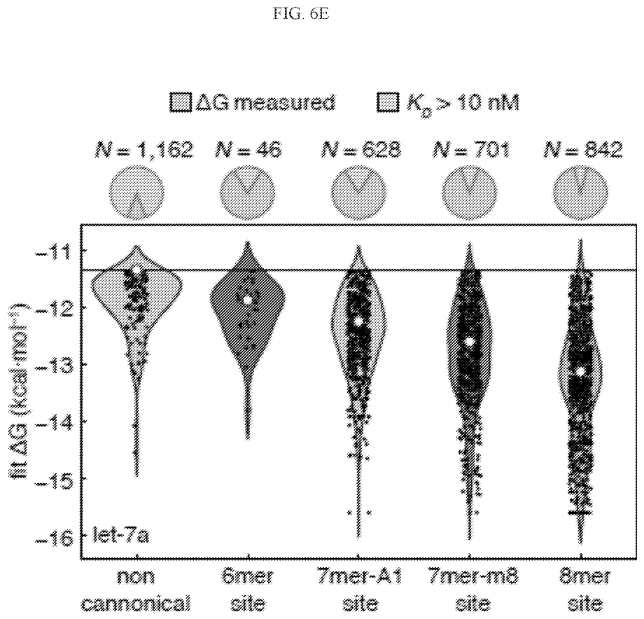

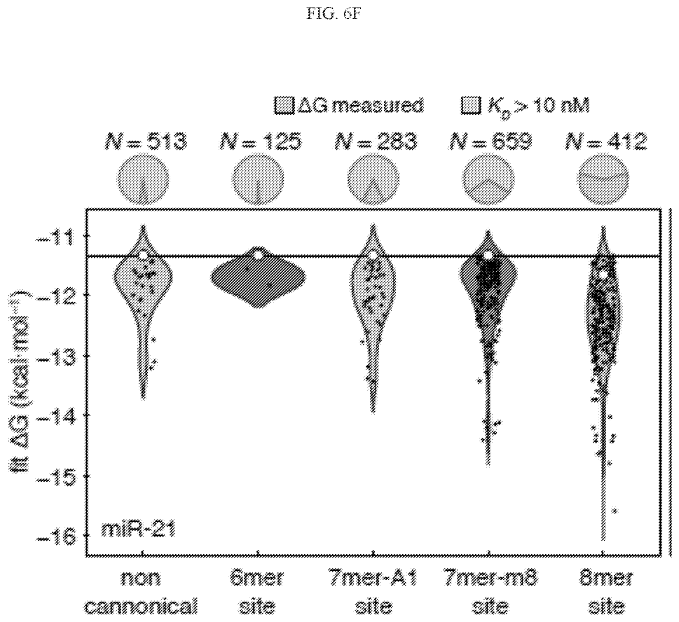

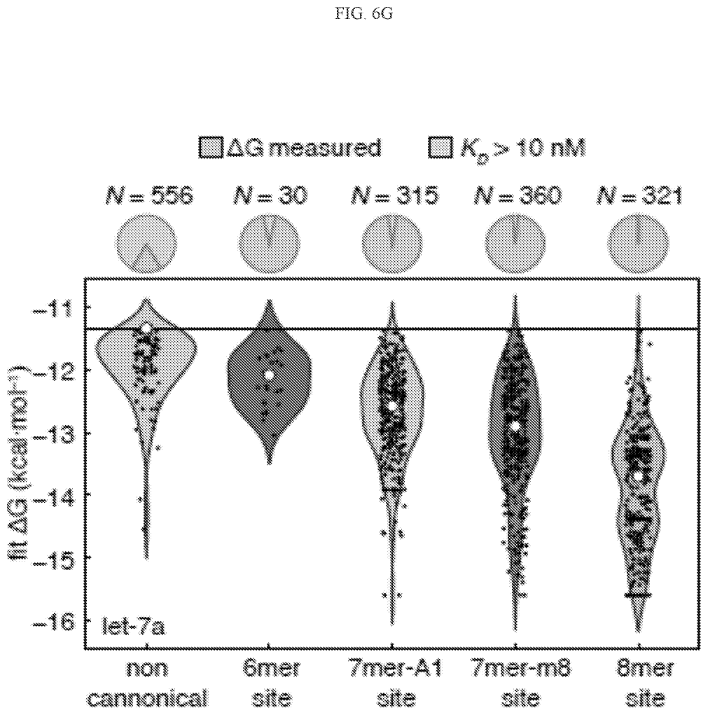

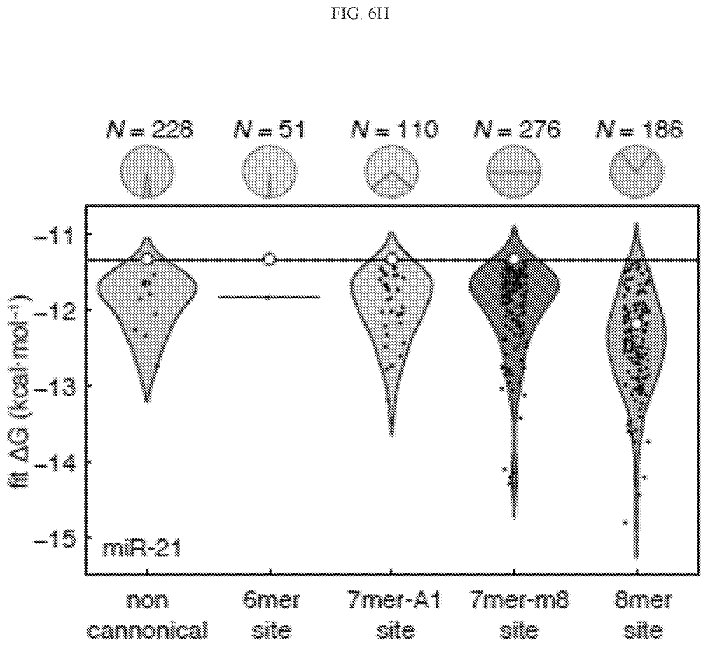

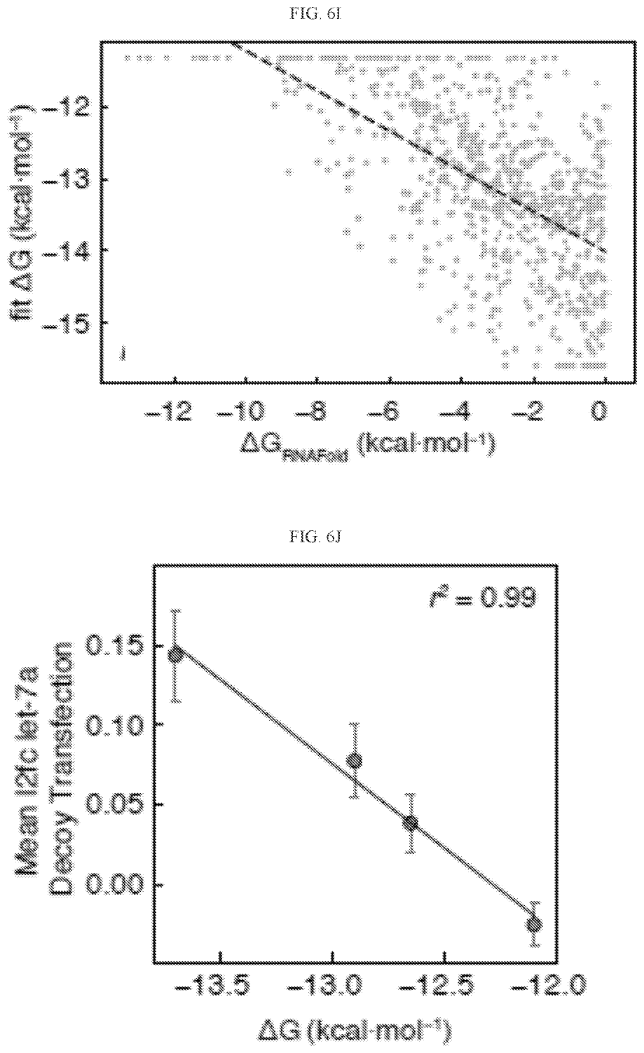

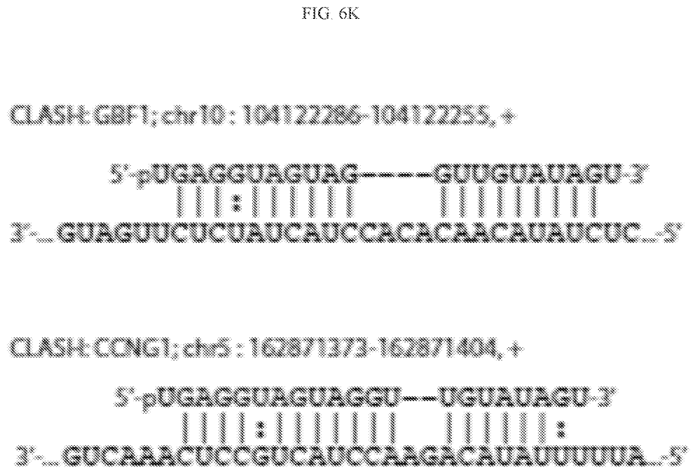

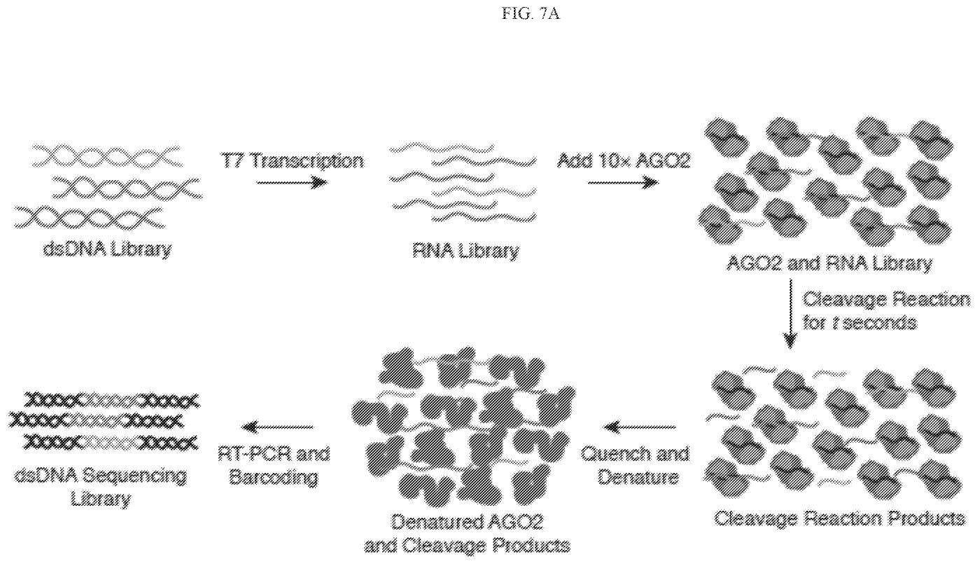

Computational algorithms designed to predict miRNA target sites differ widely in their ability to capture let-7a or miR-21 RISC affinity for target sequences. For both let-7a and miR-21, 8mer sites (targets containing complementarity at positions 2-8 and an A at position 1) bound with the highest affinity on average, followed by 7mer-m8 sites (targets containing complementarity at positions 2-8), 7mer-A1 sites (targets containing complementarity at positions 2-7 and an A at position 1), and 6- mer sites (targets containing complementarity at positions 2-7; FIGS. 6E and 6F). A range of affinities to predicted targets containing the same seed types (e.g., 8mer, 7mer-A1) was observed. Modeling secondary structures showed that some of this variance could be explained by the formation of internal structure in the target sequence (R²=0.38 for 8mer seed targets; FIG. 6I). To account for this, targets predicted to have more stable internal structures (ΔG_{RNAfold}<−2 kcal·mol⁻¹) were removed and the distribution of affinities for each site type (FIGS. 6G and 6H) was replotted. This threshold was selected to remove targets containing stable structures that measurably reduce binding affinity (FIG. 6I). Comparing the site types, it was found that an A at position 1, which binds in the AGO2 t1A binding pocket, increased the binding affinity by an average of 1 kcal·mol⁻¹ for both guides (8mer vs 7mer-m8 site), which likely explains the difference in target efficiency for these site types. This effect can be explained by sequence-specific contacts between AGO and the t1 nucleotide of the target. It has been previously shown that the phosphate-binding pocket of human AGO2 recognizes an unpaired adenosine via water-mediated amino acid hydrogen bonds (Schirle et al., 2014). The affinities measured for the same site types differed significantly between let-7a and miR-21, with all miR-21 site types binding with lower affinity than the corresponding site type for let-7a (FIGS. 6G and 6I). This difference in overall affinity is again likely a result of the low GC content seed for miR-21, and may suggest that 3′ supplemental pairing is more important for miR-21 and similar miRNAs.

To test if these differences in binding affinity reflect differences in regulation of mRNA targets, the median binding affinities measured for each seed type were compared to previously published RNA-seq data collected following transfection of a let-7a decoy into 3T3 cells (Werfel et al., 2017). Considering only RNAs containing a single canonical binding site in their 3′ UTR, RNAs by seed type were binned and a strong correlation (R²=0.99) between binding affinity and the mean loge change in target abundance for each seed type (FIG. 6J) was found, suggesting that binding affinity is a key factor in regulation by microRNAs.

### Example 5

### Central Pairing can Reduce RISC Binding Affinity

Guide pairing to target bases t9 and t10 reduces the binding affinity of RISC to its targets, leading to the proposal that pairing at these central positions requires an unfavorable conformational change (Schirle et al., 2014; Salomon et al., 2015). The binding affinity of targets containing central mismatches with varying degrees of additional complementarity was explored. For both miR-21 and let-7a, it was found that Watson-Crick pairing of central base pairs (9-12) contributed less to binding affinity than the seed and 3′ supplemental regions: most variants with 1-3 mismatches in the central region bound with higher affinity than the detection threshold of 10 pM (FIGS. 5A and 5B). Given the relative insensitivity of binding kinetics to most single, double, and triple mismatches in the central region, the contributions of central bases to binding were examined in greater detail by looking at all stretches of contiguous mismatches (FIGS. 5B and 5C). In these variants, it was observed that extensive base pairing in the central region reduces binding affinity: RISC affinities for miR-21 targets with continuous mismatches starting at base t10 and proceeding to different endpoints were higher than for miR-21 targets with continuous mismatches starting at base t13 and extending to the same positions. Indeed targets containing complementary bases through position t12 had consistently lower affinity than those containing complementary bases through position 11, which in turn were lower than targets only containing complementary bases through position 10 (FIGS. 5B and 5 C). This same ordering of affinities (ΔGₜ₂₋ₜ₁₁>ΔGₜ₂₋ₜ₁₀>ΔGₜ₂₋ₜ₉) was observed for let-7a, but the affinity decreased with additional binding through base t11 rather than through base t13 (FIG. 5C).

This observation indicates that for most biological targets, which have little or no 3′ pairing, complementarity at positions 10-12 can reduce binding affinity, and this counterintuitive effect should be accounted for when predicting miRNA targets. When looking at the effects of central bases on the binding affinity of targets containing little or no seed complementarity, central pairing generally increased binding affinity (FIG. 5B and FIGS. 6A-6D), suggesting that the conformation of centrally paired targets may specifically destabilize seed binding. However, for miR-21 RISC, targets with complementarity at t10-t21 were lower affinity than targets with complementarity from t11-t21, providing evidence that a destabilizing conformational change still impacts the affinity of these targets.

### Example 6

### Ago can Tolerate Large Target Insertions without Substantial Decreases in Binding Affinity

Bypassing central pairing (g9-g12) via mismatches may prevent an energetically unfavorable conformational change (Wee et al., 2012). In fact, even large central bulges in RISC targets had little effect on binding affinity (FIG. 5E). Each target library was designed to contain all 560 possible targets bearing 1-7 nucleotide insertions at every nucleotide position (452 measured for miR 21; 463 measured for let-7a). For both let-7a and miR-21, only 258 let-7a and 156 miR-21 targets detectably reduced RISC binding affinity. Most (53% for miR-21 and 45% for let-7a) of these corresponded to insertions in the seed. For miR-21, only 9 of 95 (17 unmeasured) target insertions between t8 and t12 detectably reduced binding affinity. These data suggest that RISC first finds and binds to the seed-complementary region of a target, then loops out intervening, non-complementary target sequences in order to pair with complementary 3′ supplementary target bases. Large bulges such as this have been described previously for miR-122, which binds the Hepatitis C viral RNA at a site predicted to contain a large central hairpin (Luna et al., 2015). The data suggest that this binding mode may be more common than previously appreciated: four target bases complementary to the 3′ supplementary region are predicted to occur by chance every 256 nucleotides.

### Example 7

### Mechanisms for High-Affinity Binding to Noncanonical Targets

Although the seed sequence is the primary specificity determinant for target binding, miRNA sequences outside the seed are also evolutionarily conserved. Thus, targets with incomplete seed complementarity but extensive non-seed complementarity likely contribute to conservation of miRNA sequences. Furthermore, several classes of noncanonical targets have been proposed to support RISC binding, including nucleation bulges and centered sites (Shin et al., 2010; Chi et al., 2012). However, predicting binding to noncanonical targets remains difficult, and many prediction algorithms do not attempt to identify noncanonical targets.

The target library included 513 miR-21 and 1,162 let-7a noncanonical targets predicted by different algorithms or identified by CLASH. These putative noncanonical targets include 3′-compensatory and centered sites, as well as sites containing a single G:U wobble in a 6mer seed (Betel et al., 2010). The majority (95% for miR-21 and 89% for let-7a) of these noncanonical targets had no observable binding at the concentrations measured (FIGS. 6E and 6F). The two highest affinity let-7a noncanonical targets formed G:U wobble pairs with the let-7a seed that were bolstered by 3′ supplemental pairing (FIG. 6K). After removing targets whose binding sites are predicted to be sequestered in a stable structure (ΔG_{RNAfold}<−2 kcal·mol⁻¹), only 18.7% (104) of the 556 remaining let-7a and 5% (11) of the 228 remaining miR-21 putative noncanonical sites were bound with a dissociation constant less than 10 nM (ΔG<−11.3 kcal·mol⁻¹; FIGS. 6G and 6H). Thus, most noncanonical targets identified by prediction algorithms or CLASH correspond to low affinity binding sites unlikely to be substantially occupied in vivo (Agarwal et al., 2015).

The libraries included many nucleation bulge sites, sequences in which a nucleotide inserted between t5 and t6 can base pair with g6. The best studied let-7a nucleation bulge site, UAACCUC (Chi et al., 2012), occurred in 32 biological targets in the let-7a library. let 7a RISC bound these targets only weakly: the median affinity was 9.04 nM (ΔG=−11.4 kcal·mol⁻¹), and binding to 15 sites was below the limit of detection (K_{D}>10 nM). Just three sites, which all included 5-7 additional non-seed base pairs, bound with an affinity <2 nM (ΔG<−12.3 kcal·mol⁻¹). The highest affinity site (ΔG=—13 kcal·mol⁻¹), which bound RISC with affinity similar to a 7mer-m8 site, contained contiguous base pairing from t2-t13 with an A bulge between t5 and t6 and a G:U wobble pair at t11. The second highest affinity site (ΔG =−12.8 kcal·mol⁻¹) included contiguous base pairing from t2-t15 interrupted by an A bulge between t5 and t6, a mismatch at t10, and a G:U wobble pair at t15. The third site contained contiguous base pairing from t2-t9 interrupted by an A bulge between t5 and t6, a large central target bulge, and contiguous pairing from g12-g17 with G:U wobble pairs at t12, t15, and t16 (ΔG=−12.5 kcal·mol⁻¹). Thus, only UAACCUC sites buttressed by non-seed pairing bind RISC with high affinity; the majority of such noncanonical sites bind with low-affinity, and, thus, are unlikely to function in vivo.

In addition to nucleation bulges, the library included targets with different extents of complementarity, but lacking a canonical seed match. These sites included targets similar to centered sites, which contain 11-12 bases of contiguous central complementarity (Shin et al., 2010). The binding affinities of these targets demonstrated that the length of contiguous complementarity needed for high-affinity binding depends on both guide sequence and position within the guide. For example, let-7a RISC bound targets with uninterrupted complementarity from t5-t17 (ΔG=−12.2 kcal·mol⁻¹) and t5-t16 (ΔG=−11.9 kcal·mol⁻¹ and −12.0 kcal·mol⁻¹ for two sequence variants), with affinities similar to a 6mer seed match. In contrast, targets complementary to let-7a from t5-t15 bound with affinity (ΔG>−11.3 kcal·mol−1) lower than a 6mer seed match. For miR 21, but not let-7a, targets containing contiguous complementarity from t11-t21 bound more tightly than either 7mer-m8 or 7mer-t1A sites (FIG. 5B). The data suggest that centered sites or extensively complementary 3′ only sites could potentially be functional, but the length and position of complementarity required likely depends on the distribution of GC content, particularly within the seed, for an individual miRNA.

Imperfect seed complementarity has been proposed to render RISC binding dependent on 3′ compensatory pairing (Bartel, 2009). Supporting this view, many targets both imperfectly matching the seed and bearing an additional two mismatches outside the seed failed to detectably bind RISC binding (FIGS. 6A and 6B). For example, let-7a 3′ compensatory targets bearing both two target nucleotide transversions at positions t4 and t5 and two additional, adjacent target nucleotide transversions at any position between t7 and t21 failed to detectably bind (K_{D}>10 nM). For other seed positions this effect was restricted to specific 3′ positions: combining a t2t3 transversion with four of the five possible dinucleotide transversions between t11 and t16 eliminated detectable binding (K_{D}>10 nM) for let-7a RISC, but dinucleotide transversions between t16 and t21 were tolerated. For many of these targets, central base pairing (t9-t12) enhanced binding affinity. Paradoxically, central base pairing (t9-t12), which destabilizes binding of seed-matched targets, was required for seed-mismatched target binding. Similar trends were observed when three consecutive seed mismatches were combined with three consecutive mismatches in the central or 3′ region, which typically abolished observable binding (FIGS. 6C and 6D). It is concluded that target sites without complete seed complementarity bind only when they contain extensive complementarity distal to the seed: two non-seed mismatches that would have little effect on binding for seed-matched sites can disrupt RISC binding in the context of seed mismatches.

### Example 8

### High-Throughput Measurement of RISC Cleavage Rate

siRNAs are typically designed to be fully complementary to their target. This design paradigm has been challenged by evidence that AGO cleavage activity can be enhanced by specific guide:target mismatches (Tang et al., 2003; Haley and Zamore, 2004; Ameres et al., 2007). Moreover, mismatches can allow siRNAs to discriminate between targets that differ by a single nucleotide (Dykxhoorn et al., 2006; Schwarz et al., 2006; Pfister et al., 2009). However, identifying mismatches that improve siRNA efficacy or specificity currently requires testing large numbers of individual siRNAs.

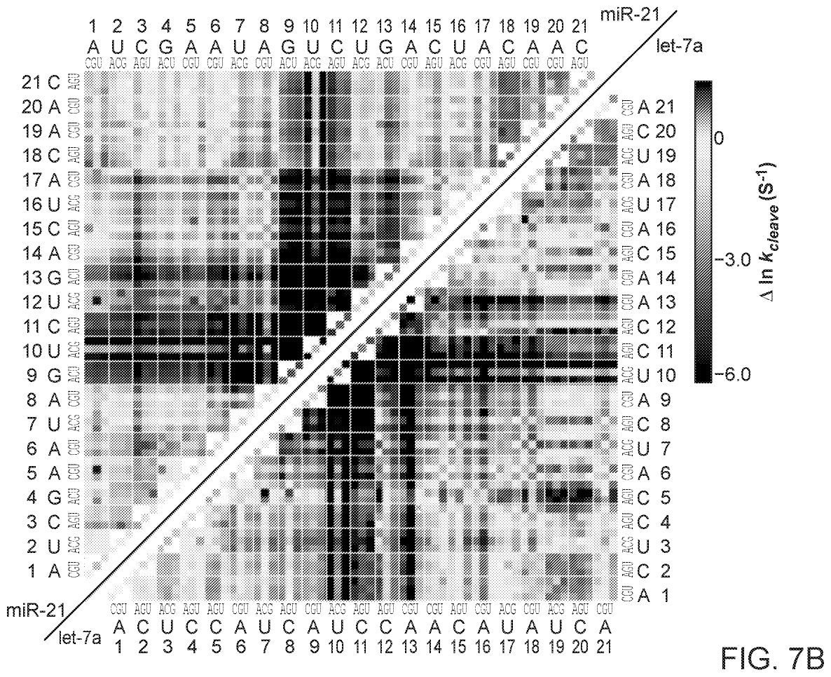

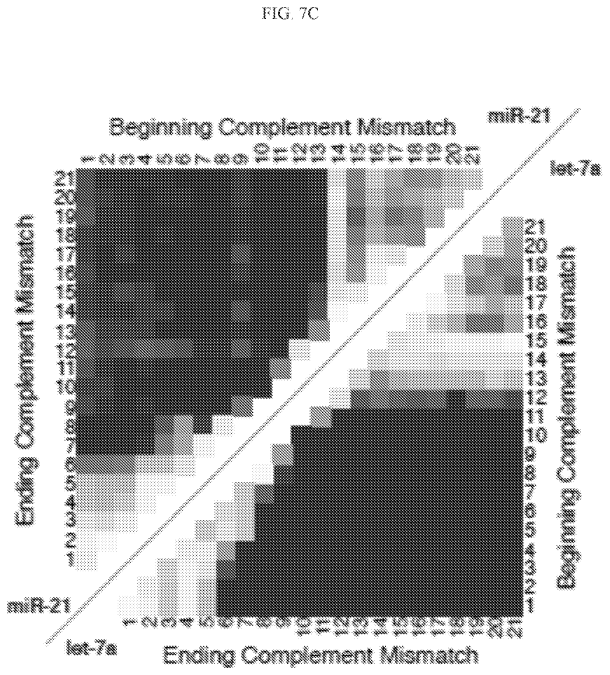

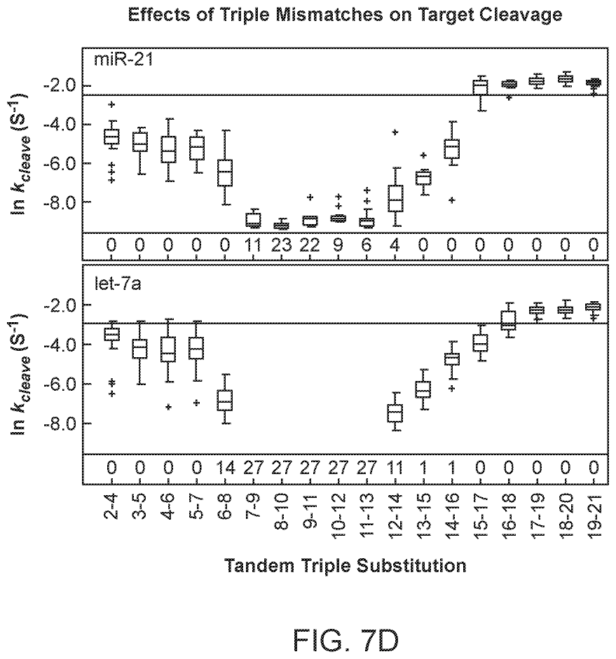

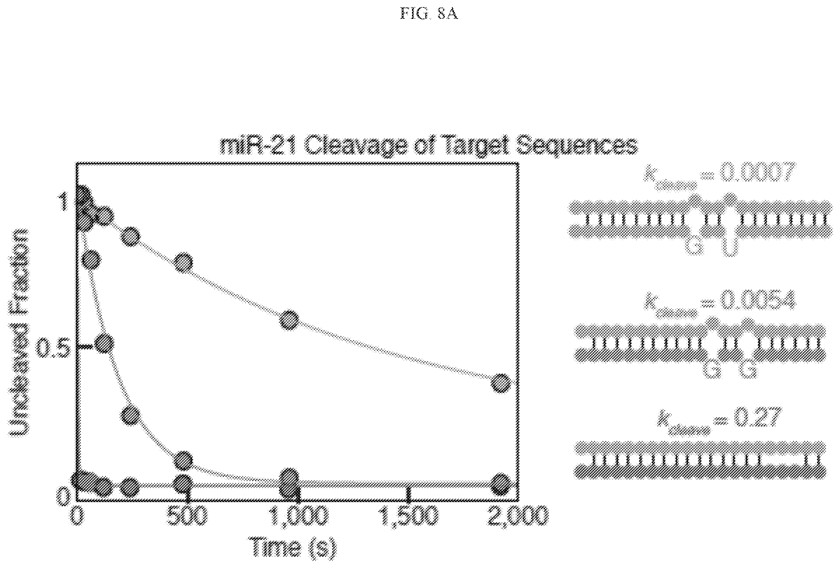

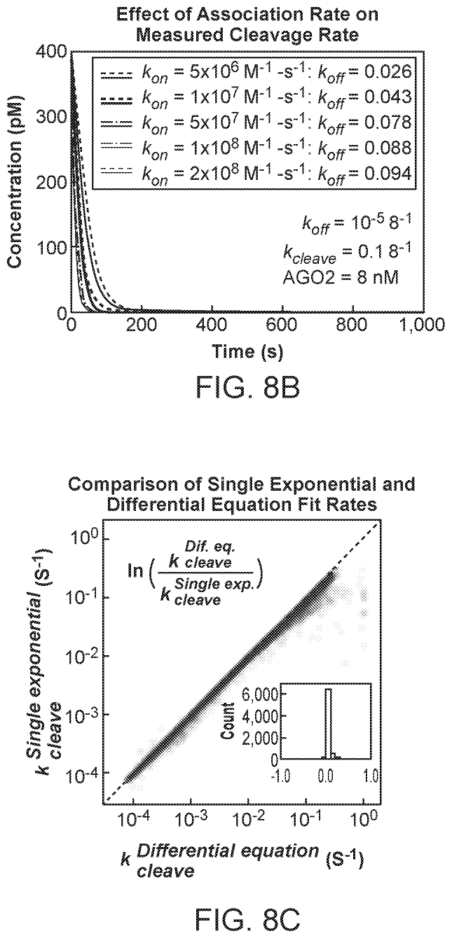

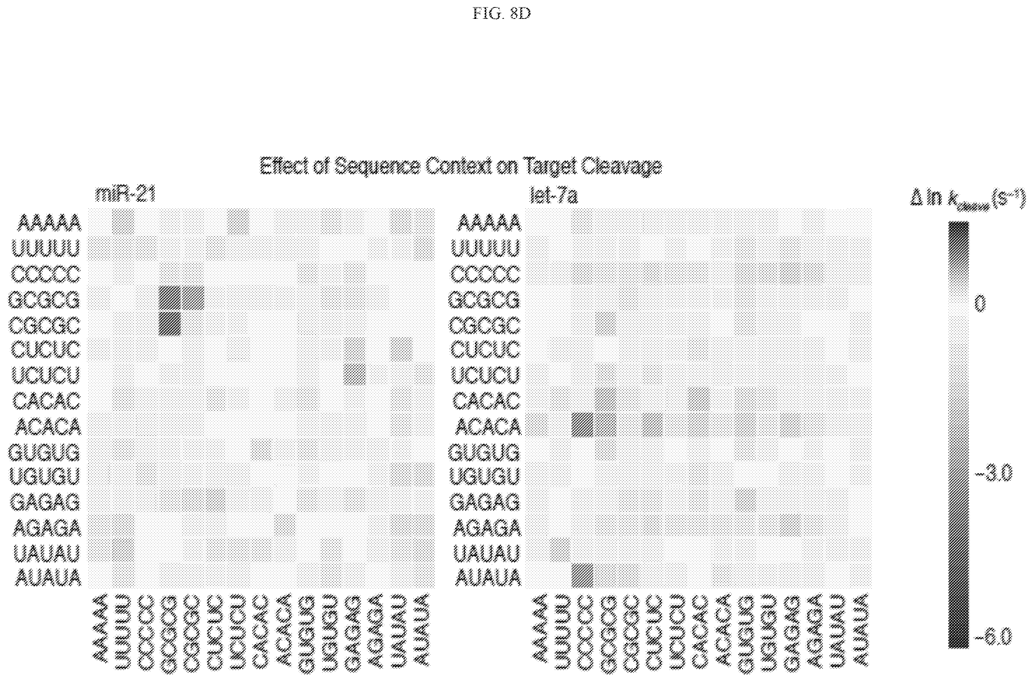

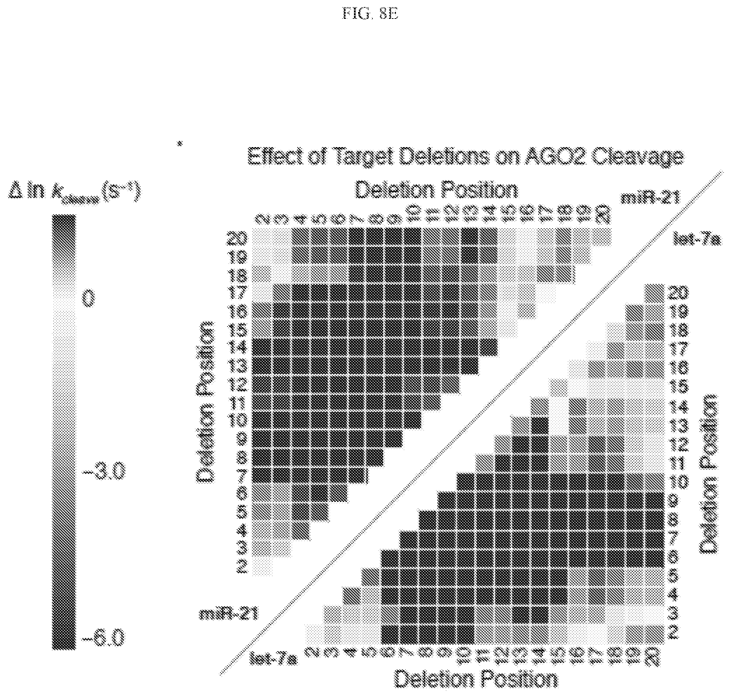

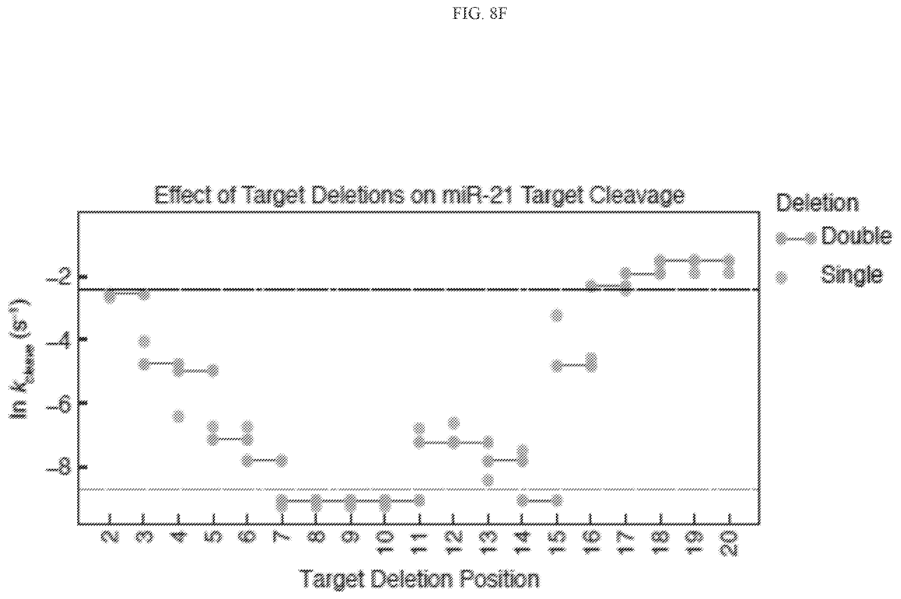

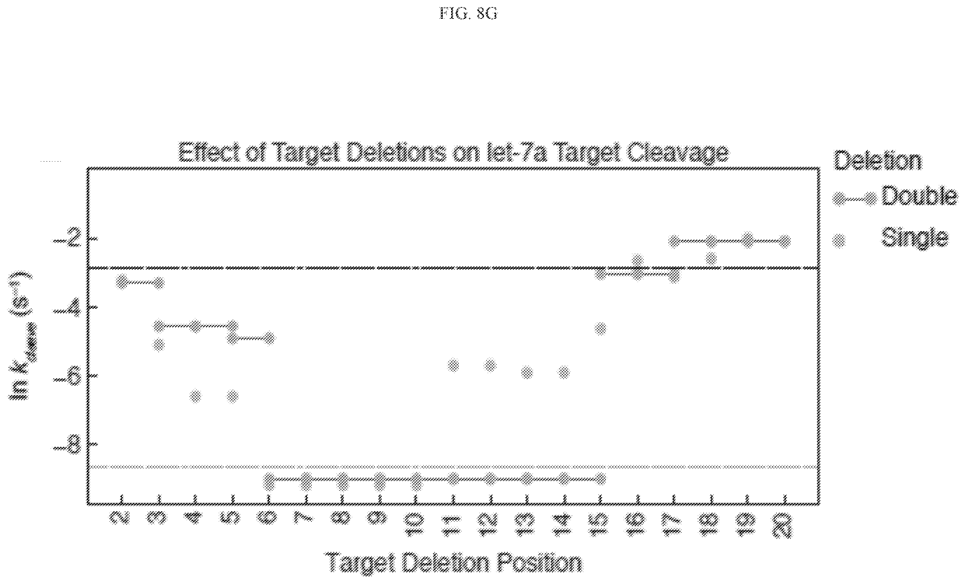

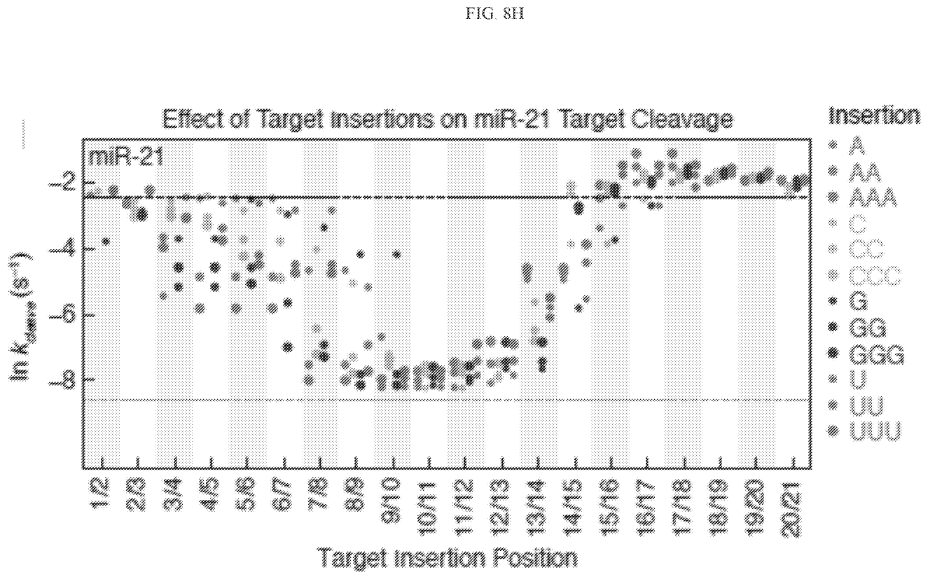

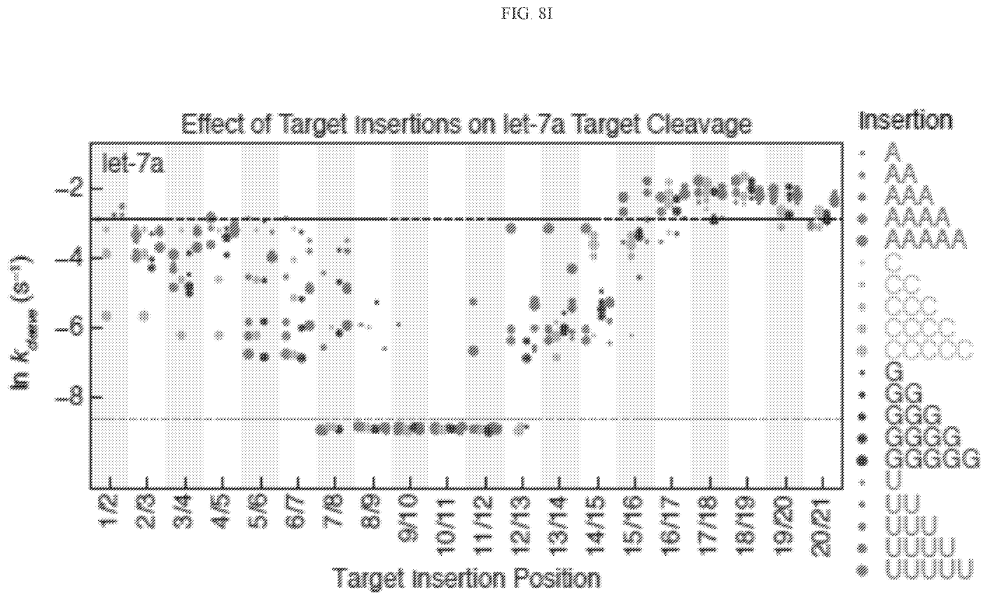

RISC Cleave-'n-Seq (RISC-CNS) was developed to enable high-throughput measurements of RISC cleavage rates, rapidly identifying favorable guide:target mismatches for an individual siRNA sequence (FIG. 7A and FIGS. 2A and 2B). RISC-CNS begins by transcribing a DNA library (in this case the same libraries designed for array experiments), then incubating the RNAs with a 10 fold molar excess of RISC to achieve single-turnover conditions. Cleavage is measured after various times by reverse transcribing and sequencing the targets remaining uncut. Normalized sequencing data was fit to a single exponential curve to determine the cleavage rate for each of the 22,607 let-7a and 7,841 miR-21 variants (FIG. 8A). In theory, cleavage rates for variants with slow association rates (e.g., seed mismatches) or exceptionally fast cleavage rates may deviate from the single exponential approximation (FIG. 8B). In practice, the exponential approximation yields essentially the same values as a model incorporating the measured relative association and dissociation rate (FIG. 8C), so the values from the simpler, single-exponential, model were used. Like the array binding experiments, five nucleotides of RNA sequence flanking each side of the target site are accessible in RISC-CNS, allowing measurement of the effect of 225 different five-nucleotide flanking contexts on a target fully complementary to let-7a or miR 21 bound to AGO2. Flanking sequences had only modest effects on cleavage rate (k_{cleave}, mean±S.D. of 0.077±0.024 s⁻¹ for miR 21 and 0.037±0.013 s⁻¹ for let 7a), suggesting that the rates measured by RISC-CNS are generally insensitive to local secondary structure or biases from PCR amplification or high-throughput sequencing (FIG. 8D).

### Example 9

### Some, But Not All, Target Mismatches Flanking the Cleavage Site Inhibit Cleavage

RISC cleaves its RNA target at the phosphodiester bond linking target nucleotides t10 and t11 (Elbashir et al 2001), and central base pairing (g9-g12) between the guide and target is required for efficient target cleavage (Haley and Zamore, 2004; Ameres et al., 2007; Wee et al., 2012) as it moves the scissile phosphate into the catalytic site (Ma et al., 2005; Parker et al., 2005). RISC-CNS revealed that for otherwise fully complementary targets, mismatches at t10 and t11 caused the greatest reduction in target cleavage rate; cleavage was not detectable for many targets containing these mismatches (FIG. 7B). For both let-7a and miR 21, all g10g11:t10t11 double mismatches caused at least a 270 fold decrease in cleavage rates, with seven out of nine and nine out of nine double mismatches exhibiting cleavage rates below our detection threshold for let-7a and miR 21, respectively. In contrast, the three g9:t9 mismatches reduced the rate of target cleavage by miR 21 RISC an average of 38 fold, but had a much smaller average effect (9 fold) on let-7a RISC (FIG. 7B). For all possible let-7a triple mismatches at t9-t11 and 21 of 27 miR 21 triple mismatches at t9-t11, cleavage was undetectable, corresponding to a >500 fold decrease in cleavage rate (FIG. 7D). For both let-7a and miR-21, a mismatch produced by changing t10U to t10C was better tolerated than other base substitutions, likely because substitution of another pyrimidine at the cleavage site is less disruptive to the helical geometry required for cleavage (FIG. 7B). When a t10U-to-t10C mismatch was combined with a 3′ mismatch between t18 and t21, the cleavage rate was typically faster than the 10C substitution alone (11 of 12 variants for let 7a and 12 of 12 variants for miR 21). These 3′ mismatches actually rescued the effect of a 10C mismatch so that 6 of 12 had a cleavage rate within 3 fold of a perfect complement target for both let 7a and miR 21 (FIG. 7B). Moreover, mismatches at t13, a position not usually considered part of the central region often perturbed cleavage. For example, the cleavage rates of targets containing t13 mismatches (t12C=0.002 s⁻¹, t12G<0.0002 s⁻¹, and t12U=0.011 s⁻¹ for let 7a and t13A =0.021 s⁻¹, t13C=0.004 s⁻¹, and t13U=0.005 s⁻¹ for miR 21) were typically slower than the cleavage rates of targets containing t12 mismatches (t12A=0.14 s⁻¹, t12G=0.018 s⁻¹, and t12U=0.003 s⁻¹ for let-7a and t12A=0.027 s⁻¹, t12C=0.013 s⁻¹, and t12G=0.019 s⁻¹ for miR 21), even though the t12 mismatch is closer to the cleavage site.

Surprisingly, some mismatches near the cleavage site enhanced cleavage. The rate of cleavage of a target bearing t12A mismatched with the let-7a g12G was 2.5 fold faster than the fully complementary t12C target, which had a cleavage rate of 0.055 s−1 (FIG. 7B). The let-7a target bearing a t12A mismatch was cleaved at the fastest rate (FIG. 7B, let-7a diagonal), and the t12A mismatch was present in many doubly mismatched targets that cleaved at the fastest rates. In fact, the cleavage rates of 37 of the 60 doubly mismatched targets containing a t12A mismatch were faster than the fully complementary target (FIG. 7B). These data suggest that mismatches flanking the cleavage site reduce strain in the RNA duplex and allow the target to more easily assume a cleavable orientation. Counterintuitively, a t12 mismatches that weakened the affinity of let-7a RISC for target (FIG. 5A) showed the greatest enhancement in cleavage rates (FIG. 7B): changing the t12C:g12G base pair to a t12A (0.14 s⁻¹) increased the cleavage rate while a 12G substitution (0.018 s⁻¹) and a t12U substitution (0.003 s⁻¹) decreased the cleavage rate relative to the fully complementary let-7a target (0.055 s−1). miR 21 RISC showed the same trend: t12U>A (0.027 s⁻¹) or t12U>G (0.019 s⁻¹) slowed the rate of cleavage less than t12U>C (0.013 s−1) relative to the fully miR 21-complementary t12A target (0.087 s−1; FIG. 7B). Moreover, seed mismatches were surprisingly well tolerated, with the majority of seed mismatches having small effects on single turnover cleavage rates (FIG. 7B). This was unexpected given the importance of seed complementarity for RISC binding. For let-7a, mismatches at position t5 accelerated cleavage. And for both let-7a and miR 21, the rate of cleavage was unchanged for t8G or t7C mismatches. Thus, an siRNA fully complementary to its target is unlikely to be optimal, perhaps because specific mismatches enable the RISC:target complex to more fully populate the catalytically competent conformation.

### Example 10

### Target Mismatches to the Guide RNA 3′ End Accelerate Single-Turnover Cleavage Rates

Target:guide mismatches at the 3′ end of the guide (g17-g21) increase the rate of multiple turnover target cleavage, a phenomenon hypothesized to reflect faster release of the cleaved products (Tang et al., 2003; Haley and Zamore, 2004; Wee et al., 2012). In the experiments, such mismatches also increased the rate of single-turnover cleavage, suggesting that unpairing the guide 3′ end lowers the barrier to RISC adopting a cleavage-competent conformation (FIGS. 7B-7D). Remarkably, single, double, and triple mismatches from t15-t21 for miR-21 and t16-t21 for let-7a increased the single-turnover cleavage rate (FIGS. 7B-7D) even though some decrease binding affinity. Even when nucleotides t15-t21 (miR 21) or t16-t21 (let 7a) were all simultaneously mismatched with the guide, the cleavage rate increased (FIG. 7C). It is noted that bases t17-t21 have not been directly observed in any structures of target-bound RISC. The observation that individual or contiguous mismatches at these positions enhance cleavage provides strong evidence that these nucleotides can form canonical base pairs before the target is cut.

### Example 11

### Guide and Target Bulges Have Different Effects on Cleavage Kinetics

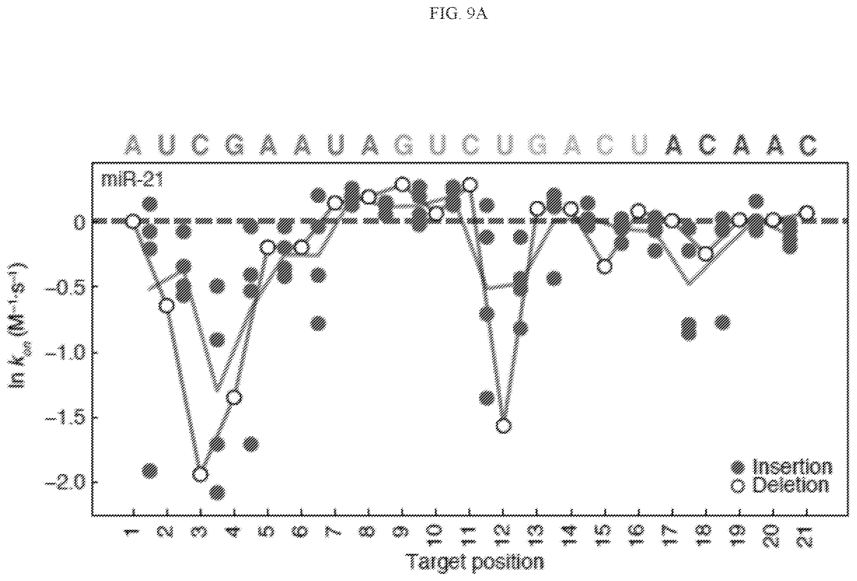

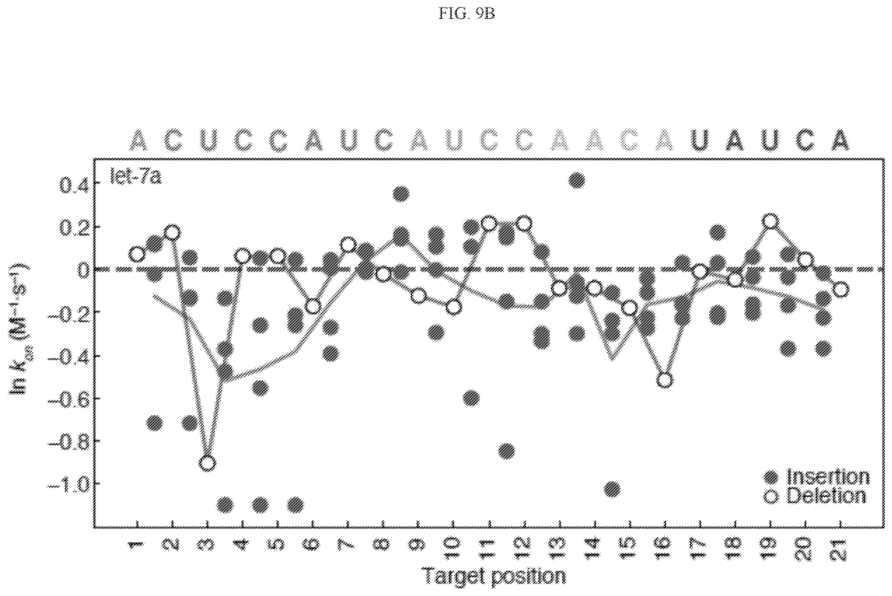

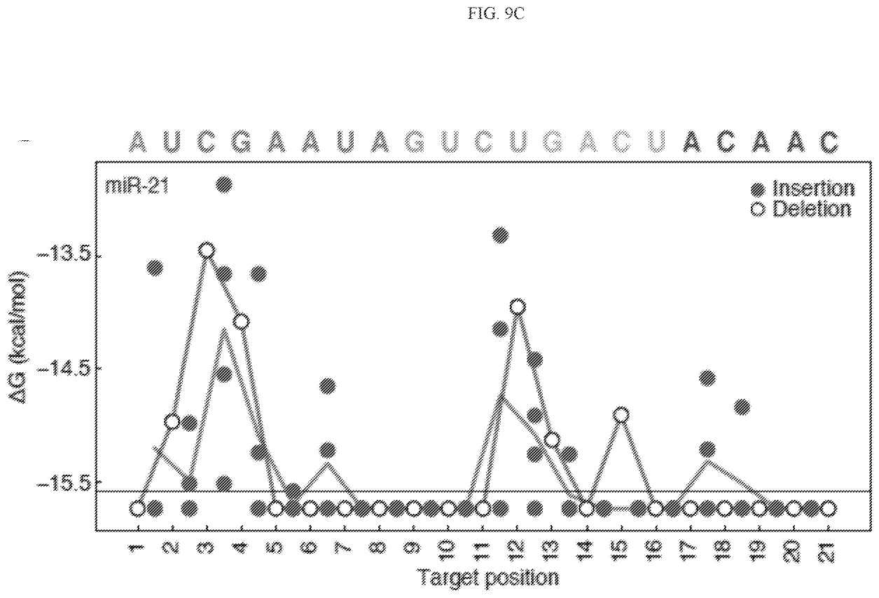

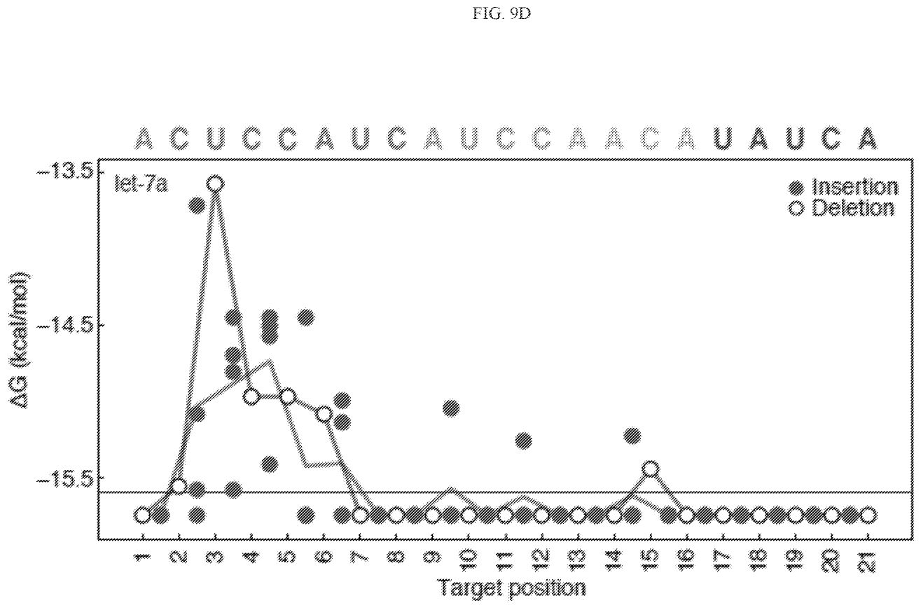

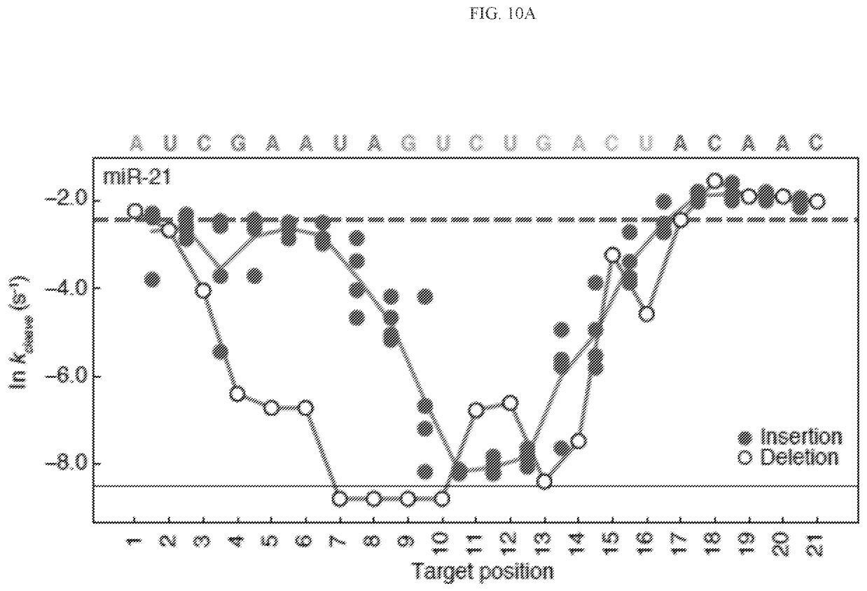

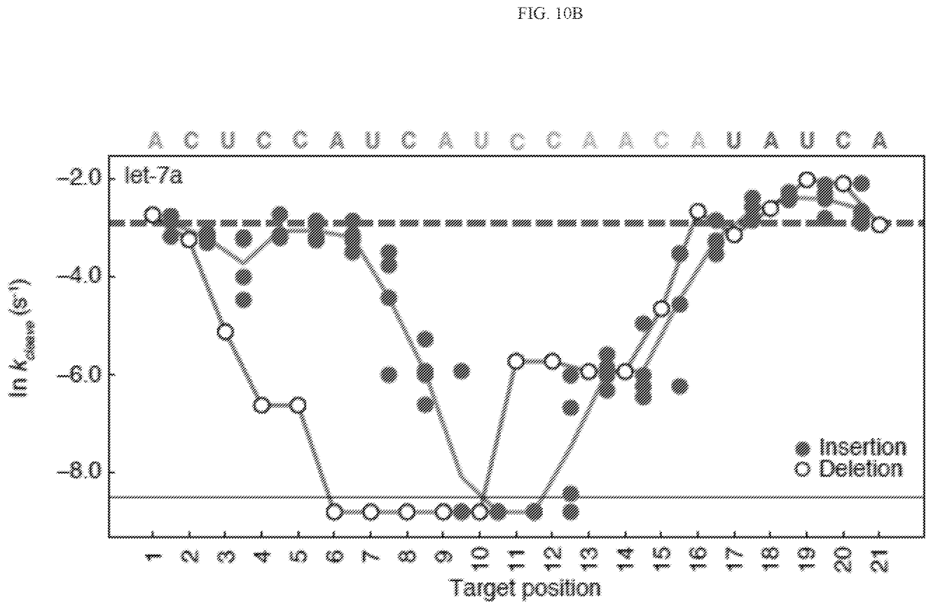

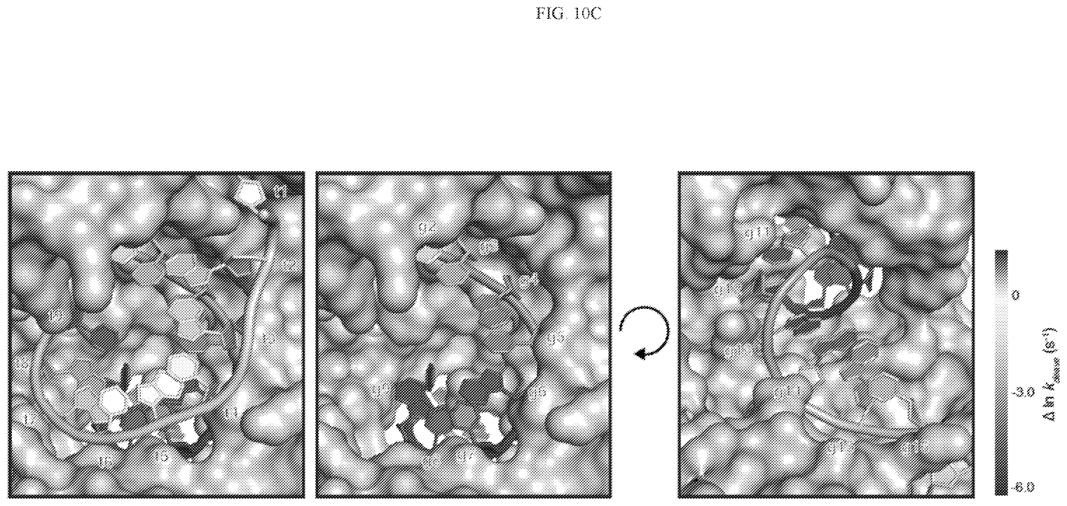

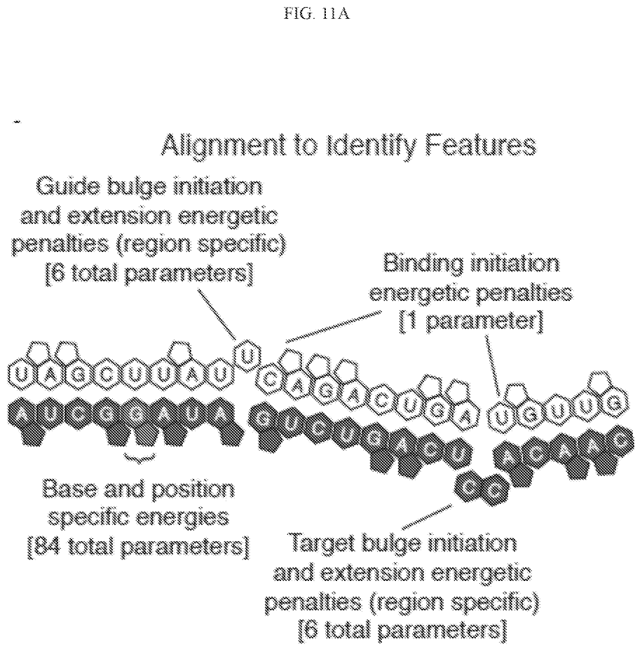

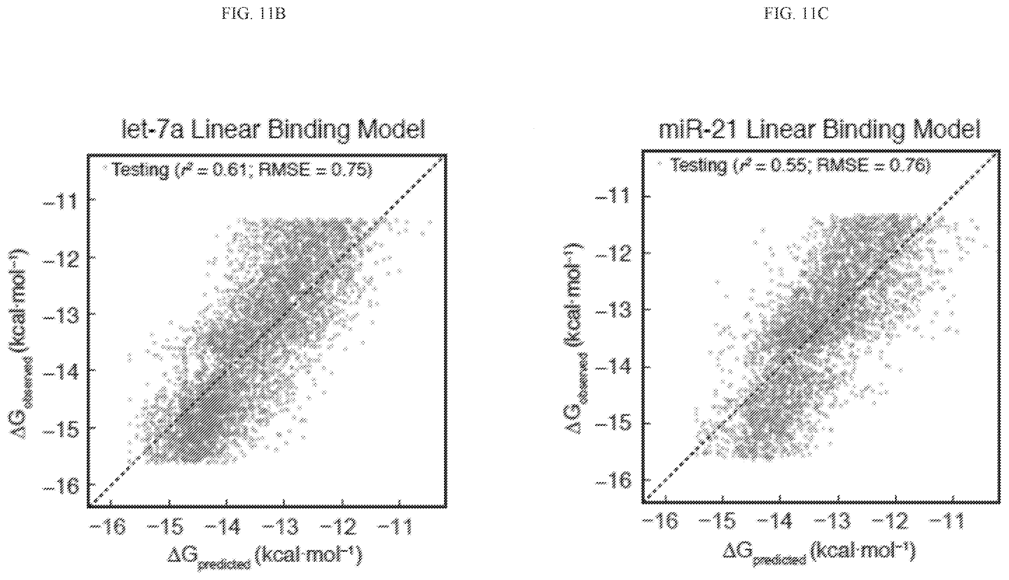

In the context of a fully complementary sequence, single insertions and deletions (indels) had similar effects on RISC association rate and binding affinity. Indels that disrupted seed pairing resulted in markedly slower association and less stable binding, whereas indels in the center and distal 3′ end of the target were well tolerated (FIGS. 9A and 9B). In contrast, single insertions and deletions at the same target position had markedly different effects on the single turnover cleavage rate for both miR 21 and let 7a (FIGS. 10A and 10B). Insertions disrupted cleavage in a similar manner to mismatches: single insertions that disrupted pairing between central bases resulted in nearly undetectable target cleavage (<0.0002 s⁻¹ for let-7a and <0.001 for miR 21 s⁻¹), insertions in the seed slightly lowered the cleavage rate, while insertions opposite the distal 3′ end of the guide enhanced cleavage. In contrast, single-nucleotide target deletions from t3 to t5 substantially reduced cleavage rates relative to the fully complementary target. Deletion of nucleotide t6 for let-7a or t7 for miR-21 eliminated detectable cleavage. Between positions t4 and t8, the cleavage rates for single-nucleotide target deletions were consistently >30 fold slower than either of the neighboring insertions. Interestingly, targets bearing single-nucleotide deletions at t11 were readily cleaved. Little or no cleavage was detected for targets bearing mismatches or single-nucleotide insertions at these very same positions.

Mapping the let-7a perturbations onto the AGO2 RISC crystal structure (Schirle et al., 2014) suggests an explanation for the distinct effects of insertions and deletions on RISC function (FIG. 10C). Target insertions are expected to require looping the inserted base out of the RNA duplex to maintain pairing to the RISC-bound guide RNA. In contrast, target deletions are predicted to require looping out of the unpaired guide base to accommodate extensive pairing to the target. The structure predicts that because AGO2 constrains the seed nucleotides of its guide RNA, looping out an extra guide base is sterically prohibited. It is anticipated that this restriction in guide geometry forces the extra guide base to be stacked into the duplex, as was observed in crystal structures of DNA target-bound TtAgo with DNA guide bulges (Sheng et al., 2017). Accommodating the extra guide base in the RNA duplex likely distorts the cleavage site, preventing efficient cleavage of these targets. By base g10, the guide backbone has begun to exit the central cleft of AGO and is facing solvent. Guide bulges at this point begin to have a smaller effect on the cleavage rate, suggesting that the extra guide base can now loop out of the duplex without disrupting the cleavage site. By contrast, the RNA target experiences steric constraint by AGO2 at different positions in the duplex. Target bases pairing to the seed region have a solvent-facing backbone and are suspected to be more capable of looping a single unpaired base out of the duplex. As the target strand passes through the central cleft of the protein, however, the target backbone begins to abut the PAZ domain of the protein and becomes more sterically constrained. This region, which also contains the cleavage site, is where target insertions become the most perturbative to cleavage. These findings highlight the structural sensitivity of AGO2 cleavage activity, demonstrating that helical imperfections well outside the cleavage site can have significant effects on cleavage activity, even when they minimally modulate RISC binding.

### Example 12

### Models for RISC Binding Affinity and Cleavage Kinetics

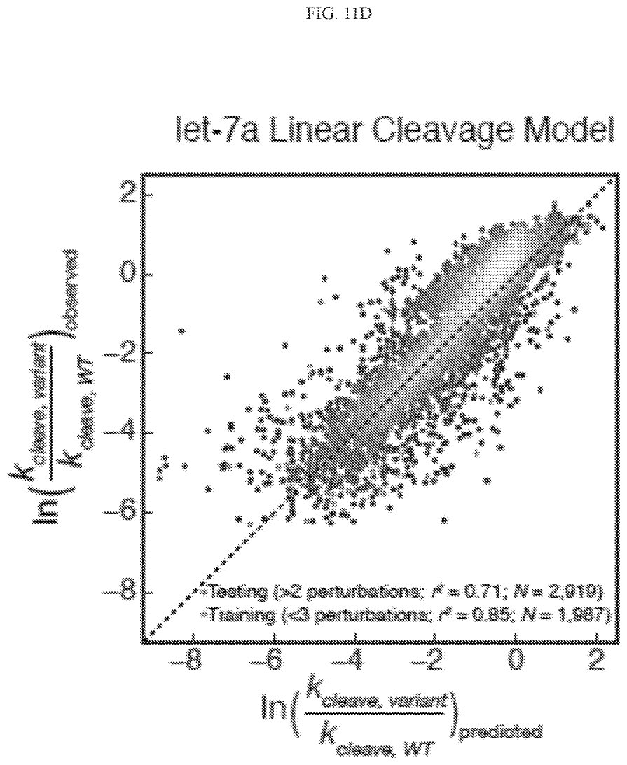

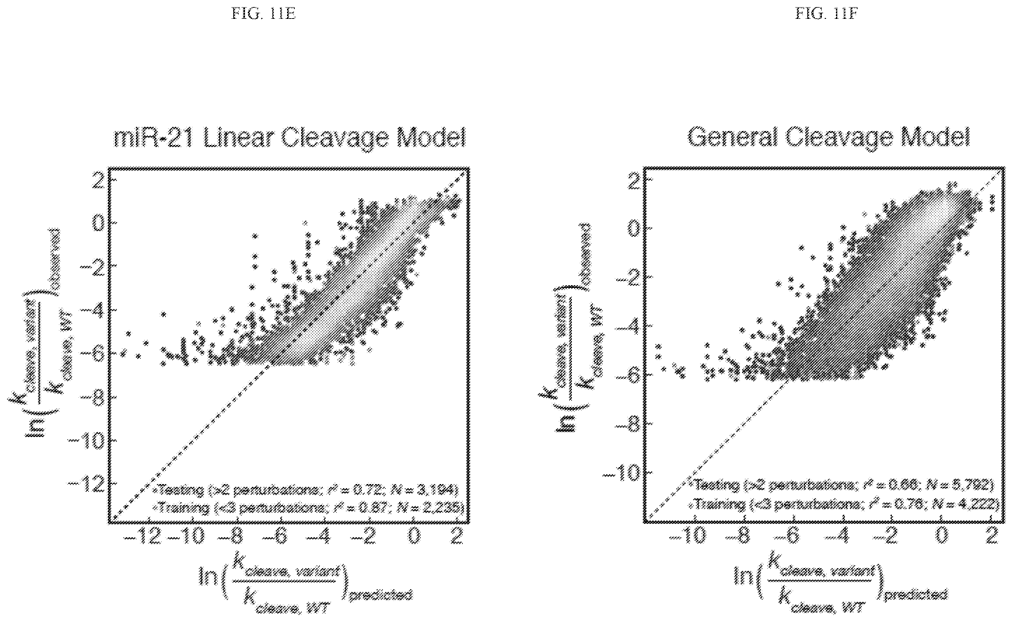

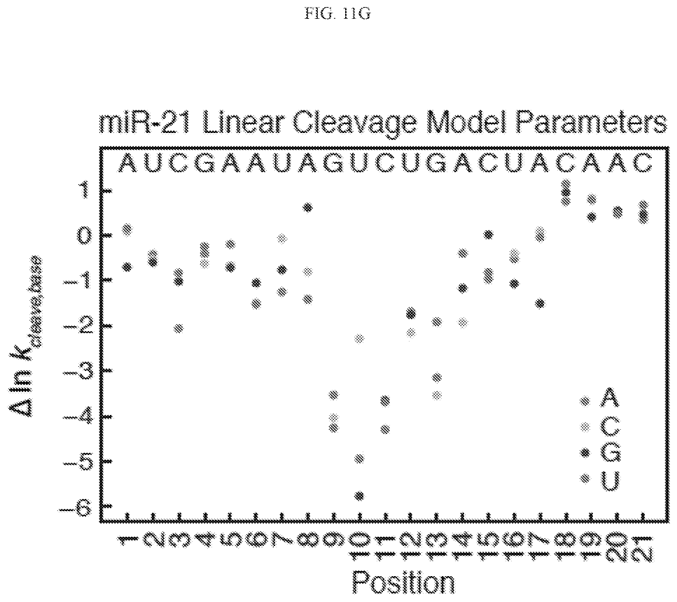

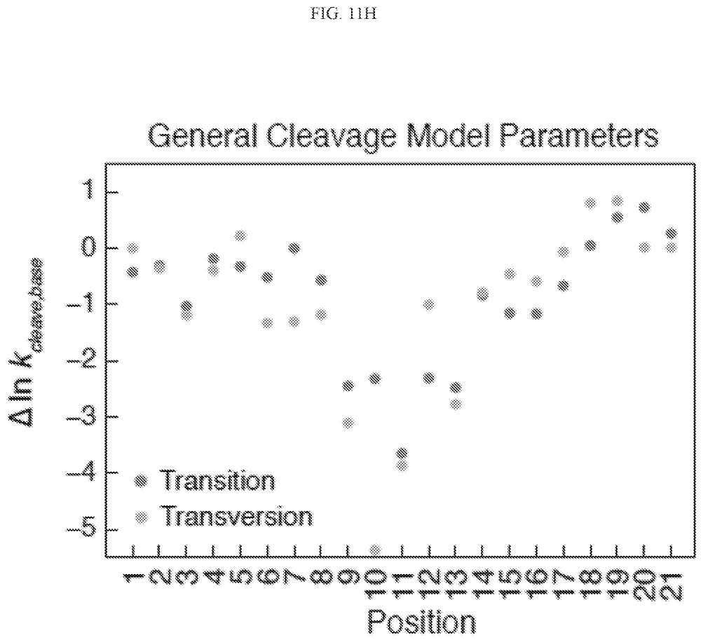

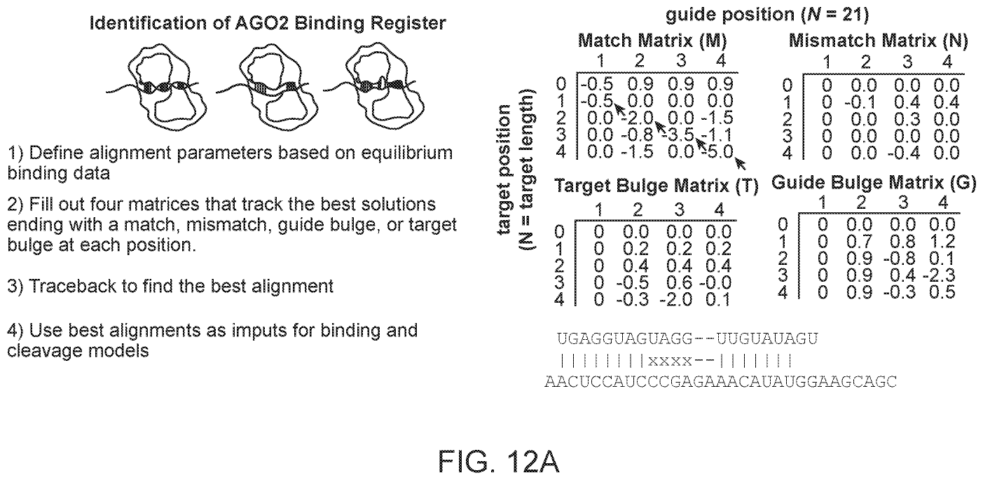

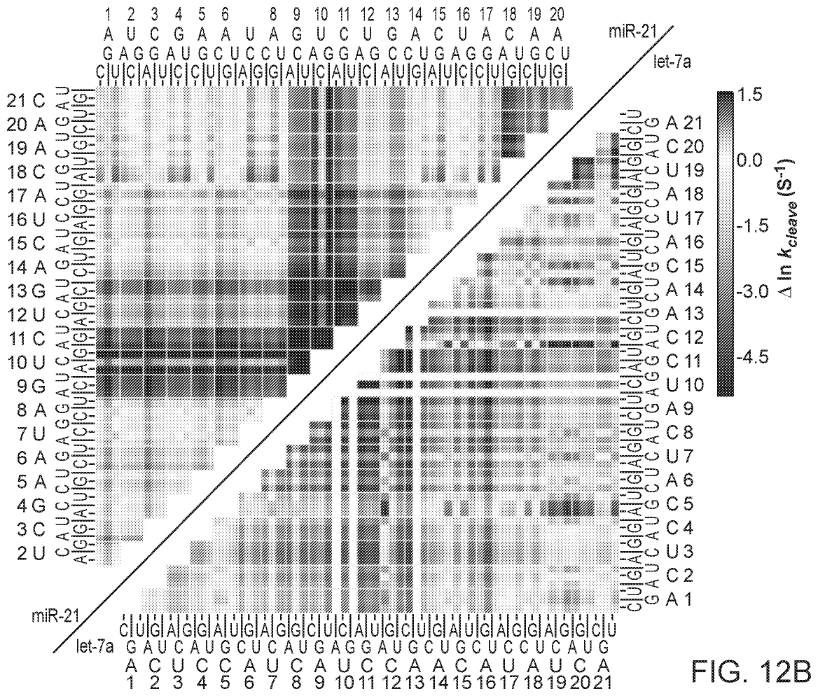

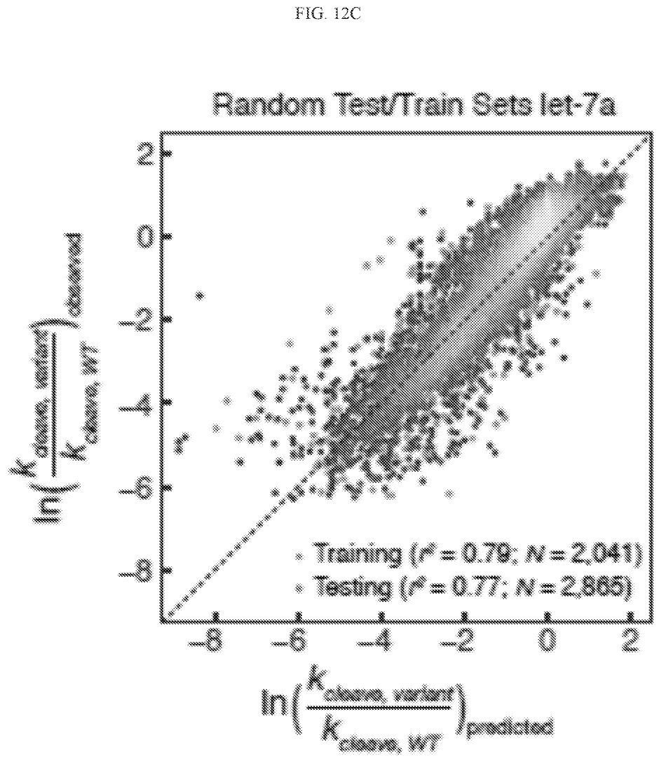

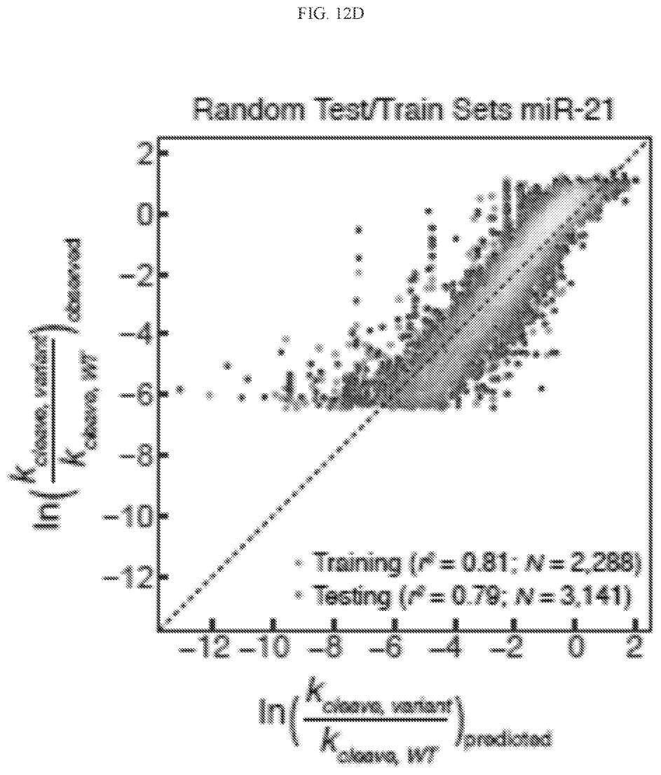

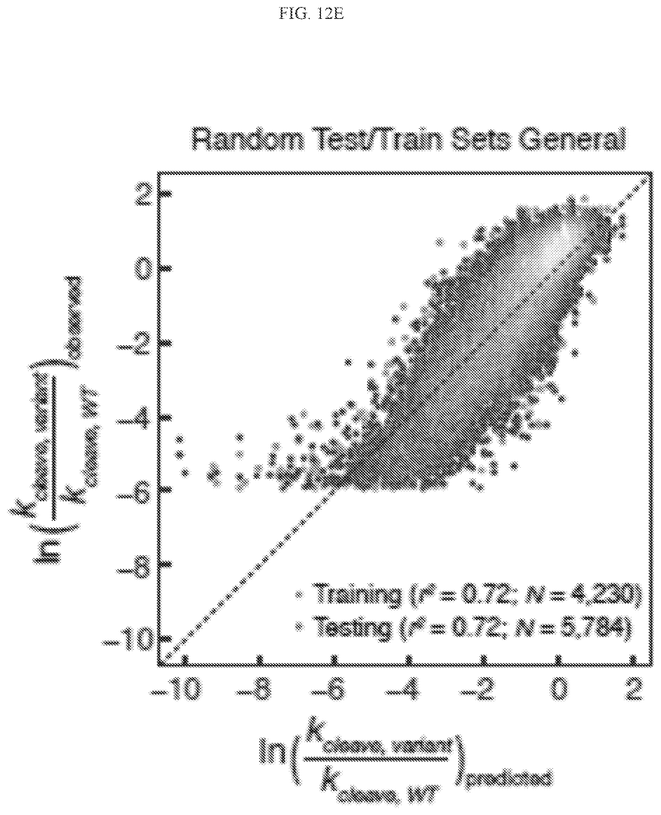

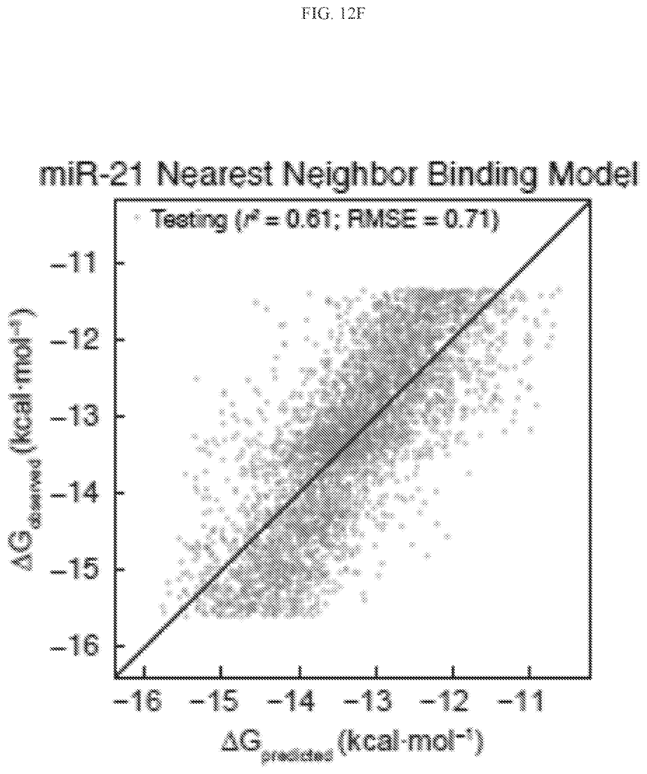

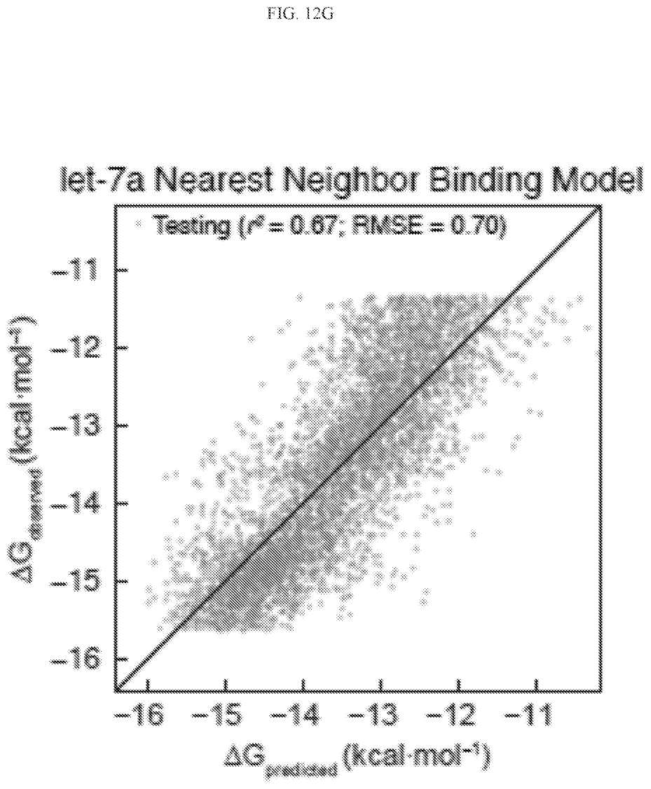

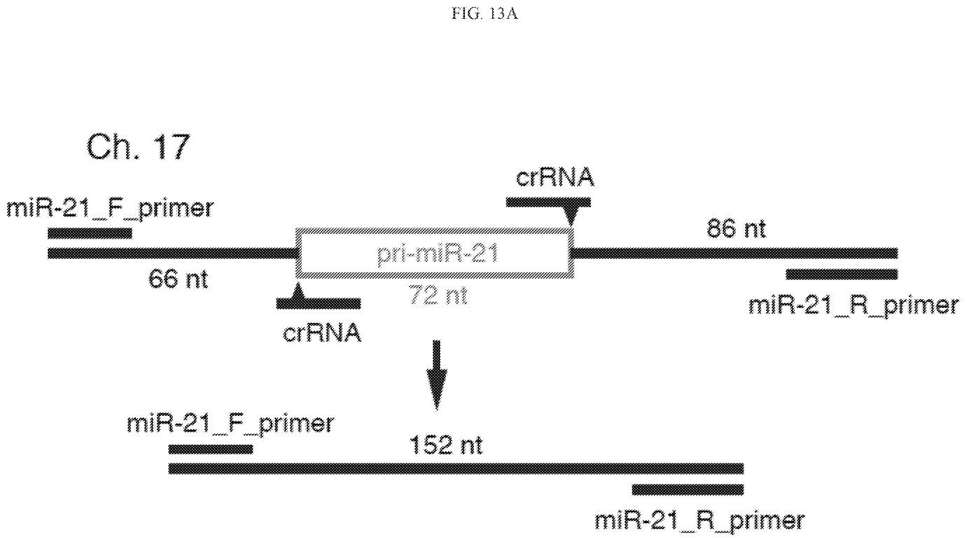

To predict binding affinity and cleavage rates of any miR-21 or let-7a RISC target, binding and cleavage were modeled separately for each guide. A dynamic programming alignment algorithm enabled determination of the expected binding register for each potential cleavage target (FIG. 11A and FIG. 12A). To model binding affinity, we included one energy parameter for each base at each position (21 positions×4 nucleotide identities=84 total parameters). Because the effect of insertions and deletions depends primarily on their position—seed, central, or 3′ supplemental—and whether they perturb the guide or target strand, bulge opening and extension penalties were included for each of these three regions for each strand (3 regions×2 parameters (opening and extension)×2 strands=12 bulge parameters). A base-pairing initiation term and a term to account for internal structure formed by the RNA targets were also included. Half the data was used to train the model and the remaining, randomly selected data was used to test the model. This simple, linear energetic model predicts 61% of the variance in binding affinity for let-7a and 55% for miR-21 RISC (FIGS. 11B and 11C). More complicated models performed only marginally better (FIGS. 12F and 12G).

Next, an appropriate set of parameters and overall architecture for the cleavage model was defined. Because double-mismatch cleavage rates are predicted well by single-mismatch cleavage rates (FIG. 12B), a linear model consisting of parameters for each mismatched base at each position (3 mismatched nucleotide identities×21 positions=63 parameters) was developed. Insertions of different nucleotides had similar effects at most positions, allowing the use of single parameters for any base insertion at any given position (FIGS. 8H and 8I). Increasing the length of target bulges led to a decreasing cleavage rate from positions t1-t11, but had no effect on the cleavage rate, and in some cases actually increased the cleavage rate at positions at the 3′ end of the guide (t12-t21; FIGS. 8H and 8I). As a result, target bulge parameters scaled with the size of the target bulge (penalty×bulge size) for bases t1-t11, and were identical for any bulge length (e.g. penalty for 1 nt bulge=penalty for 2 nt bulge) from positions t12-t21 (20 total target bulge parameters). For guide bulges, effects were generally additive, and, in most cases, if multiple guide bulges were present no cleavage was observed (FIGS. 8E and 8G). To account for this, guide bulge penalties for each position from t2-t20 (19 total guide bulge parameters) were included. A model containing these 102 free parameters was fit to targets containing single and double mismatches and single insertions and deletions for both let-7a (1,766) and miR-21 (2,084).

The cleavage rates of triple mismatch targets and targets containing multiple insertions and deletions that were not used to train the model (2,361 for let-7a and 2,765 for miR-21) were predicted. This model fit well to the data collected on targets with 2 or fewer mismatches, insertions, or deletions and quantitatively predicted the cleavage rates of targets containing greater than two perturbations with high accuracy (r2=0.71 and 0.72, respectively; FIGS. 11D and 11E). The model estimated that mismatches at t8 led to a 1.9 fold increase in cleavage rate and that mismatches at the 3′ end of the guide accelerate the cleavage rate up to 2.4 fold (FIG. 11D).

Given that the cleavage rates for targets of each guide can be accurately predicted, and that similar qualitative behaviors for cleavage by RISC when loaded with either let 7a or miR 21 was observed, a generalizable model to predict the cleavage rate of any RISC complex was constructed. To constrain the number of free parameters of this model, energetic penalties only for transitions or transversions at each position were considered, along with target and guide bulge parameters as described above. This model (81 free parameters) was fitted to single and double mismatch targets and single target insertions and deletions of both let-7a and miR-21 (3,850), then predicted the cleavage rates of targets of each guide containing triple mismatches or multiple insertions or deletions (5,126; FIG. 11E). This model was able to accurately predict cleavage of targets containing multiple mismatches, insertions, or deletions across both guide sequences (r2=0.66). The parameters obtained from this fit give insights into the physical constraints on RISC cleavage. Most notably, transversions perturbed cleavage more than transitions at positions t6-t11, but transversions perturbed cleavage less for all but one base at positions t12-t19 (FIG. 11H). Mismatches that disrupt RISC structure at positions t6-t11 may not be easily accommodated, and their perturbations may propagate to the cleavage site. Supporting this view, guide bulges were more disruptive when they were 5′ to the cleavage site (FIGS. 10A-10C). Conversely, at positions 3′ of the cleavage site, transversions were often preferred and likely increase the ability of the RISC ternary complex to obtain a cleavable conformation relative to more readily accommodated transversions.

### Example 13

### RISC Kinetic Parameters Predict Knockdown in Cells

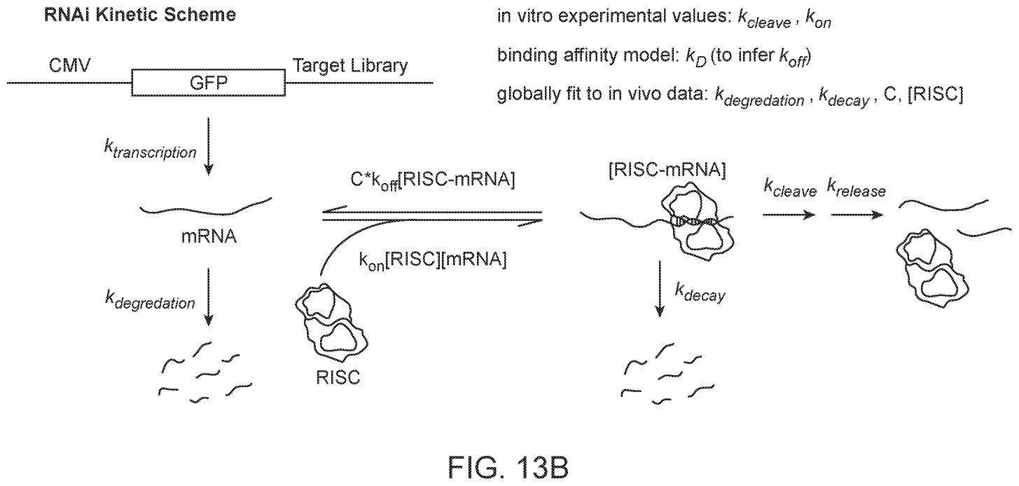

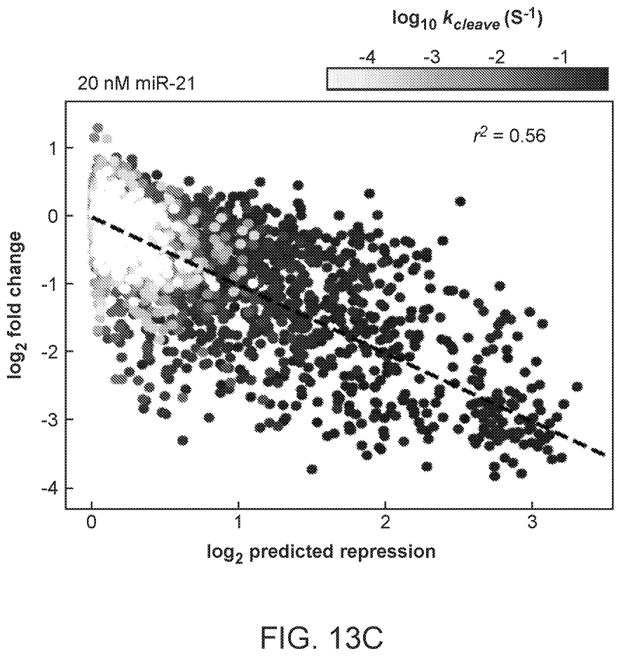

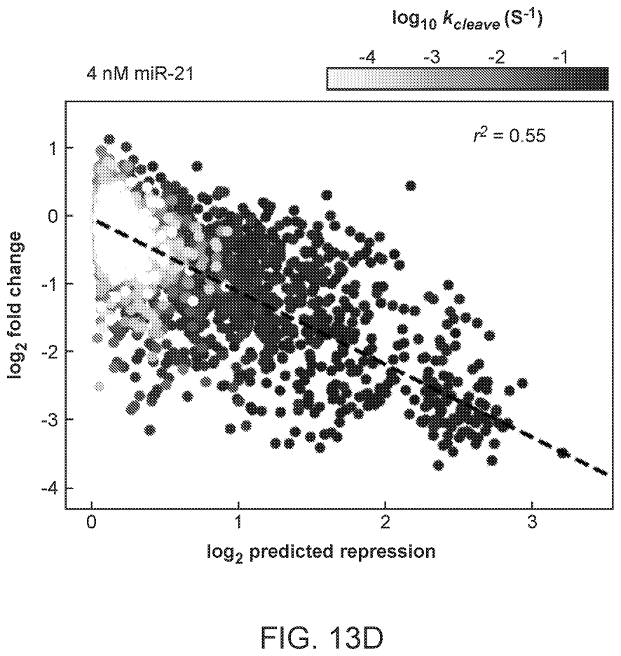

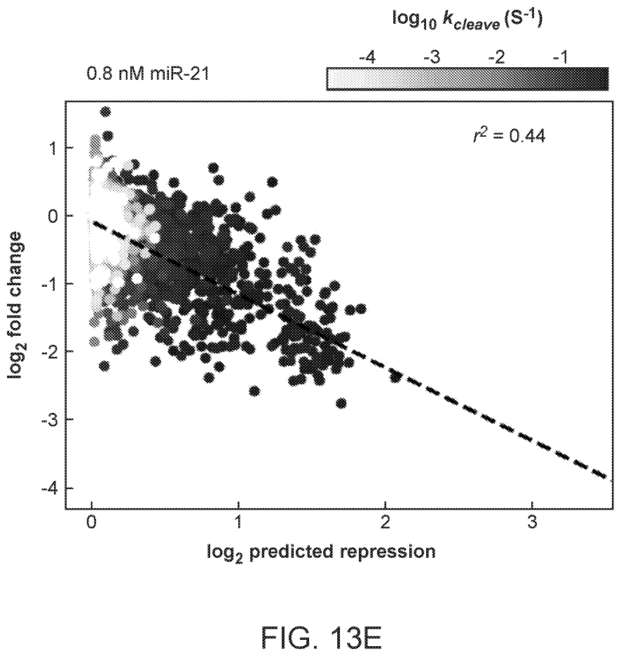

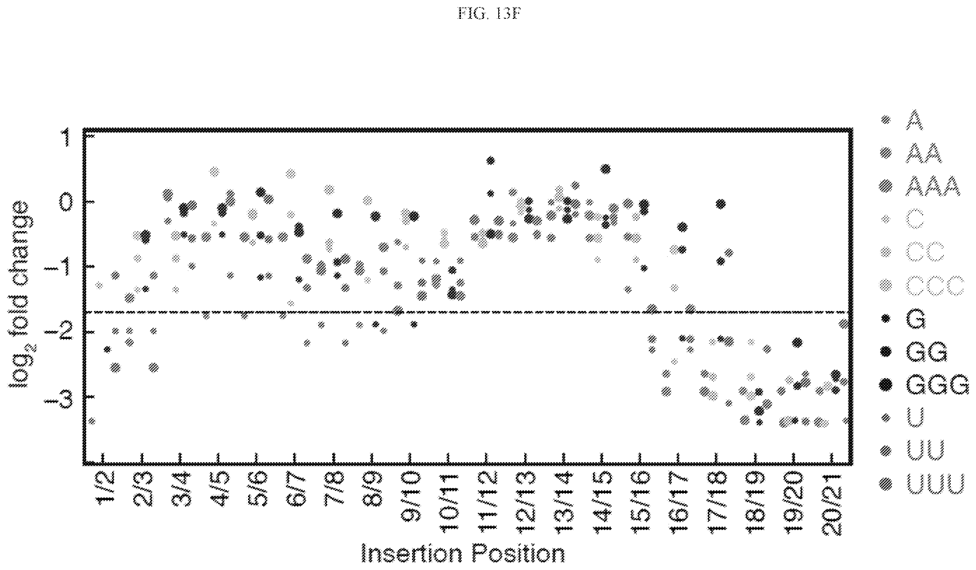

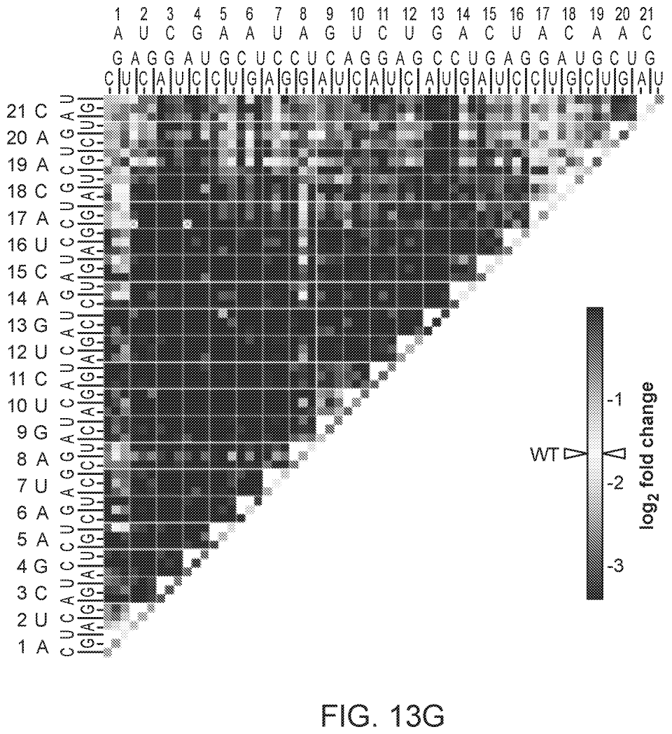

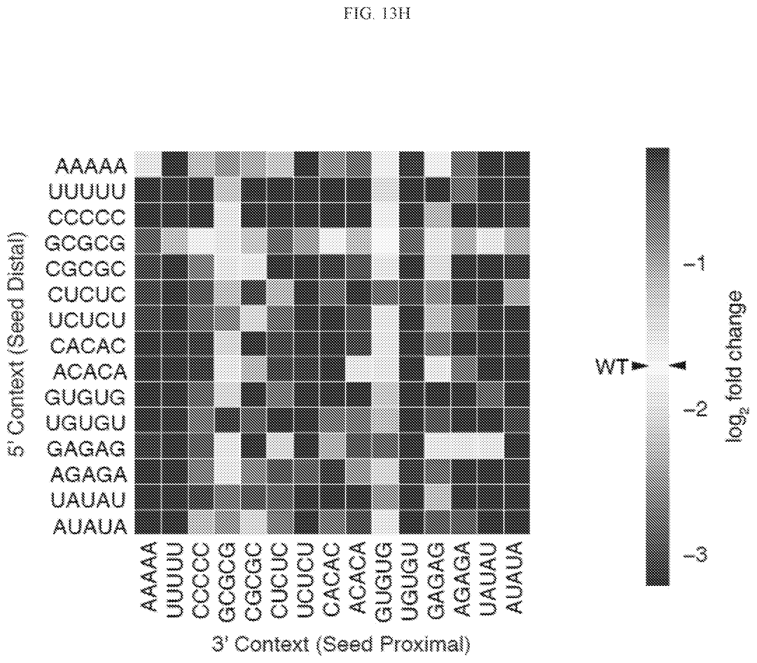

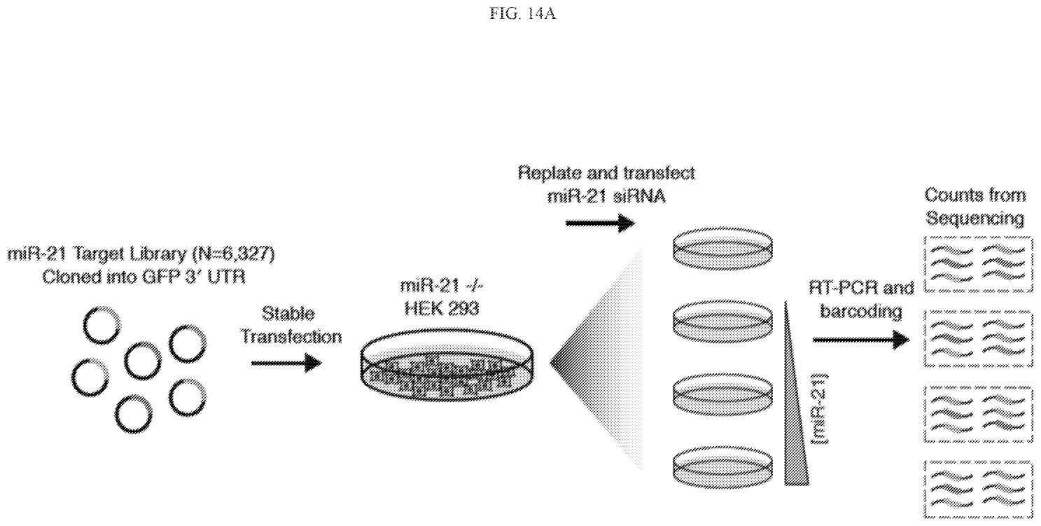

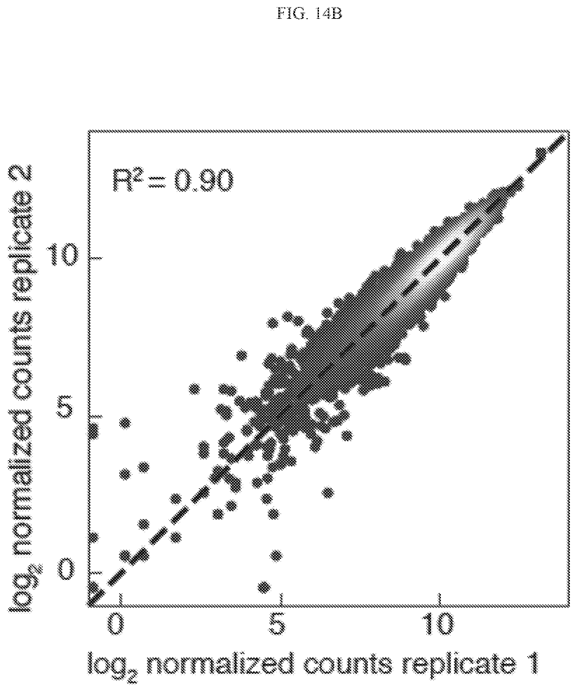

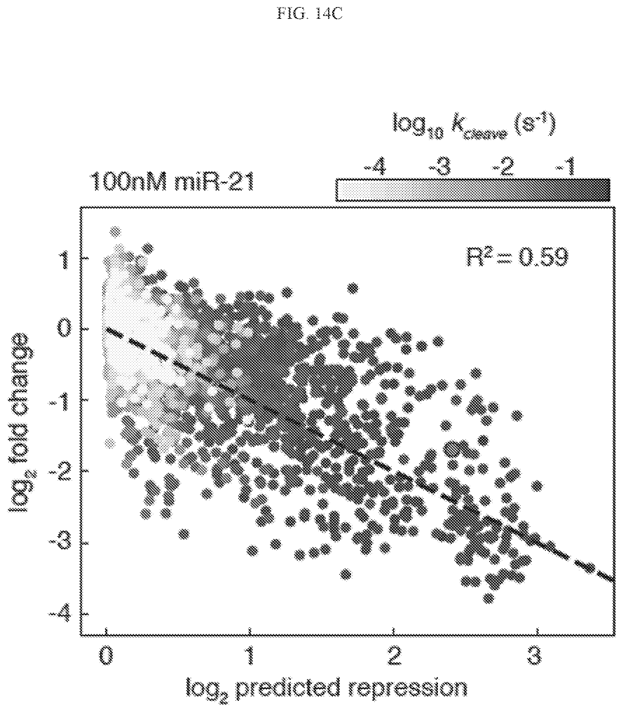

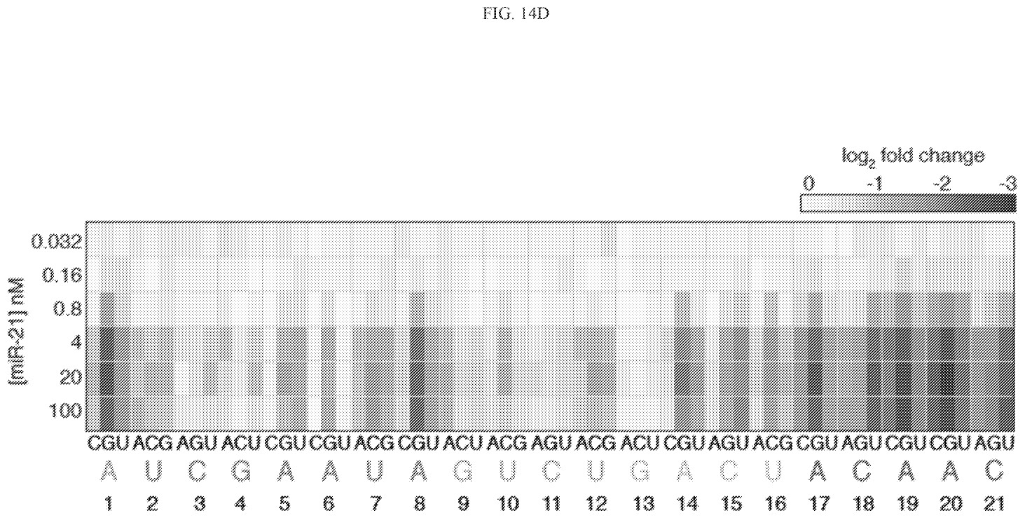

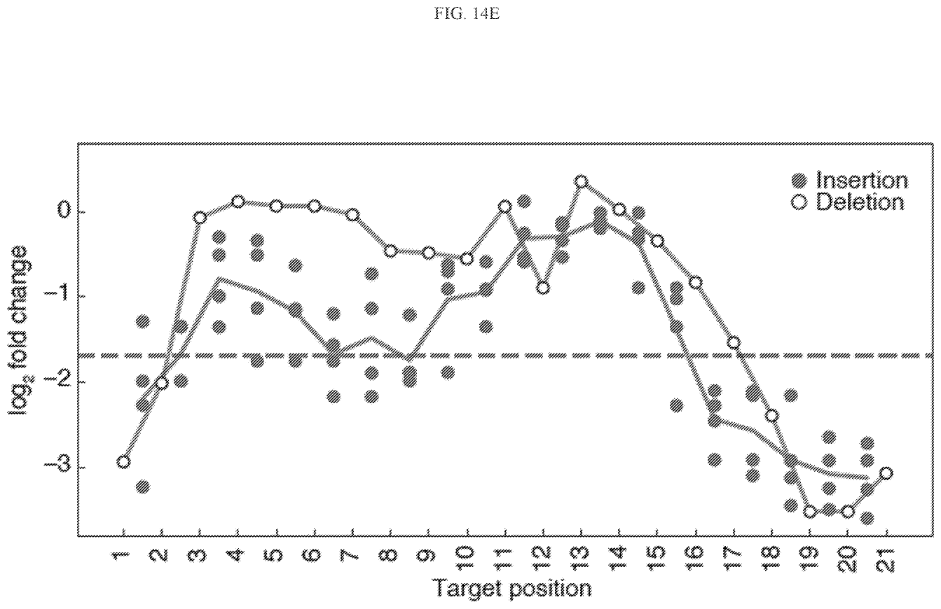

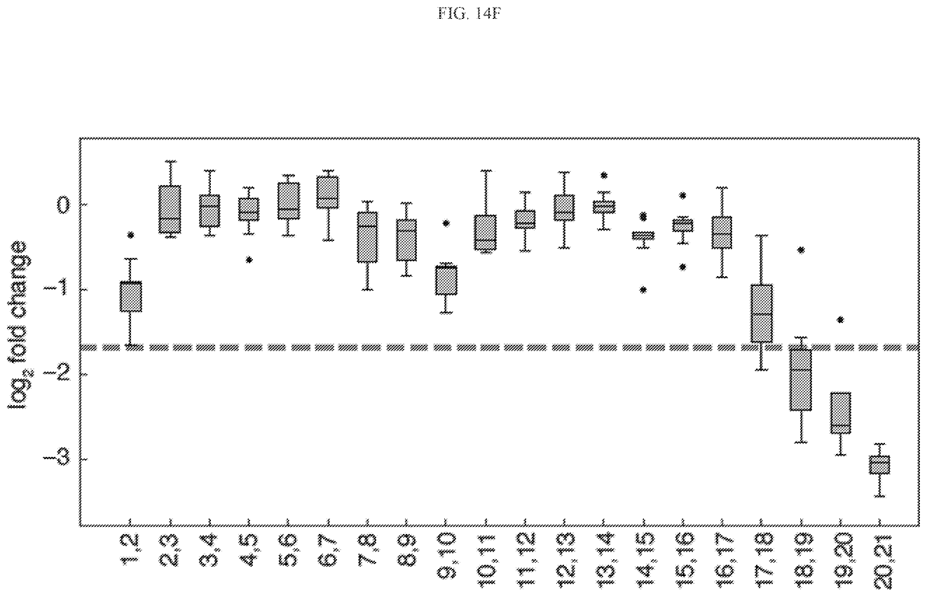

To determine how well the in vitro measured biochemical parameters predict siRNA efficacy in cells, a cellular system for measuring the change in abundance of thousands of miR-21-complementary targets in parallel was generated. First, we used paired CRISPR-Cas9 guides to knockout the entire pri-miR-21 hairpin in HEK-293 Flp-In T-REx cells (FIG. 13A). A subset of the miR-21 target library (the 6,327 sequences used in the cleavage experiments) was cloned into the 3′ UTR of an eGFP reporter plasmid and used the piggyBac transposon system to stably integrate this library into the miR-21 knockout cell line. Increasing concentrations of a miR-21 siRNA were transfected and RNA was isolated from cells after 48 h. Using primers flanking the cleavage site, sequencing libraries were generated from replicates of each treatment condition. After normalizing for sequencing depth, the change in steady-state target abundance as the fraction of counts from each miR-21 transfection relative to the counts from the mock transfected miR-21 knockout line was calculated (FIG. 14A).

siRNA efficacy was predicted to reflect each target's RISC association, dissociation, and cleavage rate, as well as the free RISC concentration, the basal mRNA decay rate, and the miRNA-accelerated decay rate. A biochemical model was derived to predict the change in target abundance at steady-state based on these parameters (FIG. 13B). Because the true dissociation rate for extremely high affinity (K_{D}<10 pM) targets in vitro was not able to be measured, dissociation rates were used to determined by multiplying the predicted affinity values by the measured association rates (k_{off}=K_{D}×kₒₙ) to model target reduction. The free RISC concentration was fit as a constant for all targets for each miR-21 transfection concentration. Because all reporter constructs had essentially the same 3′ UTR length and sequence composition, the basal mRNA decay rate and miRNA-accelerated decay rate were assumed to be constant for all targets, and these parameters were fit globally across all transfection conditions. Unlike RISC-CNS cleavage rates, the flanking context of targets significantly influenced target knockdown in cells (FIG. 13H), likely due to the greater length of flanking sequence, which is predicted to increase the formation of competing secondary structures or binding of cellular proteins. For this reason, only targets containing five adenosines flanking the target region were used in model fitting and subsequent analyses (4,483 sequences). While this does not eliminate differential effects of structure or other RNA binding proteins on the targets examined, it does reduce their likelihood of confounding comparative analyses. Many variants with RISC-CNS cleavage rates >10 fold faster than their estimated dissociation rates showed little change in abundance in cells, suggesting that the dissociation rate in cells is much faster than the dissociation rate measured in vitro. This could reflect differences in ionic environment or the activity of RNA helicases or other RNA-binding proteins. To account for this discrepancy, a single dissociation-rate scaling term was fitted for all targets across all treatment conditions. This model performed well for each of the three highest miR-21 transfection conditions (R²=0.59, 0.56, 0.55; FIG. 14C and FIGS. 13C-13E).

Next, it was examined more closely the effect of single-nucleotide mismatches on knockdown in cells (FIG. 14D and FIG. 13G). In agreement with RISC-CNS results (FIG. 7B), mismatches at target position t13 resulted in less target reduction than most seed mismatches, highlighting the importance of pairing at this position for efficient target cleavage. Several types of mismatches at positions t6 (g6U:g6G mismatch), t7 (g7A:g7C mismatch), and t8 (g8U:g8G mismatch) and, to a lesser extent, t10 (g10A:g10C mismatch) and t12 (t12A:g12C mismatch) reduced target abundance more than neighboring seed or central mismatches, underscoring the finding that continuous base pairing in the AGO active site is not required for target cleavage. Mismatches from t17-t21 resulted in target reduction equal to or greater than that observed for the fully complementary target, in good agreement with the RISC-CNS finding that mispairing at these positions enhances the rate of target cleavage. Similarly, the effect of target deletions was predicted by RISC-CNS: deletions in the seed-matching region yielded little or no target knockdown in cells, while single target insertions between t2 and t9 resulted in similar or slightly less knockdown than the fully complementary target (FIG. 14E). Knockdown of targets containing either single insertions or deletions after position t12 was similar. Notably, 3′ distal indels resulted in 2-4 fold greater siRNA efficacy compared to the perfectly complementary target in the same context. Paradoxically, single-nucleotide insertions in the seed-matching sequence of the target caused only a modest reduction (<2 fold on average) in siRNA efficacy relative to the fully complementary sequence, yet, as RISC-CNS predicted, the insertion of two or more bases in the seed binding region reduced siRNA efficacy, with all two and three nucleotide insertions constrained to be between positions 3 and 7 causing less than a 40% decrease in mRNA levels (FIG. 13F). However, insertion of 1-3 nucleotides at target positions t11-t15 reduced RISC activity a similar amount (<2 fold) regardless of the length of the insertion, consistent with the RISC-CNS findings (FIGS. 8H and 8I).

Finally, the library of targets included all 180 possible tandem double mismatches (FIG. 14F). As predicted by RISC-CNS, target mismatches with the last two guide nucleotides enhance target cleavage: the abundance of all nine miR 21 targets bearing t20t21 mismatches was lower than that observed for the fully complementary target. Targets bearing tandem mismatches in the cleavage site, particularly t9t10, were better RISC substrates than targets with tandem mismatches in either the seed or 3′ supplemental region. Yet such t9t10 mismatched targets—which bind RISC with high affinity (K_{D}<10 pM)—were not cleaved in RISC-CNS experiments. A likely explanation for this paradoxical result is that, in cells, targets bearing central, tandem mismatches are substrates for miRNA-mediated transcript destabilization rather than cleavage (Hutvágner and Zamore, 2002; Zeng et al., 2002; Doench, 2003); RISC-CNS only reports on Argonaute-catalyzed cleavage.

### Example 14

### Methods

Library design. Target libraries for let-7a and miR-21 loaded RISC were designed to include all single mismatches, all double mismatches, a subset of triple mismatches, all single target insertions and deletions, all target insertions of 2-7 identical nucleotides, pairs of 2-5 consecutive transitions or transversions, four way combinations of two consecutive transitions or transversions (eight total mismatches), stretches of mismatches to the complement target base of all lengths throughout the target sequence, the top 1000 predicted targets from four algorithms (TargetScan, Diana-microT, miRanda-mirSVR, and PicTar2), and targets identified with the CLASH experimental method. Each designed target was placed within context sequence that typically consisted of five flanking adenosine nucleotides on the 5′ and 3′ ends of the target. The predicted targets were included with the 5 flanking nucleotides present around the actual target sequences. Targets identified from CLIP experiments in mice (Chi el al., 2009) were also included, but the mm9 coordinates were lifted over to hg19 to identify the corresponding human targets, which were included in the library. Since these lifted targets were not experimentally determined, they were not used in comparing predicted targets (FIGS. 6A-6K) but were included to add more sequence diversity for model fitting. The perfectly complementary target was also placed in 225 distinct five nucleotide contexts, and the single mismatches were placed in four five nucleotide contexts to test for the effects of the flanking sequence. The perfectly complementary target was also placed in sequence contexts longer than five nucleotides that were designed to form RNA secondary structure with the target region. An overview of the library designs is shown in FIGS. 2A and 2B.

Assembly and Sequencing of library. Target libraries were synthesized by Custom Array (Bothell, Wash.) such that each variant was flanked by common 5′ and 3′ priming sequences. Predicted target variants were ordered with an alternate 3′ priming sequence so that these variants could be separated from the rest of the library. Ordered sequences ranged from 73 bp to 129 bp, and sequences shorter than the longest variant had random sequence appended until all variants were the same length. The first miR-21 library was ordered as a 12,000 oligonucleotide synthesis and contained 7,675 unique variants. The let-7a and second miR-21 libraries were ordered as part of two separate 92,000 oligonucleotide syntheses and contained 22,641 and 12,768 unique variants respectively.

Synthesized libraries were assembled into full constructs compatible with Illumina sequencing and with generation of RNA on chip (FIG. 2B). The assembly reactions were carried out in a 20 μl volume of 1×NEBNext Master Mix (NEB, M0541) with ˜10 pM of synthesized library, 10 pM of T7A1_stall, 50 pM of C_i7_bc_T7A1, 50 pM of either D_designed_lib_R2 or D_pTarget_lib_R2, and 250 pM of both C and D primers (oligonucleotide sequences available in Table 1). SYBR green was added at a final concentration of 0.6×to assembly reactions so that assembly progress could be monitored. Reactions were loaded into a QuantStudio qPCR thermocycler and went through cycles of 98° C. for 10 s, 63° C. for 30 s, and 72° C. for 3 sec until the SYBR green signal of a reaction began to plateau, after which the reaction was paused and that assembly reaction was removed. Assembly reactions ran between 14 and 19 cycles. Completed assemblies were purified using a QIAquick PCR purification kit, and a portion of the purified product was visualized on an agarose gel to confirm specific assembly of the intended product.

Assembled libraries were diluted and quantified against a standard library of PhiX (Illumina, Hayward, Calif.). PhiX standard was prepared by diluting stock PhiX to 200 pM in water and then serially diluting by 2-fold eight times, resulting in a standard curve that spanned 200 pM to 1.56 pM. Diluted libraries and the PhiX standard were amplified in qPCR reactions containing 500 pM primers (Illumina Adapter Sequences P5 and P7; Table 1) in 1×NEBNext Master Mix (NEB, M0541) with 0.6×SYBR green. Reactions were cycled at 98° C. for 10 s, 63° C. for 30 s, and 72° C. for 30 sec for a total of 25 cycles. Standards were run in duplicate, and all library samples were run in triplicate. Quantified libraries were then sequenced on an Illumina MiSeq instrument using custom read 1 and read 2 primers that flanked the variable region of each library sequence (Table 1). Libraries typically represented 10-20% of the total sequencing chip, with the rest of the chip comprised of high-complexity genomic libraries. Libraries were sequenced in two steps using paired end sequencing with 76-bp reads. Because library variable regions were all shorter than this read length, all variants were fully sequenced in both directions in each sequencing run.

TABLE 1

| | Sequence |
|---|---|
| | Passenger, guide in bold, seed, |
| RISC Loading | m indicates 2′-O-methyl ribose; p indicates 5′ monophosphate |
| Passenger strand for let-7a RISC | UAU ACA ACC UAC UAC CUG CUU (SEQ ID NO: 5) |
| Guide strand for let-7a RISC | pUGA GGU AGU AGG UUG UAU AGU-NH₂ (SEQ ID NO: 6) |
| Passenger strand for miR-21 RISC | UCA ACA UCA GUC UGA UAA GCU U (SEQ ID NO: 7) |
| Guide strand for miR-21 RISC | pUAG CUU AUC AGA CUG AUG UUG A-NH₂ (SEQ ID NO: 8) |
| RISC Purification and Activity | Sequence |
| Testing | RNA in italic, DNA; m, 2′-O-methyl; Bio, Biotin-6-carbon spacer |
| Capture Oligo to affinity purify | Bio-mAmUmA mGmAmC mUmGmC mGmAmC mAmAmU mAmGmC |
| let-7a RISC | mCmUmA mCmCmU mCmCmG mAmAmC mG (SEQ ID NO: 9) |
| DNA competitor to elute let-7a | CGT TCG GAG GTA GGC TAT TGT CGC AGT CTA T-Bio (SEQ ID |
| RISC | NO: 10) |
| Capture Oligo to affinity purify | Bio-mGmAmU mGmAmA mCmCmA mCmUmC mAmGmA mGmAmC |
| miR-21 RISC | mAmUmA mAmGmC mUmAmA mUmCmU mA (SEQ ID NO: 11) |
| DNA competitor to elute miR-21 | Bio-TAG ATT AGC TTA TGT CTC TGA GTG GTT CAT C (SEQ ID |
| RISC | NO: 12) |
| Forward primer to generate | GCG TAA TAC GAC TCA CTA TAG GGG TCC TTT GAT CGT GAC |
| templates for T7 transcription of | AAA ACA AT (SEQ ID NO: 13) |
| let-7a and miR-21 target RNAs | |
| Reverse primer to generate | CCC ATT TAG GTG ACA CTA TAG ATT TAT ACC TAG TTA AAC AGC |
| template for T7 transcription of | GGA ACT GTG TAT AAA AGG TTG AGG TAG TAG GTT GTA TAG |
| let-7a target | TAT CCA GAG GAA TTC ATT ATC AGT G (SEQ ID NO: 14) |
| Reverse primer to generate | CCC ATT TAG GTG ACA CTA TAG ATT TAC ATC TAG TTG AGG TGC |
| template for T7 transcription of | GGA ACT GTG TAT AAA AGG TTA GCT TAT CAG ACT GAT GTT |
| miR-21 target | GAA TCC AGA GGA ATT CAT TAT CAG TG (SEQ ID NO: 15) |
| Array Library Assembly Oligos | Sequence DNA |
| Illumina Adapter (P5) and T7A1 | AAT GAT ACG GCG ACC ACC GAG ATC TAC ACA CTG GTA TGC |
| promoter oligo | GAG ACG CAG GAT GNN NNN NNN NNN NNN NNA TTT ATC AAA |
| | AAG AGT ATT GAC TTA AAG TCT AAC CTA TAG GAT ACT TAC |
| | AGC C (SEQ ID NO: 16) |
| T7A1 promoter and stall sequence | ATT TAT CAA AAA GAG TAT TGA CTT AAA GTC TAA CCT ATA |
| | GGA TAC TTA CAG CCA TGT AGT AAG GAG GTT GTA TGG AAG |
| | ACG TTC CTG GAT CC (SEQ ID NO: 17) |
| Illumina Adapter (P7) and | CAA GCA GAA GAC GGC ATA CGA GAT CGG TCT CGG CAT TCC |
| designed library R2 sequence | TGC TGA ACC GCT CTT CCG ATC T (SEQ ID NO: 18) |
| Illumina Adapter (P7) and | CAA GCA GAA GAC GGC ATA CGA GAT CGA CGG CGT ACA CTT |
| predicted target library R2 | CTA TTC TGT CTT CCC GCG TCC G (SEQ ID NO: 19) |
| sequence | |
| Illumina Adapter (P5) | AAT GAT ACG GCG ACC ACC GAG ATC TAC AC (SEQ ID NO: 20) |
| Illumina Adapter (P7) | CAA GCA GAA GAC GGC ATA CGA GAT (SEQ ID NO: 21) |
| Array Library Sequencing | Sequence |
| Oligos | DNA |
| stall sequence R1 primer | ATG TAG TAA GGA GGT TGT ATG GAA GAC GTT CCT GGA TCC |
| | (SEQ ID NO: 22) |
| designed library R2 primer | CGG TCT CGG CAT TCC TGC TGA ACC GCT CTT CCG ATC T (SEQ |
| | ID NO: 23) |
| predicted target library R2 primer | CGA CGG CGT ACA CTT CTA TTC TGT CTT CCC GCG TCC G (SEQ |
| | ID NO: 24) |
| | Sequence |
| Array Experimental Oligos | DNA; 5Biosg, 5′ biotin IDT |
| 5′biotinylated roadblock for | /5Biosg/CAA GCA GAA GAC GGC ATA CGA GAT CGG TCT CGG CAT |
| designed libraries | TCC TGC TGA ACC GCT CTT CCG ATC T (SEQ ID NO: 25) |
| 5′biotinylated roadblock for | /5Biosg/CAA GCA GAA GAC GGC ATA CGA GAT CGA CGG CGT ACA |
| predicted target libraries | CTT CTA TTC TGT CTT CCC GCG TCC G (SEQ ID NO: 26) |
| antisense stall sequence | GGA TCC AGG AAC GTC TTC CAT ACA ACC TCC TTA CTA CAT |
| | (SEQ ID NO: 27) |
| 3′-Atto647N antisense stall | GGA TCC AGG AAC GTC TTC CAT ACA ACC TCC TTA CTA CAT |
| sequence | /3Atto647N/ (SEQ ID NO: 28) |
| Designed library R2 blocking | TCG GCA TTC CTG CTG AAC CGC TCT TCC GAT CT (SEQ ID NO: 29) |
| oligo | |
| Predicted target library R2 | CGA CGG CGT ACA CTT CTA TTC TGT CTT CCC GC (SEQ ID NO: 30) |
| blocking oligo | |
| T7 library construction oligos | Sequence DNA |
| T7 promoter and stall oligo | TAA TAC GAC TCA CTA TAG ATG TAG TAA GGA GGT TGT ATG |
| | GAA GAC GTT CCT GGA TCC (SEQ ID NO: 31) |
| Designed library R2 primer | CGG TCT CGG CAT TCC TGC TGA ACC GCT CTT CCG ATC T (SEQ |
| | ID NO: 32) |
| Predicted target library R2 primer | CGA CGG CGT ACA CTT CTA TTC TGT CTT CCC GCG TCC G (SEQ |
| | ID NO: 33) |
| Cleavage library sequencing | Sequence |
| oligos | DNA |
| stall sequence R1 primer | ATG TAG TAA GGA GGT TGT ATG GAA GAC GTT CCT GGA TCC |
| | (SEQ ID NO: 34) |
| Designed library R2 primer | CGG TCT CGG CAT TCC TGC TGA ACC GCT CTT CCG ATC T (SEQ |
| | ID NO:35) |
| Predicted target library R2 primer | CGA CGG CGT ACA CTT CTA TTC TGT CTT CCC GCG TCC G (SEQ |
| | ID NO: 36) |
| antisense stall sequence index | GGA TCC AGG AAC GTC TTC CAT ACA ACC TCC TTA CTA CAT |
| primer (i5) | (SEQ ID NO: 37) |
| designed library index primer (17) | AGA TCG GAA GAG CGG TTC AGC AGG AAT GCC GAG ACC G |
| | (SEQ ID NO: 38) |
| predicted target library index | CGG ACG CGG GAA GAC AGA ATA GAA GTG TAC GCC GTC G |
| primer (i7) | (SEQ ID NO: 39) |
| | Sequence |
| miR-21 Knockout sgRNAs | RNA in italic; AlTR1, IDT Alt-R guide RNA modification |
| upstream miR-21 sgRNA | /AlTR1/UGA UAA GCU ACC CGA CAA GGG UUU UAG AGC UAU GCU |
| | /AlTR2/ (SEQ ID NO: 40) |
| downstream miR-21 sgRNA | /AlTR1/CGA UGG GCU GUC UGA CAU UUG UUU UAG AGC UAU GCU |
| | /AlTR2/ (SEQ ID NO: 41) |
| miR-21 plasmid library cloning | Sequence |
| primers | DNA |
| CMV forward primer with EcoRI | AAA TTC GAA TTC GCA AAA TTT AAG CTA CAA CAA GGC AAG G |
| site | (SEQ ID NO: 42) |
| CMV reverse primer with eGFP | TGG TGG CAT AGG TAC CTA ACT GAT AGG GAG AGC TCT GCT TA |
| homology | (SEQ ID NO: 43) |
| eGFP forward primer with CMV | GGT ACT GTT GGT AAA GCC ACC TTA GGT ACC TAT GCC ACC |
| homology | ATG GTG (SEQ ID NO: 44) |
| eGFP reverse primer with SV40 | GGG ATC CCG ACT TGC TAG CCA CTT AGG TAG TAA TCC GCA |
| homology | TGC TCT AGA TTA CTT GTA CAG (SEQ ID NO: 45) |
| SV40 forward primer with eGFP | TAC TAC CTA AGT GGC TAG CAA GTC GGG ATC CCT GGC TGC |
| homology | TGC CAC CGC TGA GCA ATA ACT (SEQ ID NO: 46) |
| SV40 reverse primer with XhoI | TAA ACC TCG AGT ACC GCA CAG ATG CGT AAG GAG AAA ATA |
| site | CCG (SEQ ID NO: 47) |
| PB vector backbone forward | ACC AAC GAA TTC GGA AGG ATC TGC GAT CGC TCC GGT GC (SEQ |
| primer with EcoRI site | ID NO: 48) |
| PB vector backbone reverse | TAA ATA CTC GAG TGG CTG TCC CTC ATA AAA GTT TTG (SEQ ID |
| primer with XhoI site | NO: 49) |
| Reporter library sequencing | Sequence |
| primers | DNA |
| Reporter library R1 seqiencing | TAA ATA GCT AGC TAA GAA GAC GTT CCT GGT TCC (SEQ ID |
| primer | NO: 50) |
| Reporter library R2 sequencing | AAA CAA GGA TCC AAA TGA ACC GCT CTT CCG ATC (SEQ ID |
| primer | NO: 51) |
| Reporter library index 1 | GAT CGG AAG AGC GGT TCA TTT GGA TCC TTG TTT (SEQ ID |
| sequencing primer (i7) | NO: 52) |
| Reporter library index 2 | GGA ACC AGG AAC GTC TTC TTA GCT AGC TAT TTA (SEQ ID |
| sequencing primer (i5) | NO: 53) |

Processing sequencing data. Following sequencing, tile and x, y coordinates of each cluster were extracted. Clusters were deemed library members based on aligning a segment of the read 2 sequence (either 5′-AGA TCG GAA GAG CGG TTC AG-3′ (SEQ ID NO:1) or 5′-CGG ACG CGG GAA GAC AGA AT-3′ (SEQ ID NO:2)). Fiducial marks were identified by aligning the exact fiducial sequence (5′-TAG CCA GCC TGA TAA GTA ACA CCA CCA CTG-3′ (SEQ ID NO:3)). Fiducial marks and library members identified in this manner were used for registering tiles prior to experiments and for registering sequencing data to images during image processing. Because all library members were shorter than the read sequence, each variant was fully sequenced twice during sequencing. Only clusters that exactly matched a known library sequence in both reads were fit in downstream data analysis for determination of kₒₙ and K_{D}.

RISC purification. S100 extract was generated from SV40 large T-antigen immortalized AGO2^{-/-} MEFs that stably overexpress mouse AGO2 (O'Carroll et al., 2007). Cell extract was essentially prepared as described (Dignam et al. 1983). Briefly, the cell pellet was washed three times in ice-cold PBS and once in Buffer A (10 mM HEPES-KOH (pH 7.9), 10 mM potassium acetate, 1.5 mM magnesium acetate, 0.01% w/v CHAPS, 0.5 mM DTT, 1 mM AEBSF, hydrochloride, 0.3 μM Aprotinin, 40 μM Bestatin, hydrochloride, 10 μM E-64, 10 μM Leupeptin hemisulfate). The supernatant was removed, and 0.11 cell pellet volumes of Buffer B (300 mM HEPES-KOH (pH 7.9), 1.4 M potassium acetate, 30 mM magnesium acetate, 0.01% w/v CHAPS, 0.5 mM DTT, 1 mM AEBSF, hydrochloride, 0.3 μM Aprotinin, 40 μM Bestatin, hydrochloride, 10 μM E-64, 10 μM Leupeptin, hemisulfate) was added, followed by centrifugation at 100,000×g for 20 min at 4° C. Ice-cold 80% (w/v) glycerol was then added to achieve a 20% (w/v) final glycerol concentration, followed by gentle inversion to mix. S100 was aliquoted, frozen in liquid nitrogen, and stored at −80° C.

To load AGO2-RISC, 30 pM duplex siRNA with a 3′ Alexa Fluor 555 (Life Technologies) labeled guide strand was incubated in S100 extract for 1.5 h at 37° C. in 15 mM HEPES-KOH (pH 7.9), 100 mM potassium acetate, 5 mM magnesium acetate, 5 mM DTT, 1 mM ATP, 25 mM creatine phosphate, 30 μg ml⁻¹ creatine kinase. RISC was purified as described (Flores-Jasso et al., 2013). Briefly, the assembled AGO2-RISC was incubated overnight at 4° C. with a biotinylated, 2′-O-methyl capture oligonucleotide linked to streptavidin paramagnetic beads (Dynabeads MyOne Streptavidin T1, Life Technologies). RISC was eluted with a competitor oligonucleotide for 2 h at room temperature. Excess competitor oligonucleotide was removed by incubating the eluate with streptavidin paramagnetic beads (Dynabeads MyOne Streptavidin T1, Life Technologies) for 15 min at room temperature. The RISC was concentrated, and the potassium acetate concentration was adjusted to 100 mM (f.c.) by centrifugal ultrafiltration (Amicon Ultra-centrifugal filter, 10K MWCO, EMD Millipore, Billerica, Mass.). The concentration of active, purified RISC was measured by pre-steady-state target cleavage assays at 23° C. in the presence of 100 pM ³²P-radiolabeled target RNA. The concentration of catalytically inactive, purified RISC was measured by fluorescence with Typhoon FLA-7000 (GE Healthcare) following denaturing polyacrylamide gel electrophoresis.

Imaging station setup. A custom instrument that enables biochemical measurements to be made in a MiSeq flow cell was constructed as described in (She et al., 2017). The camera, lasers, Z-stage, XY-stage, syringe pump, and objective lens used in the instrument were salvaged from an Illumina GAIIx. These parts were combined with a fluidics adaptor designed to interface with Illumina MiSeq chips, a temperature control system, and laser control electronics to enable real time biochemical measurements in MiSeq flow cells. Imaging was performed using either a 400 ms exposure time at 150 mW fiber input power of a 660 nm laser and a 664 nm long pass filter (Semrock) or with a 600 ms exposure time at 150 mW input power of a 530 nm laser and a 590 nm center wave length and 104 nm guaranteed minimum 93% bandwidth band pass filter (Semrock).

Generation of RNA on the sequencing flow cell. MiSeq flow cells containing sequenced libraries were loaded into the custom imaging station for in situ RNA generation (Buenrostro et al., 2014, She et al., 2017). All steps were executed using custom xml scripts to control the imaging station's pump, stage movement, Peltier heater, lasers, and camera. Unless otherwise stated, all wash volumes were 100 μl and flowed at 100 μl min⁻¹.

Regeneration of double-stranded DNA. For the first experiment after sequencing, DNA not covalently attached to the flow cell surface was removed by heating the flow cell to 55° C. and washing with 100% (v/v) formamide. The flow cell was then heated to 60° C. and incubated in Cleavage buffer (80 mM Tris-HCl pH 8.0, 80 mM NaCl, 0.05% v/v Tween 20, 100 mM TCEP) for 10 min to remove residual fluorescence from sequencing reversible terminators.

Cy3-labeled fiducial mark oligonucleotides and 5′ biotinylated oligonucleotides (Table 1) were hybridized to the distal end of library ssDNA molecules in multiple phases. First, the flow cell was incubated in Hybridization buffer (5×SSC buffer (ThermoFisher 15557036), 5 mM EDTA, 0.05% v/v Tween 20) containing 500 pM of each oligonucleotide for 12 min at 60° C., followed by 12 min at 40° C. The flow cell was washed in Annealing buffer (1×SSC, 5 mM EDTA, 0.05% v/v Tween 20), and then incubated in Annealing buffer containing 500 pM of each oligonucleotide for 8 min at 40° C. Following oligonucleotide hybridization, the temperature was lowered to 37° C. and the flow cell was washed with Klenow buffer (1×NEB buffer 2 (NEB B7002S), 250 mM of each dNTP, 0.01% v/v Tween 20). The hybridized oligonucleotides were extended into dsDNA by adding one line volume (65 μl) of Klenow buffer containing 0.2 U/μl Klenow fragment (3′→5′ exo-minus (NEB M0212)) and pumping 9 μl of Klenow buffer every 5 min for a total of 30 min. Following dsDNA generation, the flow cell was washed with Hybridization buffer.

Because the success of RNA generation was determined by annealing of a labeled stall oligonucleotide to the nascent RNA molecule, it was necessary to block this DNA sequence in the event that dsDNA generation was less than 100% efficient. Blocking of the ssDNA stall sequence was achieved by incubating the flow cell in Hybridization buffer containing 500 pM unlabeled stall oligonucleotide for 10 min, washing with annealing buffer, and then incubating the flow cell in Annealing buffer containing 500 pM unlabeled stall oligonucleotide for 10 min. After another Annealing buffer wash, the flow cell was incubated in Annealing buffer containing 500 pM of labeled stall oligonucleotide for 10 min. The flow cell was imaged after this step to serve as a baseline image for RNA generation.

RNA generation. After dsDNA generation, the flow cell was incubated for 5 min in 1 μM streptavidin (PROzyme, SA10) in Annealing buffer. The streptavidin binds the biotinylated oligonucleotides used for dsDNA generation and stalls E. coli RNA polymerase holoenzyme (RNAP; NEB M0551) during RNA generation. After washing with Annealing buffer, the flow cell was incubated for 5 min in 5 μM biotin (ThermoFisher B20656) in Annealing buffer to saturate the remaining streptavidin binding sites. The flow cell was washed again with Annealing buffer, and then washed with Initiation buffer (2.5 μM each of ATP, GTP, and UTP in R-reaction buffer (20 mM Tris-HCl pH 7.5, 7 mM MgCl₂, 20 mM NaCl, 0.1 mM EDTA, 1.5% glycerol, 0.01% v/v Tween 20, 0.5 mM DTT)). One line volume (65 μl) of Initiation buffer containing 0.06 U/μl of RNAP was applied to the flow cell, after which 9 μl of Initiation buffer was pumped every 100 sec for a total of 10 min. Because the Initiation buffer lacks CTP, RNAP is allowed to initiate transcription on dsDNA molecules containing the T7A1 sequence, but then stalls part way through transcribing the stall sequence. Unbound RNAP was then removed from the flow cell with an Initiation buffer wash. RNAP was extended by adding Extension buffer (10 mM NTPs in R-reaction buffer) containing 500 pM each of labeled stall DNA oligonucleotide and R2 DNA blocking oligonucleotides (Table 1) and incubating for 5 min. The labeled stall oligonucleotide binds to the 5′ end of the newly transcribed RNA molecule and serves the dual purpose of blocking this common sequence while also allowing for assessment of RNA generation efficiency. The R2 oligonucleotides serve to block the 3′ common sequence of each RNA molecule, leaving only the variable target sequences single stranded. To ensure efficient blocking, the flow cell is incubated in 500 pM of each oligonucleotide in Blocking buffer (1×SSC, 7 mM MgCl₂, 0.05% v/v Tween 20) for an additional 10 min. Finally, the flow cell was washed with Ago Sample buffer (30 mM HEPES-KOH (pH 7.3), 120 mM potassium acetate, 3.5 mM magnesium acetate, 1 mM DTT, 50 μg/mL BSA, 10 μg/mL yeast tRNAs, 0.05% v/v Tween 20).

Measurement of association rates and equilibrium dissociation constants on chip. After RNA was transcribed in the MiSeq flow cell, AGO2 loaded with a labeled guide was introduced at various concentrations to measure association kinetics. For let-7a, association was measured at 63 pM, 125 pM, 250 pM, and 500 pM for the entire library. For miR-21, association was measured at 25 pM, 188 pM, 375 pM, and 1 pM for the second part of the library, and at 50 pM, 125 pM, 250 pM, and 500 pM for the initial library.

Tiles were imaged continuously during the first 20 min of association, with each tile being imaged approximately every 90 sec. For association experiments lasting longer than 20 min, additional images were taken at log spaced intervals. By collecting association data at multiple concentrations, we were able to fit association constants and were able to use the fraction bound at the end of each association to construct equilibrium binding curves.

After each association experiment, the chip was washed with 500 μl Wash buffer (10 mM Tris-HCl pH 8.0, 5 mM EDTA, 0.05% v/v Tween 20), and then all protein, RNA and non-covalently attached DNA was stripped by heating the chip to 55° C. and flowing 100% formamide. RNA was regenerated for each subsequent experiment.

Measurement of cleavage rates—Transcription of library. To construct the target libraries for the cleavage experiments, a T7 promoter was added by PCR to the DNA oligonucleotide-pool library designed for the array experiment. RNA target libraries were transcribed with T7 RNA Polymerase for 3 h using the following conditions: 16 mM MgCl₂, 2 mM Spermidine, 40 mM Tris-HCl pH 7.5, 0.01% Triton X-100, 2 mM each dNTP, and 40 mM DTT. The resulting products were treated with DNase-I and purified using Qiagen RNeasy Mini columns. For let-7a the full designed library and the library of predicted targets in the short sequence context was used for cleavage experiments. However, for miR-21, only the initial designed library (˜7,000 variants), containing the less degenerate sequences for which cleavage is more relevant, was used for the cleavage experiments.

Measurement of cleavage rates—cleavage experimental protocol. Cleavage assays were performed in cleavage buffer (30 mM HEPES-KOH (pH 7.3), 120 mM potassium acetate, 3.5 mM MgCl₂, 1 mM DTT, and 0.1% v/v Tween-20). Prior to the reactions, DNA blocking oligonucleotides were annealed to the target RNA primer sequences to prevent structure formation or interaction between the primer sequence and the protein by adding 1.25×excess blocking oligonucleotides to the RNA in cleavage buffer without MgCl₂. The resulting mixture was heated to 70° C. and cooled slowly to room temperature (10 min) to anneal the oligonucleotides to the RNA. After annealing the oligonucleotides, the RNA target libraries were diluted to the reaction concentrations and MgCl₂ concentration was adjusted to 3.5 mM. For each reaction, the RNA target library concentration was set to 10% of the protein concentration to ensure that there would be minimal depletion of protein. The miR-21 reactions were performed at 8 pM RISC and the let-7a reactions were performed at 4 pM RISC. High concentrations of RISC were used to limit the effects of association on the observed cleavage rate such that for the vast majority of target variants the rate measured would reflect the single turnover cleavage rate constant. Reactions were initiated by mixing the protein and target libraries at 37° C. and incubating for log spaced amounts of time ranging from 15 sec to 32 min. Additionally, one reaction was immediately quenched after mixing the components and a no protein control went through the same procedure. The reactions were quenched at —80° C. and once all reactions were complete, they were immediately placed at 95° C. to denature the protein and prevent any additional cleavage in the downstream library generation steps. The reactions were then treated with DNase-I to remove the blocking oligonucleotides and the resulting RNA was reverse transcribed with superscript IV reverse transcriptase. The resulting cDNA was barcoded for each time point using NEBNext 2×high-fidelity master mix and 250 pM of each timepoint barcode. PCR progress was monitored by including 0.6×SYBR Green in the reaction and stopped when the SYBR Green signal began to plateau to minimize the total number of PCR cycles to prevent introduction of bias at this step. The resulting libraries were purified using Qiagen QL&quick PCR Purification columns and. quantified for sequencing with qPCR (see Assembly and Sequencing of Library Above)

Measurement of cleavage rates — sequencing of cleavage libraries. Paired end sequencing (2×36) of the resulting libraries was performed with 75 bp High Output Next Seq kits on a NextSeq500. Custom read 1, 2, and index primers were spiked into the run to sequence the cleavage libraries.

Cell culture. HEK-293 Flp-In T-REx cells (Invitrogen) were cultured in DMEM with 10% FBS, GlutaMAX, and penicillin-streptomycin. Cells were maintained in a humidified CO₂ incubator at 37° C. and examined regularly to ensure absence of mycoplasma contamination.

Generation of miR-21 knockout cell line. Cas9-gRNA ribonucleoprotein complexes containing two tracrRNA:crRNAs flanking the miR-21 hairpin (5′-TGA TAA GCT ACC CGA CAA GGT GG-3; 5′-CGA TGG GCT GTC TGA CAT TTT GG-3′) were transfected into HEK-293 Flp-In T-REx cells according to the Alt-R CRISPR-Cas9 user guide (IDT), except that RNAiMAX was replaced with Lipofectamine 3000 (Invitrogen). Transfected cells were incubated for 48 h, after which single cells were sorted into 96-well plates. After 3 weeks, viable clones were genotyped using primers that flanked the miR-21 hairpin (5′-TCA AAT CCT GCC TGA CTG TCT G-3′ and 6═-CCA GAG TTT CTG ATT ATA AAC AAT GAT GC-3′). Homozygous edited clones were further expanded and deletion of the miR-21 hairpin was confirmed by amplification and electrophoresis of the miR-21 locus and by a TaqMan RT-qPCR miRNA assay specific for mature miR-21 (Applied Biosystems).

Preparation of miR-21 library piggyBac reporter constructs. All oligonucleotides used to construct the miR-21 plasmid library are reported in Table 1. The CMV promoter, eGFP coding sequence, and SV40 poly(A) signal sequence were amplified from existing plasmids in the lab. Each of these components was amplified using primers containing homology arms to neighboring segments, and an EcoRI site was added upstream of the CMV promoter and a Xhol site was added downstream of the SV40 poly(A) sequence. The promoter, gene, and poly(A) signal sequence were assembled using NEBuilder HiFi DNA Assembly master mix with equimolar mixing of components. After assembly, the full gene was amplified further using only the outermost primers.

The PB-U6insert-EF1puro backbone was amplified such that the U6 promoter was removed and an EcoRI site was added upstream of the EF1 promoter and an XhoI site was added inside of the 5′ piggyBac right (3′) inverted repeat. The reporter gene was inserted into the amplified PB-EF1puro backbone to create the PB-CMV-GFP-EF1puro plasmid, wherein the CMV and EF1 promoters faced in opposite directions.

To prepare the miR-21 target sequences for cloning into the PB-CMV-GFP-EF1puro plasmid, the complete array library was used as a template. The variable target region of the library was amplified 15 cycles using primers that introduced restriction sites on each end of the target. The library was then cloned 61 bases downstream of the GFP stop codon and 93 bases upstream of the SV40 poly(A) signal sequence.

Stable transfection of miR-21 target library. miR-21 knockout cells were grown to 90% confluency in a 6-well tissue culture plate. 200 ng of purified miR-21 target plasmid library was co-transfected with or without 200 ng Super piggyBac Transposase Expression Vector (SBI) using lipofectamine 3000 (Invitrogen) according to manufacturer's instructions. After 24 h, transfected cells were passaged into a 10-cm tissue culture plates. After another 24 h, culture media was replaced with culture media containing 2 μg/mL Puromycin. Media was replaced every 3 days until the negative control cells (those without Transposase expression vector co-transfection) were all dead.

Knockdown in cells. miR-21 knockout cells containing the miR-21 target library were plated in six-well plates at 300,000 cells per well. After 24 h, cells were transfected with variable miR-21 siRNA (Dharmacon) concentrations (100, 20, 4, 0.8, 0.16, or 0.032 pM) using lipofectamine 3000 (Invitrogen) according to manufacturer's instructions. Cells were incubated for 48 h, after which RNA was isolated from each well using a Quick-RNA MiniPrep kit (Zymo). On-column DNase I treatment was performed for all samples according to manufacturer's recommendation. RNA was then reverse-transcribed using superscript IV reverse transcriptase and an RT primer specific to the region immediately 3′ to the variable region of the miR-21 target reporter constructs. The resulting cDNA was barcoded and prepared for sequencing as described for the in vitro cleavage libraries. Paired-end sequencing was performed as described above for in vitro cleavage libraries.

### Example 15 - Quantification and Statistical Analysis

Data processing and imaging fitting. To map sequencing data to array experimental images, the previously extracted tile and coordinate information was cross-correlated to images iteratively. This process resulted in cluster coordinates being mapped to images at sub-pixel resolution as previously described (Denny et al., 2018; She et al., 2017). After coordinate mapping, each cluster was fit to a two-dimensional Gaussian to quantify fluorescence.

Association curve fitting. Following quantification of cluster intensity at each time point, association rates were fit for each variant. As imaging all DNA clusters required 18 images to be taken, the time for each image was set as the median time for the 18 images taken in that round of imaging. To account for variability between illumination and focus in each imaging cycle, the fluorescence intensity at each timepoint was normalized by dividing by the median fluorescence intensity of a fiducial mark (a fluorescent DNA oligonucleotide hybridized directly to single stranded DNA) that otherwise should have constant fluorescence intensity during the experiment. Association rates were determined by fitting the following single exponential to the median fluorescence of all clusters representing a single molecular variant at each timepoint:

f_{intensity}=(f_{eq}−fₘᵢₙ)*(1−e^{-k}^{_{obs}}^{t})+fₘᵢₙ

where f_{intensity} is the fluorescence intensity, f_{eq} is the fluorescence intensity at infinite time, fₘᵢₙ is the fluorescence intensity at time 0, and k_{obs} is the observed rate. Least-squares fitting here and for the equilibrium and cleavage fitting below was carried out using the python package lmfit.

Error in the measurement of the observed rates was estimated by bootstrapping the clusters representing each molecular variant. All clusters representing a single variant were sampled with replacement and the median fluorescence of the resampled clusters was fit to the above equation. This was repeated 1,000 times to generate 95% confidence intervals on the observed rate constant fits.

After computing the observed association rates, the observed rates for each variant were fit to the following equation to compute the association rate:

k_{obs}=kₒₙ*[RISC]+k_{off}

where k_{obs} is the observed rate, kₒₙ is the association constant, k_{off} is the dissociation constant, and [RISC] is the concentration of loaded AGO2.

Equilibrium binding curve fitting—initial fitting of single clusters. The maximum fluorescence values determined by fitting the association experiments at each concentration were used to fit equilibrium dissociation constants. Using fit f_{eq} values, which represent the equilibrium binding at infinite time, ensured that the values used to fit equilibrium binding curves represented the amount of binding at equilibrium for each concentration. Since the K_{D} of the perfectly complementary target was well below any concentration that we performed an experiment at, we normalized the f_{eq} values for all variants to the f_{eq} value of the perfectly complementary sequence at a given concentration. This allowed us to account for differences between experiments related to RNA production, illumination, and fiducial mark signal. The equilibrium fluorescence values at each concentration were fit to the following equation to determine the dissociation constant:

$${f}_{intensity} = ( {f}_{max} - {f}_{min} ) * ( \frac{[ RISC ]}{[ RISC ] + {K}_{d}} ) + {f}_{min}$$

where f_{intensity} is the fluorescence intensity, fₘₐₓ is the fluorescence intensity when the target is fully bound, fₘᵢₙ is the fluorescence intensity of the unbound target, [RISC] is the concentration of loaded AGO2, and K_{D} is the dissociation constant. Since the fₘᵢₙ was very low for all variants it was constrained to be between 0 and 2 percent of the fully bound signal for the perfectly complementary target.

Equilibrium binding curve fitting—determination of fₘₐₓ distribution. After fitting this equation for all variants, it was necessary to account for uncertainty in the true fₘₐₓ value for variants that were not fully bound at the highest experimental concentration. To do this we estimated the distribution of fₘₐₓ values across all variants. This distribution was estimated by selecting all variants with K_{D}'s less than 30 pM, which should be fully saturated at the highest experimental concentration. The f_{eq} values for all of these variants at the highest concentration was then used as the fₘₐₓ distribution.

Equilibrium binding curve fitting—bootstrapping to estimate K_{D} and Error. To estimate the K_{D} and error for each molecular variant we first determined if the fₘₐₓ distribution needed to be enforced. In cases where the maximum fluorescence achieved at any concentration exceed the lower limit of the 95% confidence interval of the fₘₐₓ distribution, the fₘₐₓ value was allowed to float during fitting. For these variants, the f_{eq} values at each concentration were sampled from the f_{eq} values determined when bootstrapping the association rate fits. This was repeated 100 times to generate 95% confidence intervals for the equilibrium constant. The variants that did not reach a significant fluorescence level at any concentration were also fit by sampling the f_{eq} values at each concentration from the f_{eq} values determined when bootstrapping the association rate fits. However, rather than allowing the fₘₐₓ value to float, a value was selected from the fₘₐₓ distribution determined with the high-affinity targets (above), and the fₘₐₓ was constrained to this value during fitting. The final equilibrium constant was set to the median of these 100 fits and the 95% confidence interval was defined from all the fit values.

Cleavage rate fitting. Sequencing data was first converted to counts for sequences in the designed library that had the same sequence in both read1 and read2. A set of highly degenerate normalization sequences was used to normalize the counts for different sequencing depth at each time point using the following formula:

$${counts}_{i , normalized} = {counts}_{i} \frac{{median ( normalization counts )}_{0}}{{median ( normalization counts )}_{i}}$$

For miR-21, the normalization sequences included 10 sequences with long stretches of central and seed mismatches. For let-7a, since the library tested for cleavage was much larger, all sequences with nucleotides t7-t11 mismatched were used as normalization sequences. Following normalization, the counts for each variant were fit to the following single exponential equation to determine the cleavage rate:

counts_{i,normalized}=(countsₘₐₓ−countsₘᵢₙ)e^{-k}^{_{cleave}}^{t}+countsₘᵢₙ

where countsₘₐₓ is the counts at time 0, countsₘᵢₙ is the counts at infinite time, and k_{cleave} is the single turnover cleavage rate. Variants for which the mean of the final 3 time points was greater than the mean of the first two time points or the overall change in counts was less than 15% of the median number of counts were defined as non-cleavers (k_{cleave}<0.0002 s⁻¹) due to the insufficient loss of signal throughout the experiment. The cleavage data was fit to an alternative model that incorporated both binding and cleavage. The follow model:

$$\frac{d [ RISC : RNA ]}{dt} = {k}_{on} [ RNA ] [ RISC ] - {k}_{off} [ RISC : mRNA ] - {k}_{cleave} [ RISC : RNA ] \\ \frac{d [ RNA ]}{dt} = - {k}_{on} [ RNA ] [ RISC ] + {k}_{off} [ RISC : RNA ]$$

was fit to the counts data using the relative association rates measured in the RNA array experiments and the dissociation rates determined from the association rates and dissociation constants measured in the RNA array experiments.

RNA-Seq data analysis. The raw counts table that included RNA-Seq with (“AL7_Inp_rep1”, “AL7_Inp_rep2”) and without (“AC_Inp_rep1”,“AC_Inp_rep2”a) a let-7a decoy was downloaded from ArrayExpress (E-MTAB-5386). Log₂(control/let-7a decoy) was computed with DESeq2. Only genes containing an average of 10 counts or more in these 4 samples were used for downstream analysis.

Comparison of see type binding affinity. 3′ UTRs for the Gencode transcripts identified as representative transcripts in TargetScan were downloaded for mm10. We scanned through each 3′ UTR and counted the number of 6mer, 7mer-A1, 7mer-m8, and 8mer seed sequences. We selected transcripts containing only one canonical seed site and compared the median loge change for each of class of seed types to the median affinity measured for each canonical seed type.

Model fitting—alignment of sequences. A dynamic programming approach was used to align the target and guide sequences. The following parameters were defined for the dynamic programming: a parameter for each nucleotide at positions t1-t21 (84 total) that are referred to as Eₚₙ where p is the guide position that the target is bound to (t1-t21) and n is the nucleotide (A, C, G, or U); parameters for opening target and guide bulges in the seed, central, and 3′ supplemental region (6 total); parameters for extension of target and guide bulges in the seed, central, and 3′ supplemental region (6 total); and a parameter for initiation of pairing following a bulge or mismatch. The parameters used were inspired by findings in binding experiments.

Initialize four matrices (N_{target}×N_{guide}) to track the cases where the final subproblem ends with a match (M), a mismatch (N), a target bulge (T) , and a guide bulge (G). For all matrices, the rows (i) represent the position in the target and the columns (j) represent the position in the guide. Row 1 of the match matrix is then initialized as follows:

M_{1,j}=E_{j,t}_{₁}

Where t₁ is the target nucleotide at position 1. Column 1 of the match matrix was initialized as:

M_{j,1}=E_{1,t}_{ⱼ}

The following recursions were then used to populate the four matrices:
If tᵢ,gⱼ complementary:

N_{i,j}=∞

M_{i,j}=E_{j,t}_{ᵢ}+min(M_{i-1,j-1}, N_{i-1,j-1}+Pᵢₙᵢₜ, T_{i-1,j-1}+Pᵢₙᵢₜ, G_{i-1,j-1}+Pᵢₙᵢₜ, Pᵢₙᵢₜ)

otherwise:

M_{i,j}=∞

N_{i,j}=E_{j,t}_{ᵢ}+min(M_{i-1,j-1}, N_{i-1,j-1})

G_{i,j}=min(M_{i,j-1}+GB_{opening,j}, T_{i,j-1}+GB_{extension,j}, N_{i,j-1}+GB_{opening,j})

T_{i,j}=min(M_{i-1,j}+TB_{opening,j}, T_{i-1,j}+TB_{extension,j}, N_{i-1,j}+TB_{opening,j})

These recursions allowed us to identify the best register ending with a match at tᵢ:gⱼ, a mismatch at tᵢ:gⱼ, and a guide or target bulge at tᵢ:gⱼ. Following population of the four matrices, the minimum value in the matched matrix was selected as the most likely binding register. From the minimum entry in the matrix, a traceback to identify the steps taken to get to that point was performed, enabling to reconstruction of the optimal binding register.

Model fitting—model for AGO2 binding affinity. To fit a model predicting RISC binding model, all miR-21 and let-7a sequences were aligned with the above method. Features were defined for each base at each position (21 positions×4 bases/position=84 parameters), for opening target and guide bulges in the seed, central, and 3′ supplemental region (2 strands×3 regions=6 parameters), for extension of target and guide bulges in the seed, central, and 3′ supplemental region (2 strands×3 regions=6 parameters), and for initiation of pairing following a bulge or mismatch. One additional feature used to account for RNA secondary structure was also included. This feature was calculated as the difference in the energy of the ensemble of RNA secondary structures formed when the seed region was involved in structure and when the seed region was constrained to be unstructured. These RNA structure predictions were made using the following commands in RNAfold. For the case of no constraint (no region forced to not form structure): RNAfold-T 37 C-p0—noPS inputfile.fa. and for the case when a constraint was included: RNAfold-T 37 C-p0—noPS-C inputfile.fa. Each of these commands were provided with a fasta file containing the RNA targets and, for the case where constraints were included they were indicated as:

(SEQ ID NO: 4)
UUUUUACUAUACAACCUCCUACCUCAUUUUU

For fitting, data was filtered to only include RNA targets for which we could quantitatively measure a binding constant (10 pM>K_{D}>10 pM) and only sequences of length 39 nucleotides or less. Testing and training sets of equal size were randomly selected from the filtered data. All fitting was done using scikit-learn module in Python 2.7. The model was fit with Ridge regression to prevent parameters from being fit to large, non-physical values. All fits were performed with an intercept, which represents the intrinsic affinity of the protein for any nucleic acid strand.

Model fitting—fitting of cleavage model. For miR-21 RISC, cleavage data was only collected on the original library (7,675 unique sequences; see assembly and sequencing of library above) that included primarily single mismatches, double mismatches, triple mismatches, insertions of different lengths, single and double deletions, combinatorial insertions, and structured and context variants. We filtered out the structured and context variants when doing the model fitting since this would have introduced many occurrences of the perfect complement target. We filtered the let-7 cleavage data to include the same classes of variants as the miR-21 data. This enabled comparison of the two models performance, building and testing of a general model with similar data from both guides. Additionally, we are primarily interested in predicting cleavage for highly complementary sequences, and most of the remainder of the library probes questions relevant to miRNA binding but, since many of these targets have large numbers of mismatches, little cleavage activity is observed. Prior to fitting the data was filtered to remove targets that we did not measure a cleavage rate for (k_{cleave}<0.0001 s⁻¹) and targets that had a poor goodness of fit (r²<0.6).

To fit the cleavage model, we first aligned all miR-21 and let-7a target sequences. After alignment we defined features for each mismatch at each position and for guide and target bulges at each positions. We performed a constrained fit when fitting the models for let-7a and miR-21 specific cleavage. The mismatch penalties were constrained to be no more than 2 natural logs below and 1 natural log above the observed single mismatch penalties during fitting. The guide and target bulge penalties were constrained to be no more than 1.5 natural logs below and 0.5 natural log above the observed single bulge penalties during fitting. The model was then fit to the single mismatches, double mismatches, single position target insertions, and single deletions using the lmfit module in Python 2.7. Following fitting of models for let-7 and miR-21, a general model was fit to both datasets. This model included the same bulge parameters as the guide specific models, but only included position specific parameters for transitions and transversions since the base depends on the microRNA/siRNA. This model was fit to all single and double mismatched targets and single insertions and deletions of let-7a and miR-21 and tested on triple mismatched targets and targets with multiple insertions and deletions for both sequences. Fitting of this model was performed with ridge regression in scikit-learn in Python 2.7.

Analysis of siRNA efficacy in cells. Sequence data was converted to counts and normalized as described above for in vitro cleavage data. The change in the abundance of each target sequence i for each condition j was calculated to be:

$$fold {change}_{i , j} = \frac{normalized {counts}_{i , j}}{normalized {counts}_{i , 0}}$$

Where normalized counts_{i,0} is the normalized count of target i in the mock transfected cells.

Biochemical model derivation and fitting. We aimed to predict mRNA steady state knockdown with a kinetic model of RISC activity. This approach has the benefit of not requiring any assumptions about the concentration of target RNA relative to the Kₘ of the interaction (the free ligand approximation)—a significant limitation of many classical biochemical models of enzyme activity. For a given miR-21 target, we considered the following molecular species and rates:

[mRNA], concentration of unbound target mRNA

[RISC], concentration of unbound, miR-21-loaded RISC

[RISC:mRNA], concentration of loaded RISC bound to target mRNA

[RISC:cutRNA], concentration of loaded RISC bound to cut mRNA

kₜᵣₐₙₛ, mRNA transcription rate

k_{degrade}, basal mRNA degradation rate

kₒₙ, association rate of RISC for target mRNA

k_{off}, dissociation rate of RISC from target mRNA

k_{decay}, rate of miRNA accelerated mRNA decay, not through direct cleavage

k_{cleave}, single-turnover cleavage rate for RISC on target mRNA

kᵣₑₗₑₐₛₑ, rate of product release

We considered four rate equations describing RISC activity:

$$1 ) \frac{d [ mRNA ]}{dt} = {k}_{trans} - {k}_{degrade} [ mRNA ] - {k}_{on} [ mRNA ] [ RISC ] + {k}_{off} [ RISC : mRNA ] \\ 2 ) \frac{d [ RISC ]}{dt} = - {k}_{on} [ RISC ] [ mRNA ] + {k}_{off} [ RISC : mRNA ] + {k}_{decay} [ RISC : mRNA ] + {k}_{release} [ RISC : cutRNA ] \\ 3 ) \frac{d [ RISC : mRNA ]}{dt} = {k}_{on} [ mRNA ] [ RISC ] - {k}_{off} [ RISC : mRNA ] - {k}_{decay} [ RISC : mRNA ] - {k}_{cleave} [ RISC : mRNA ] \\ 4 ) \frac{d [ RISC : cutRNA ]}{dt} = {k}_{cleave} [ RISC : mRNA ] - {k}_{release} [ RISC : cutRNA ]$$

The quantity measured in the in cell knockdown assay is the total uncleaved RNA, or [RISC:mRNA]+[mRNA]. By setting each of the above equations to 0 and solving the system of equations, it can be shown that:

$$5 ) [ RISC : mRNA ] + [ mRNA ] = \frac{( \frac{{k}_{on} [ RISC ]}{{k}_{cleave} + {k}_{decay} + {k}_{off}} + 1 ) * ( \frac{{k}_{trans}}{{k}_{decay} + {k}_{cleave}} )}{( \frac{{k}_{degrade}}{{k}_{decay} + {k}_{cleave}} + \frac{{k}_{on} [ RISC ]}{{k}_{cleave} + {k}_{decay} + {k}_{off}} )}$$

The maximum possible mRNA concentration [mRNAmax] was assumed to occur in the absence of any miR-21 siRNA:

$$6 ) \frac{d [ mRNA ]}{dt} = 0 = {k}_{trans} - {k}_{degrade} [ mRNA ] \\ [ {mRNA}_{max} ] = \frac{{k}_{trans}}{{k}_{degrade}}$$

Therefore, the change in the abundance of a target mRNA is given by:

$$7 ) \frac{[ RISC : mRNA ] + [ mRNA ]}{[ {mRNA}_{max} ]} = \frac{( \frac{{k}_{on} [ RISC ]}{{k}_{cleave} + {k}_{decay} + {k}_{off}} + 1 ) * ( \frac{{k}_{degrade}}{{k}_{decay} + {k}_{cleave}} )}{( \frac{{k}_{degrade}}{{k}_{decay} + {k}_{cleave}} + \frac{{k}_{on} [ RISC ]}{{k}_{cleave} + {k}_{decay} + {k}_{off}} )}$$

This equation contains three unknown parameters: k_{decay}, k_{degrade}, and [RISC]. Because all targets were placed in a nearly identical gene context, we assumed that k_{decay} and k_{degrade} are constant across all targets and all miR-21 transfections. The free RISC concentration [RISC] was constrained to be at most the transfected miR-21 concentration, and was fit for each transfection condition. We observed that many targets had little knockdown in cells despite having in vitro cleavage rates >10-fold faster than their corresponding dissociation rates. We surmised that the in cell dissociation rates might be significantly faster than the measured in vitro rates. To account for this, we added a dissociation rate scaling term C, which was fit as a constant across all targets and all transfection conditions:

$$8 ) repression = \frac{1}{fold change} = \frac{( \frac{{k}_{degrade}}{{k}_{decay} + {k}_{cleave}} + \frac{{k}_{on} [ RISC ]}{{k}_{cleave} + {k}_{decay} + {k}_{off} * C} )}{( \frac{{k}_{on} [ RISC ]}{{k}_{cleave} + {k}_{decay} + {k}_{off} * C} + 1 ) * ( \frac{{k}_{degrade}}{{k}_{decay} + {k}_{cleave}} )}$$

This model was fit using experimentally measured relative association rates and cleavage rates for each target. Dissociation rates were inferred from model predicted affinities. To limit differential effects of structure or other RNA binding proteins on the targets examined, only targets containing five adenosines flanking the targets region and that had values for all of the required parameters were used in model fitting and subsequent analyses (4,483 sequences).

### REFERENCES

Agarwal, V., Bell, G. W., Nam, J.-W., and Bartel, D. P. (2015). Predicting effective microRNA target sites in mammalian mRNAs. eLife 4.
Ameres, S. L., Martinez, J., and Schroeder, R. (2007). Molecular basis for target RNA recognition and cleavage by human RISC. Cell 130, 101-112.
Baek, D., Villén, J., Shin, C., Camargo, F. D., Gygi, S. P., and Bartel, D. P. (2008). The impact of microRNAs on protein output. Nature 455, 64-71.
Bartel, D. P. (2009). MicroRNAs: target recognition and regulatory functions. Cell 136, 215-233.
Bartel, D. P. (2018). Metazoan MicroRNAs. Cell 173, 20-51.
Bazzini, A. A., Lee, M. T., and Giraldez, A. J. (2012). Ribosome profiling shows that miR-430 reduces translation before causing mRNA decay in zebrafish. Science 336, 233-237.
Betel, D., Koppal, A., Agius, P., Sander, C., and Leslie, C. (2010). Comprehensive modeling of microRNA targets predicts functional non-conserved and non-canonical sites. Genome Biol. 11, R90.

### Bisaria, N., Jarmoskaite, I., and Herschlag, D. (2016). Specificity Principles in RNA-Guided Targeting.

Buenrostro, J. D., Araya, C. L., Chircus, L. M., Layton, C. J., Chang, H. Y., Snyder, M. P., and Greenleaf, W. J. (2014). Quantitative analysis of RNA-protein interactions on a massively parallel array reveals biophysical and evolutionary landscapes. Nat. Biotechnol. 32, 562-568.
Chakraborty, C., Sharma, A. R., Sharma, G., Doss, C. G. P., and Lee, S.-S. (2017). Therapeutic miRNA and siRNA: Moving from Bench to Clinic as Next Generation Medicine. Mol. Ther. Nucleic Acids 8, 132-143.
Chi, S. W., Zang, J. B., Mele, A., and Darnell, R. B. (2009). Argonaute HITS-CLIP decodes microRNA-mRNA interaction maps. Nature 460, 479-486.
Chi, S. W., Hannon, G. J., and Darnell, R. B. (2012). An alternative mode of microRNA target recognition. Nat. Struct. Mol. Biol. 19, 321-327.
Clark, P. M., Loher, P., Quann, K., Brody, J., Londin, E. R., and Rigoutsos, I. (2014). Argonaute CLIP-Seq reveals miRNA targetome diversity across tissue types. Sci. Rep. 4, 5947.
Deerberg, A., Willkomm, S., and Restle, T. (2013). Minimal mechanistic model of siRNA-dependent target RNA slicing by recombinant human Argonaute 2 protein. Proc. Natl. Acad. Sci. U. S. A. 110, 17850-17855.
Denny, S. K., Bisaria, N., Yesselman, J. D., Das, R., Herschlag, D., and Greenleaf, W. J. (2018). High-Throughput Investigation of Diverse Junction Elements in RNA Tertiary Folding. Cell 174, 377-390.e20.
Doench, J. G. (2003). siRNAs can function as miRNAs. Genes Dev. 17, 438-442.
Doench, J. G., and Sharp, P. A. (2004). Specificity of microRNA target selection in translational repression. Genes Dev. 18, 504-511.
Dowdy, S. F. (2017). Overcoming cellular barriers for RNA therapeutics. Nat. Biotechnol. 35, 222-229.
Dykxhoorn, D. M., Palliser, D., and Lieberman, J. (2006). The silent treatment: siRNAs as small molecule drugs. Gene Ther. 13, 541-552.
Elbashir, S. M., Harborth, J., Lendeckel, W., Yalcin, A., Weber, K., and Tuschl, T. (2001). Duplexes of 21-nucleotide RNAs mediate RNA interference in cultured mammalian cells. Nature 411, 494-498.
Elkayam, E., Kuhn, C.-D., Tocilj, A., Haase, A. D., Greene, E. M., Hannon, G. J., and Joshua-Tor, L. (2012). The structure of human argonaute-2 in complex with miR-20a. Cell 150, 100-110.
Frank, F., Sonenberg, N., and Nagar, B. (2010). Structural basis for 5′-nucleotide base-specific recognition of guide RNA by human AGO2. Nature 465, 818-822.
Friedman, R. C., -H. Farh, K. K., Burge, C. B., and Bartel, D. P. (2008). Most mammalian mRNAs are conserved targets of microRNAs. Genome Res. 19, 92-105.
Grimson, A., Farh, K. K.-H., Johnston, W. K., Garrett-Engele, P., Lim, L. P., and Bartel, D. P. (2007). MicroRNA targeting specificity in mammals: determinants beyond seed pairing. Mol. Cell 27, 91-105.
Grosswendt, S., Filipchyk, A., Manzano, M., Klironomos, F., Schilling, M., Herzog, M., Gottwein, E., and Raewsky, N. (2014). Unambiguous identification of miRNA:target site interactions by different types of ligation reactions. Mol. Cell 54, 1042-1054.
Guo, H., Ingolia, N. T., Weissman, J. S., and Bartel, D. P. (2010). Mammalian microRNAs predominantly act to decrease target mRNA levels. Nature 466, 835-840.
Haley, B., and Zamore, P. D. (2004). Kinetic analysis of the RNAi enzyme complex. Nat. Struct. Mol. Biol. 11, 599-606.
Hammond, S. M., Bernstein, E., Beach, D., and Hannon, G. J. (2000). An RNA-directed nuclease mediates post-transcriptional gene silencing in Drosophila cells. Nature 404, 293-296.
Helwak, A., Kudla, G., Dudnakova, T., and Tollervey, D. (2013). Mapping the human miRNA interactome by CLASH reveals frequent noncanonical binding. Cell 153, 654-665.
Hendrickson, D. G., Hogan, D. J., McCullough, H. L., Myers, J. W., Herschlag, D., Ferrell, J. E., and Brown, P. O. (2009). Concordant regulation of translation and mRNA abundance for hundreds of targets of a human microRNA. PLoS Biol. 7, e1000238.
Hutvágner, G., and Zamore, P. D. (2002). A microRNA in a multiple-turnover RNAi enzyme complex. Science 297, 2056-2060.
Jo, M. H., Shin, S., Jung, S.-R., Kim, E., Song, J.-J., and Hohng, S. (2015). Human Argonaute 2 Has Diverse Reaction Pathways on Target RNAs. Mol. Cell 59, 117-124.
Khorshid, M., Hausser, J., Zavolan, M., and van Nimwegen, E. (2013). A biophysical miRNA-mRNA interaction model infers canonical and noncanonical targets. Nat. Methods 10, 253-255.
Krek, A., Grun, D., Poy, M. N., Wolf, R., Rosenberg, L., Epstein, E. J., MacMenamin, P., da Piedade, I., Gunsalus, K. C., Stoffel, M., et al. (2005). Combinatorial microRNA target predictions. Nat. Genet. 37, 495-500.

Lewis, B. P., Shih, I.-H., Jones-Rhoades, M. W., Bartel, D. P., and Burge, C. B. (2003). Prediction of mammalian microRNA targets. Cell 115, 787-798.

Loeb, G. B., Khan, A. A., Canner, D., Hiatt, J. B., Shendure, J., Darnell, R. B., Leslie, C. S., and Rudensky, A. Y. (2012). Transcriptome-wide miR-155 binding map reveals widespread noncanonical microRNA targeting. Mol. Cell 48, 760-770.

Lorenz, R., Bernhart, S. H., Siederdissen, C. H. zu, Tafer, H., Flamm, C., Stadler, P. F., and Hofacker, I. L. (2011). ViennaRNA Package 2.0. Algorithms Mol. Biol. 6, 26.

Luna, J. M., Scheel, T. K. H., Danino, T., Shaw, K. S., Mele, A., Fak, J. J., Nishiuchi, E., Takacs, C. N., Catanese, M. T., de Jong, Y. P., et al. (2015). Hepatitis C virus RNA functionally sequesters miR-122. Cell 160, 1099-1110.

Ma, J.-B., Yuan, Y.-R., Meister, G., Pei, Y., Tuschl, T., and Patel, D. J. (2005). Structural basis for 5′-end-specific recognition of guide RNA by the A. fulgidus Piwi protein. Nature 434, 666-670.

Mayr, C., and Bartel, D. P. (2009). Widespread shortening of 3′ UTRs by alternative cleavage and polyadenylation activates oncogenes in cancer cells. Cell 138, 673-684.

Parker, J. S., Mark Roe, S., and Barford, D. (2005). Structural insights into mRNA recognition from a PIWI domain—siRNA guide complex. Nature 434, 663-666.

Pfister, E. L., Kennington, L., Straubhaar, J., Wagh, S., Liu, W., DiFiglia, M., Landwehrmeyer, B., Vonsattel, J.-P., Zamore, P. D., and Aronin, N. (2009). Five siRNAs targeting three SNPs may provide therapy for three-quarters of Huntington's disease patients. Curr. Biol. 19, 774-778.

Reczko, M., Maragkakis, M., Alexiou, P., Grosse, I., and Hatzigeorgiou, A. G. (2012). Functional microRNA targets in protein coding sequences. Bioinformatics 28, 771-776.

Rivas, F. V., Tolia, N. H., Song, J.-J., Aragon, J. P., Liu, J., Hannon, G. J., and Joshua-Tor, L. (2005). Purified Argonaute2 and an siRNA form recombinant human RISC. Nat. Struct. Mol. Biol. 12, 340-349.

Salomon, W. E., Jolly, S. M., Moore, M. J., Zamore, P. D., and Serebrov, V. (2015). Single-Molecule Imaging Reveals that Argonaute Reshapes the Binding Properties of Its Nucleic Acid Guides. Cell 162, 84-95.

Schirle, N. T., and MacRae, I. J. (2012). The crystal structure of human Argonaute2. Science 336, 1037-1040.

Schirle, N. T., Sheu-Gruttadauria, J., and MacRae, I. J. (2014). Structural basis for microRNA targeting. Science 346, 608-613.

Schwarz, D. S., Ding, H., Kennington, L., Moore, J. T., Schelter, J., Burchard, J., Linsley, P. S., Aronin, N., Xu, Z., and Zamore, P. D. (2006). Designing siRNA that distinguish between genes that differ by a single nucleotide. PLoS Genet. 2, e140.
Selbach, M., Schwanhäusser, B., Thierfelder, N., Fang, Z., Khanin, R., and Rajewsky, N. (2008). Widespread changes in protein synthesis induced by microRNAs. Nature 455, 58-63.
She, R., Chakravarty, A. K., Layton, C. J., Chircus, L. M., Andreasson, J. O. L., Damaraju, N., McMahon, P. L., Buenrostro, J. D., Jarosz, D. F., and Greenleaf, W. J. (2017). Comprehensive and quantitative mapping of RNA-protein interactions across a transcribed eukaryotic genome. Proceedings of the National Academy of Sciences 114, 3619-3624.
Sheng, G., Gogakos, T., Wang, J., Zhao, H., Serganov, A., Juranek, S., Tuschl, T., Patel, D. J., and Wang, Y. (2017). Structure/cleavage-based insights into helical perturbations at bulge sites within T. thermophilus Argonaute silencing complexes. Nucleic Acids Res. 45, 9149-9163.
Shin, C., Nam, J.-W., Farh, K. K.-H., Chiang, H. R., Shkumatava, A., and Bartel, D. P. (2010). Expanding the microRNA targeting code: functional sites with centered pairing. Mol. Cell 38, 789-802.
Tang, G., Reinhart, B. J., Bartel, D. P., and Zamore, P. D. (2003). A biochemical framework for RNA silencing in plants. Genes Dev. 17, 49-63.
Tomari, Y., and Zamore, P. D. (2005). Perspective: machines for RNAi. Genes Dev. 19, 517-529.
Wang, W., Yoshikawa, M., Han, B. W., Izumi, N., Tomari, Y., Weng, Z., and Zamore, P. D. (2014). The Initial Uridine of Primary piRNAs Does Not Create the Tenth Adenine that Is the Hallmark of Secondary piRNAs. Mol. Cell 56, 708-716.
Wang, Y., Juranek, S., Li, H., Sheng, G., Tuschl, T., and Patel, D. J. (2008). Structure of an argonaute silencing complex with a seed-containing guide DNA and target RNA duplex. Nature 456, 921-926.
Wang, Y., Juranek, S., Li, H., Sheng, G., Wardle, G. S., Tuschl, T., and Patel, D. J. (2009). Nucleation, propagation and cleavage of target RNAs in Ago silencing complexes. Nature 461, 754-761.
Wee, L. M., Flores-Jasso, C. F., Salomon, W. E., and Zamore, P. D. (2012). Argonaute divides its RNA guide into domains with distinct functions and RNA-binding properties. Cell 151, 1055-1067.
Werfel, S., Leierseder, S., Ruprecht, B., Kuster, B., and Engelhardt, S. (2017). Preferential microRNA targeting revealed by in vivo competitive binding and differential Argonaute immunoprecipitation. Nucleic Acids Res. 45, 10218-10228.

Wittrup, A., and Lieberman, J. (2015). Knocking down disease: a progress report on siRNA therapeutics. Nat. Rev. Genet. 16, 543-552.

Zamore, P. D., Tuschl, T., Sharp, P. A., and Bartel, D. P. (2000). RNAi: double-stranded RNA directs the ATP-dependent cleavage of mRNA at 21 to 23 nucleotide intervals. Cell 101, 25-33.

Zeng, Y., Wagner, E. J., and Cullen, B. R. (2002). Both natural and designed micro RNAs can inhibit the expression of cognate mRNAs when expressed in human cells. Mol. Cell 9, 1327-1333.

One or more features from any embodiments described herein or in the figures may be combined with one or more features of any other embodiment described herein in the figures without departing from the scope of the disclosure.

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

1. An inhibitory RNA polynucleotide comprising between 15 and 30 nucleotides,
wherein the inhibitory RNA polynucleotide is partially complementary to an equal length portion of a target gene,
wherein the inhibitory RNA polynucleotide comprises at least one mismatched nucleotide at position 5, 7, 8, 12, 16, 17, 18, 19, 20, or 21, and
wherein the inhibitory RNA polynucleotide guides an RNA-induced silencing complex (RISC) to cleave the target gene.

2. The inhibitory RNA polynucleotide of claim 1, where the inhibitory RNA polynucleotide comprises at least one mismatched nucleotide at position 12, 16, 17, 18, 19, 20, or 21.

3. The inhibitory RNA polynucleotide of claim 1, where the inhibitory RNA polynucleotide comprises one mismatched nucleotide at position 12 and/or at position 18.

4. (canceled)

5. The inhibitory RNA polynucleotide of claim 1, wherein the inhibitory RNA polynucleotide comprises at least two mismatched nucleotides at positions selected from 5, 7, 8, 12, 15, 16, 17, 18, 19, 20, and 21.

6. The inhibitory RNA polynucleotide of claim 5, wherein the inhibitory RNA polynucleotide comprises two mismatched nucleotides, and wherein:
(a) a first mismatched nucleotide is at position 12 and a second mismatched nucleotide is at position 5, 7, 8, 15, 16, 17, 18, 19, 20, or 21; or
(b) a first mismatched nucleotide is at position 18 and a second mismatched nucleotide is at position 5, 7, 8, 12, 15, 16, 17, 19, 20, or 21; or
(c) a first mismatched nucleotide is at position 12 and a second mismatched nucleotide is at position 18.

7-8. (canceled)

9. The inhibitory RNA polynucleotide of claim 1, wherein the inhibitory RNA polynucleotide comprises at least two mismatched nucleotides at positions selected from positions 15, 16, 17, 18, 19, 20, and 21.

10. The inhibitory RNA polynucleotide of claim 9, wherein:
(a) the inhibitory RNA polynucleotide comprises mismatched nucleotides at positions 15, 16, 17, 18, 19, 20, and 21; or
(b) the inhibitory RNA polynucleotide comprises mismatched nucleotides at positions 17, 18, 19, 20, and 21.

11. (canceled)

12. The inhibitory RNA polynucleotide of claim 1, wherein the inhibitory RNA polynucleotide guides the RISC to cleave the target gene at a faster cleavage rate than the corresponding cleavage rate of RISC when RISC is guided by a corresponding inhibitory RNA polynucleotide having complete complementarity to the equal length portion of the target gene.

13. The inhibitory RNA polynucleotide of claim 1, wherein the inhibitory RNA polynucleotide is single-stranded or double-stranded.

14. (canceled)

15. A pharmaceutical composition comprising an inhibitory RNA polynucleotide of claim 1 and a pharmaceutically acceptable carrier.

16. The pharmaceutical composition of claim 15, wherein the inhibitory RNA polynucleotide is encapsulated in a nanoparticle.

17. The pharmaceutical composition of claim 16, wherein the nanoparticle is a liposome.

18. The pharmaceutical composition of claim 17, wherein the liposome is a polyethylene glycol (PEG) liposome.

19. A method of increasing the cleavage rate of an RNA-induced silencing complex (RISC) in cleaving a target gene, comprising introducing at least one mismatched nucleotide to an inhibitory RNA polynucleotide comprising between 15 and 30 nucleotides,
wherein the inhibitory RNA polynucleotide is partially complementary to an equal length portion of a target gene,
wherein the inhibitory RNA polynucleotide comprises at least one mismatched nucleotide at position 5, 7, 8, 12, 16, 17, 18, 19, 20, or 21, and
wherein the RISC is guided by the inhibitory RNA polynucleotide to bind and cleave the target gene at a faster cleavage rate than the corresponding cleavage rate of RISC when RISC is guided by a corresponding inhibitory RNA polynucleotide having complete complementarity to the equal length portion of the target gene.

20. The method of claim 19, wherein the inhibitory RNA polynucleotide comprises at least two mismatched nucleotides at positions selected from positions 5, 7, 8, 12, 15, 16, 17, 18, 19, 20, and 21.

21. A method of decreasing the cleavage rate of an RNA-induced silencing complex (RISC) in cleaving a target gene, comprising introducing at least two mismatched nucleotides to an inhibitory RNA polynucleotide comprising between 15 and 30 nucleotides,
wherein the inhibitory RNA polynucleotide is partially complementary to an equal length portion of a target gene,
wherein the inhibitory RNA polynucleotide comprises at least two mismatched nucleotides at positions selected from positions 9, 10, 11, and 13, and
wherein the RISC is guided by the inhibitory RNA polynucleotide to bind and cleave the target gene at a slower cleavage rate than the corresponding cleavage rate of RISC when RISC is guided by a corresponding inhibitory RNA polynucleotide having complete complementarity to the equal length portion of the target gene.

22. A method of decreasing the expression level of a target gene in a cell, comprising contacting the cell with an inhibitory RNA polynucleotide of claim 1.

23. A method of treating a disease in a subject in need thereof, comprising administered to the subject an inhibitory RNA polynucleotide of claim 1,
wherein the inhibitory RNA polynucleotide decreases the expression level of the target gene.

24. The method of claim 23, wherein the inhibitory RNA polynucleotide comprises at least two mismatched nucleotides at positions selected from positions 5, 7, 8, 9, 10, 12, 13, 15, 16, 17, 18, 19, 20, and 21.

25. A method of synthesizing an inhibitory RNA polynucleotide of claim 1, comprising:
(a) providing a sequence of the target gene;
(b) selecting a portion of the sequence of the target gene where the inhibitory RNA polynucleotide binds;
(c) selecting at least one position from positions 5, 7, 8, 12, 16, 17, 18, 19, 20, and 21 of the inhibitory RNA polynucleotide to introduce a mismatched nucleotide at the position; and
(c) introducing the mismatched nucleotide at the selected position of the inhibitory RNA polynucleotide during synthesis of the inhibitory RNA polynucleotide,
wherein the inhibitory RNA polynucleotide is partially complementary to an equal length portion of the target gene, and
wherein an RNA-induced silencing complex (RISC) is guided by the inhibitory RNA polynucleotide to bind and cleave the target gene at a faster cleavage rate than the corresponding cleavage rate of RISC when RISC is guided by a corresponding inhibitory RNA polynucleotide having complete complementarity to the equal length portion of the target gene.


SEQUENCE LISTING (80 sequences)

SEQ ID NO: 1 - DNA, 20 bp, Artificial Sequence
  Description of Artificial Sequence Synthetic oligonucleotide
agatcggaagagcggttcag

SEQ ID NO: 2 - DNA, 20 bp, Artificial Sequence
  Description of Artificial Sequence Synthetic oligonucleotide
cggacgcgggaagacagaat

SEQ ID NO: 3 - DNA, 30 bp, Artificial Sequence
  Description of Artificial Sequence Synthetic oligonucleotide
tagccagcctgataagtaacaccaccactg

SEQ ID NO: 4 - RNA, 31 nt, Artificial Sequence
  Description of Artificial Sequence Synthetic oligonucleotide
uuuuuacuauacaaccuccuaccucauuuuu

SEQ ID NO: 5 - RNA, 21 nt, Artificial Sequence
  Description of Artificial Sequence Synthetic oligonucleotide
uauacaaccuacuaccugcuu

SEQ ID NO: 6 - RNA, 21 nt, Artificial Sequence
  Description of Artificial Sequence Synthetic oligonucleotide
ugagguaguagguuguauagu

SEQ ID NO: 7 - RNA, 22 nt, Artificial Sequence
  Description of Artificial Sequence Synthetic oligonucleotide
ucaacaucagucugauaagcuu

SEQ ID NO: 8 - RNA, 22 nt, Artificial Sequence
  Description of Artificial Sequence Synthetic oligonucleotide
uagcuuaucagacugauguuga

SEQ ID NO: 9 - RNA, 31 nt, Artificial Sequence
  Description of Artificial Sequence Synthetic oligonucleotide
auagacugcgacaauagccuaccuccgaacg

SEQ ID NO: 10 - DNA, 31 bp, Artificial Sequence
  Description of Artificial Sequence Synthetic oligonucleotide
cgttcggaggtaggctattgtcgcagtctat

SEQ ID NO: 11 - RNA, 31 nt, Artificial Sequence
  Description of Artificial Sequence Synthetic oligonucleotide
gaugaaccacucagagacauaagcuaaucua

SEQ ID NO: 12 - DNA, 31 bp, Artificial Sequence
  Description of Artificial Sequence Synthetic oligonucleotide
tagattagcttatgtctctgagtggttcatc

SEQ ID NO: 13 - DNA, 47 bp, Artificial Sequence
  Description of Artificial Sequence Synthetic primer
gcgtaatacgactcactataggggtcctttgatcgtgacaaaacaat

SEQ ID NO: 14 - DNA, 106 bp, Artificial Sequence
  Description of Artificial Sequence Synthetic primer
cccatttaggtgacactatagatttatacctagttaaacagcggaactgtgtataaaaggttgaggtagtaggttgtatagtatccagaggaattcattatcagtg

SEQ ID NO: 15 - DNA, 107 bp, Artificial Sequence
  Description of Artificial Sequence Synthetic primer
cccatttaggtgacactatagatttacatctagttgaggtgcggaactgtgtataaaaggttagcttatcagactgatgttgaatccagaggaattcattatcagtg

SEQ ID NO: 16 - DNA, 121 bp, Artificial Sequence
  Description of Artificial Sequence Synthetic polynucleotide
  modified_base (53)..(68): a, c, t, g, unknown or other
aatgatacggcgaccaccgagatctacacactggtatgcgagacgcaggatgnnnnnnnnnnnnnnnnatttatcaaaaagagtattgacttaaagtctaacctataggatacttacagcc

SEQ ID NO: 17 - DNA, 92 bp, Artificial Sequence
  Description of Artificial Sequence Synthetic oligonucleotide
atttatcaaaaagagtattgacttaaagtctaacctataggatacttacagccatgtagtaaggaggttgtatggaagacgttcctggatcc

SEQ ID NO: 18 - DNA, 61 bp, Artificial Sequence
  Description of Artificial Sequence Synthetic oligonucleotide
caagcagaagacggcatacgagatcggtctcggcattcctgctgaaccgctcttccgatct

SEQ ID NO: 19 - DNA, 61 bp, Artificial Sequence
  Description of Artificial Sequence Synthetic oligonucleotide
caagcagaagacggcatacgagatcgacggcgtacacttctattctgtcttcccgcgtccg

SEQ ID NO: 20 - DNA, 29 bp, Artificial Sequence
  Description of Artificial Sequence Synthetic oligonucleotide
aatgatacggcgaccaccgagatctacac

SEQ ID NO: 21 - DNA, 24 bp, Artificial Sequence
  Description of Artificial Sequence Synthetic oligonucleotide
caagcagaagacggcatacgagat

SEQ ID NO: 22 - DNA, 39 bp, Artificial Sequence
  Description of Artificial Sequence Synthetic primer
atgtagtaaggaggttgtatggaagacgttcctggatcc

SEQ ID NO: 23 - DNA, 37 bp, Artificial Sequence
  Description of Artificial Sequence Synthetic primer
cggtctcggcattcctgctgaaccgctcttccgatct

SEQ ID NO: 24 - DNA, 37 bp, Artificial Sequence
  Description of Artificial Sequence Synthetic primer
cgacggcgtacacttctattctgtcttcccgcgtccg

SEQ ID NO: 25 - DNA, 61 bp, Artificial Sequence; identical to SEQ ID NO: 18
  Description of Artificial Sequence Synthetic oligonucleotide

SEQ ID NO: 26 - DNA, 61 bp, Artificial Sequence; identical to SEQ ID NO: 19
  Description of Artificial Sequence Synthetic oligonucleotide

SEQ ID NO: 27 - DNA, 39 bp, Artificial Sequence
  Description of Artificial Sequence Synthetic oligonucleotide
ggatccaggaacgtcttccatacaacctccttactacat

SEQ ID NO: 28 - DNA, 39 bp, Artificial Sequence; identical to SEQ ID NO: 27
  Description of Artificial Sequence Synthetic oligonucleotide

SEQ ID NO: 29 - DNA, 32 bp, Artificial Sequence
  Description of Artificial Sequence Synthetic oligonucleotide
tcggcattcctgctgaaccgctcttccgatct

SEQ ID NO: 30 - DNA, 32 bp, Artificial Sequence
  Description of Artificial Sequence Synthetic oligonucleotide
cgacggcgtacacttctattctgtcttcccgc

SEQ ID NO: 31 - DNA, 57 bp, Artificial Sequence
  Description of Artificial Sequence Synthetic oligonucleotide
taatacgactcactatagatgtagtaaggaggttgtatggaagacgttcctggatcc

SEQ ID NO: 32 - DNA, 37 bp, Artificial Sequence; identical to SEQ ID NO: 23
  Description of Artificial Sequence Synthetic primer

SEQ ID NO: 33 - DNA, 37 bp, Artificial Sequence; identical to SEQ ID NO: 24
  Description of Artificial Sequence Synthetic primer

SEQ ID NO: 34 - DNA, 39 bp, Artificial Sequence; identical to SEQ ID NO: 22
  Description of Artificial Sequence Synthetic primer

SEQ ID NO: 35 - DNA, 37 bp, Artificial Sequence; identical to SEQ ID NO: 23
  Description of Artificial Sequence Synthetic primer

SEQ ID NO: 36 - DNA, 37 bp, Artificial Sequence; identical to SEQ ID NO: 24
  Description of Artificial Sequence Synthetic primer

SEQ ID NO: 37 - DNA, 39 bp, Artificial Sequence; identical to SEQ ID NO: 27
  Description of Artificial Sequence Synthetic primer

SEQ ID NO: 38 - DNA, 37 bp, Artificial Sequence
  Description of Artificial Sequence Synthetic primer
agatcggaagagcggttcagcaggaatgccgagaccg

SEQ ID NO: 39 - DNA, 37 bp, Artificial Sequence
  Description of Artificial Sequence Synthetic primer
cggacgcgggaagacagaatagaagtgtacgccgtcg

SEQ ID NO: 40 - RNA, 36 nt, Artificial Sequence
  Description of Artificial Sequence Synthetic oligonucleotide
ugauaagcuacccgacaaggguuuuagagcuaugcu

SEQ ID NO: 41 - RNA, 36 nt, Artificial Sequence
  Description of Artificial Sequence Synthetic oligonucleotide
cgaugggcugucugacauuuguuuuagagcuaugcu

SEQ ID NO: 42 - DNA, 40 bp, Artificial Sequence
  Description of Artificial Sequence Synthetic primer
aaattcgaattcgcaaaatttaagctacaacaaggcaagg

SEQ ID NO: 43 - DNA, 41 bp, Artificial Sequence
  Description of Artificial Sequence Synthetic primer
tggtggcataggtacctaactgatagggagagctctgctta

SEQ ID NO: 44 - DNA, 45 bp, Artificial Sequence
  Description of Artificial Sequence Synthetic primer
ggtactgttggtaaagccaccttaggtacctatgccaccatggtg

SEQ ID NO: 45 - DNA, 60 bp, Artificial Sequence
  Description of Artificial Sequence Synthetic primer
gggatcccgacttgctagccacttaggtagtaatccgcatgctctagattacttgtacag

SEQ ID NO: 46 - DNA, 60 bp, Artificial Sequence
  Description of Artificial Sequence Synthetic primer
tactacctaagtggctagcaagtcgggatccctggctgctgccaccgctgagcaataact

SEQ ID NO: 47 - DNA, 42 bp, Artificial Sequence
  Description of Artificial Sequence Synthetic primer
taaacctcgagtaccgcacagatgcgtaaggagaaaataccg

SEQ ID NO: 48 - DNA, 38 bp, Artificial Sequence
  Description of Artificial Sequence Synthetic primer
accaacgaattcggaaggatctgcgatcgctccggtgc

SEQ ID NO: 49 - DNA, 36 bp, Artificial Sequence
  Description of Artificial Sequence Synthetic primer
taaatactcgagtggctgtccctcataaaagttttg

SEQ ID NO: 50 - DNA, 33 bp, Artificial Sequence
  Description of Artificial Sequence Synthetic primer
taaatagctagctaagaagacgttcctggttcc

SEQ ID NO: 51 - DNA, 33 bp, Artificial Sequence
  Description of Artificial Sequence Synthetic primer
aaacaaggatccaaatgaaccgctcttccgatc

SEQ ID NO: 52 - DNA, 33 bp, Artificial Sequence
  Description of Artificial Sequence Synthetic primer
gatcggaagagcggttcatttggatccttgttt

SEQ ID NO: 53 - DNA, 33 bp, Artificial Sequence
  Description of Artificial Sequence Synthetic primer
ggaaccaggaacgtcttcttagctagctattta

SEQ ID NO: 54 - DNA, 23 bp, Artificial Sequence
  Description of Artificial Sequence Synthetic oligonucleotide
tgataagctacccgacaaggtgg

SEQ ID NO: 55 - DNA, 23 bp, Artificial Sequence
  Description of Artificial Sequence Synthetic oligonucleotide
cgatgggctgtctgacattttgg

SEQ ID NO: 56 - DNA, 22 bp, Artificial Sequence
  Description of Artificial Sequence Synthetic primer
tcaaatcctgcctgactgtctg

SEQ ID NO: 57 - DNA, 29 bp, Artificial Sequence
  Description of Artificial Sequence Synthetic primer
ccagagtttctgattataaacaatgatgc

SEQ ID NO: 58 - RNA, 21 nt, Artificial Sequence
  Description of Artificial Sequence Synthetic oligonucleotide
aucgaauagucugacuacaac

SEQ ID NO: 59 - RNA, 63 nt, Artificial Sequence
  Description of Artificial Sequence Synthetic oligonucleotide
cguacgaguacucgucguacgcguacuacgaguacgacucguaguacgcguagucgucguagu

SEQ ID NO: 60 - RNA, 21 nt, Artificial Sequence
  Description of Artificial Sequence Synthetic oligonucleotide
acuccaucauccaacauauca

SEQ ID NO: 61 - RNA, 63 nt, Artificial Sequence
  Description of Artificial Sequence Synthetic oligonucleotide
cguaguacgaguagucguacgagucguacgaguagucgucguagucguacgcguacgagucgu

SEQ ID NO: 62 - RNA, 21 nt, Artificial Sequence
  Description of Artificial Sequence Synthetic oligonucleotide
agagcaucauccaacauauca

SEQ ID NO: 63 - RNA, 21 nt, Artificial Sequence
  Description of Artificial Sequence Synthetic oligonucleotide
acucguaguuccaacauauca

SEQ ID NO: 64 - RNA, 12 nt, Artificial Sequence
  Description of Artificial Sequence Synthetic oligonucleotide
  modified_base (12)..(12): a, c, u, g, unknown or other
aaaaaauguugn

SEQ ID NO: 65 - RNA, 27 nt, Artificial Sequence
  Description of Artificial Sequence Synthetic oligonucleotide
  modified_base (1)..(1): a, c, u, g, unknown or other
ncaacaucagucguauaagcuaaaaaa

SEQ ID NO: 66 - RNA, 32 nt, Artificial Sequence
  Description of Artificial Sequence Synthetic oligonucleotide
aaaaacaacauucagucugauaagcugaaaaa

SEQ ID NO: 67 - RNA, 32 nt, Artificial Sequence
  Description of Artificial Sequence Synthetic oligonucleotide
aaaaacaacaucuaggcugauaagcuaaaaaa

SEQ ID NO: 68 - RNA, 31 nt, Artificial Sequence
  Description of Artificial Sequence Synthetic oligonucleotide
cucuauacaacacaccuacuaucucuugaug

SEQ ID NO: 69 - RNA, 31 nt, Artificial Sequence
  Description of Artificial Sequence Synthetic oligonucleotide
auuuuuauacagaaccuacugccucaaacug

SEQ ID NO: 70 - RNA, 21 nt, Artificial Sequence
  Description of Artificial Sequence Synthetic oligonucleotide
acuauacaaccuacuaccuca

SEQ ID NO: 71 - RNA, 21 nt, Artificial Sequence
  Description of Artificial Sequence Synthetic oligonucleotide
caacaucagucugauaagcua

SEQ ID NO: 72 - RNA, 22 nt, Artificial Sequence
  Description of Artificial Sequence Synthetic oligonucleotide
uagcuuauucagacugauguug

SEQ ID NO: 73 - RNA, 23 nt, Artificial Sequence
  Description of Artificial Sequence Synthetic oligonucleotide
caacaccucagucugauaggcua

SEQ ID NO: 74 - RNA, 31 nt, Artificial Sequence
  Description of Artificial Sequence Synthetic oligonucleotide
cgacgaagguauacaaagagcccuaccucaa

SEQ ID NO: 75 - RNA, 20 nt, Artificial Sequence
  Description of Artificial Sequence Synthetic oligonucleotide
aucgaauagucugacuacaa

SEQ ID NO: 76 - RNA, 60 nt, Artificial Sequence
  Description of Artificial Sequence Synthetic oligonucleotide
cguacgaguacucgucguacgcguacuacgaguacgacucguaguacgcguagucgucgu

SEQ ID NO: 77 - RNA, 20 nt, Artificial Sequence
  Description of Artificial Sequence Synthetic oligonucleotide
ucgaauagucugacuacaac

SEQ ID NO: 78 - RNA, 60 nt, Artificial Sequence
  Description of Artificial Sequence Synthetic oligonucleotide
acgaguacucgucguacgcguacuacgaguacgacucguaguacgcguagucgucguagu

SEQ ID NO: 79 - RNA, 27 nt, Artificial Sequence
  Description of Artificial Sequence Synthetic oligonucleotide
  modified_base (27)..(27): a, c, u, g, unknown or other
aaaaacaacaucagucguauaagcuan

SEQ ID NO: 80 - RNA, 13 nt, Artificial Sequence
  Description of Artificial Sequence Synthetic oligonucleotide
  modified_base (1)..(1): a, c, u, g, unknown or other
nuagcuuaaaaaa